(19) United States
(12) Patent Application Publication
(10) Pub. No.: US 2023/0148096 A1
(43) Pub. Date: May 11, 2023

(54) YEAST-BASED VACCINES

(71) Applicant: The Trustees of Columbia University In The City of New York, New York, NY (US)

(72) Inventors: Virginia Cornish, New York, NY (US); Jiaming Qi, New York, NY (US)

(73) Assignee: The Trustees of Columbia University In The City of New York, New York, NY (US)

(21) Appl. No.: 18/090,083

(22) Filed: Dec. 28, 2022

Related U.S. Application Data
(63) Continuation of application No. PCT/US21/39671, filed on Jun. 29, 2021.
(60) Provisional application No. 63/045,633, filed on Jun. 29, 2020.

(51) Int. Cl. A61K 36/06; A61K 9/00; A61P 31/14; C07K 14/005; A61K 39/39

(52) U.S. Cl. CPC A61K 36/06; A61K 9/0043; A61P 31/14; C07K 14/005; A61K 39/39; A61K 2039/523

(57) ABSTRACT

The disclosed subject matter provides for vaccines against coronaviruses comprising one or more fungal cells genetically engineered to secrete an antigen derived from a coronavirus, e.g., SARS-CoV-2, in situ, and methods of use thereof.

### CROSS-REFERENCE TO RELATED APPLICATIONS

This application is a continuation of International Patent Application No. PCT/US2021/039671, filed on Jun. 29, 2021, which claims priority to U.S. Provisional Application No. 63/045,633, filed on Jun. 29, 2020, the contents of which are incorporated by reference in their entirety, and to each of which priority is claimed.

### SEQUENCE LISTING

The specification further incorporates by reference the Sequence Listing submitted herewith via EFS on Dec. 28, 2022. Pursuant to 37 C.F.R. § 1.52(e)(5), the Sequence Listing file, identified as 070050_6677.xml, is 65,050 bytes and was created on Dec. 28, 2022. The Sequence Listing, electronically filed herewith, does not extend beyond the scope of the specification and thus does not contain new matter.

### TECHNICAL FIELD

The present disclosure relates to vaccines that include a genetically-engineered yeast expressing an antigen of a coronavirus, e.g., SARS-CoV-2, and methods of use thereof.

### BACKGROUND

COVID-19 is caused by severe acute respiratory syndrome coronavirus 2 (SARS-CoV-2). SARS-CoV-2 was first discovered in late 2019 and has since infected more than 180 million individuals and caused more than 3.91 million deaths globally, with about 603,000 of those deaths occurring in the United States alone. Therefore, there is a need for a prophylactic or therapeutic vaccine against SARS-CoV-2 that has good immunogenicity, a long shelf-life, is safe and cost-effective and can be manufactured at a large scale.

### SUMMARY

The present disclosure provides genetically-engineered cells, e.g., fungal cells, that autonomously generate and/or secrete an antigen derived from a coronavirus for preventing and/or reducing the severity of an infection by a coronavirus in a subject. The present disclosure further provides methods for using such genetically-engineered fungal cells.

In one aspect, the present disclosure provides fungal cells genetically engineered to produce an antigen comprising a protein or fragment thereof derived from a coronavirus in situ, wherein the antigen is secreted from the fungal cell. In certain embodiments, the coronavirus is SARS-CoV-2, MERS-CoV or SARS-CoV. In certain embodiments, the coronavirus is SARS-CoV-2. In certain embodiments, the coronavirus is a variant of SARS-CoV-2. In certain embodiments, the variant of SARS-CoV-2 is selected from the group consisting of an alpha variant, a beta variant, a gamma variant, a delta variant, an epsilon variant and a combination thereof. For example, but not by way of limitation, the variant of SARS-CoV-2 is selected from the group consisting of B.1.1.7, B.1.351, P.1, B.1.427, B.1.429, B.1.617.2 and a combination thereof.

In certain embodiments, the antigen is secreted from the fungal cell by a secretory pathway of the fungal cell. In certain embodiments, the antigen is secreted from the fungal cell by the alpha-mating factor secretion pathway of the fungal cell.

In certain embodiments, the antigen comprises a spike (S) protein or fragment thereof derived from a coronavirus. In certain embodiments, the S protein or fragment thereof is the receptor-binding domain (RBD) of the S protein. In certain embodiments, the RBD comprises one or more amino acid substitutions as shown in Table 3. In certain embodiments, the RBD comprises one or more amino acid substitution to modify the glycosylation of the RBD and/or the disulfide bonds of the RBD. In certain embodiments, the antigen comprises an amino acid sequence that is about 80%, about 85%, about 90% or about 95% homologous to an amino acid sequence set forth in SEQ ID NOs: 12-22. In certain embodiments, the antigen comprises an amino acid sequence set forth in SEQ ID NOs: 12-22 or conservative substitutions thereof. In certain embodiments, the antigen is encoded by a nucleotide sequence that is about 70%, about 75%, about 80%, about 85%, about 90% or about 95% homologous to a nucleotide sequence set forth in SEQ ID NOs: 1-11. In certain embodiments, the antigen is encoded by a nucleotide sequence set forth in SEQ ID NOs: 1-11.

In certain embodiments, the genetically-engineered cell further expresses and/or secretes a cell targeting molecule. In certain embodiments, the cell targeting molecule is displayed on the surface of the genetically-engineered cell. Alternatively or additionally, the antigen further comprises a cell targeting molecule. In certain embodiments, the cell targeting molecule is a molecule for targeting the genetically-engineered fungal cell and/or antigen to an immune cell. In certain embodiments, the immune cell is located in a mucosal membrane and/or located in the Peyer's patches in the small intestine. In certain embodiments, the cell targeting molecule is selected from the group consisting of a C-terminal Clostridium perfringens Enterotoxin (C-CPE), a Cholera Toxin Subunit B (CTB), a heat-labile enterotoxin B subunit (LTB), a C-terminal Clostridium difficile toxin A (TxA(C314)), CRM197 (a diphtheria toxin mutant), fragment C of tetanus toxoid, a cholesteryl group-bearing pullulan (CHP), PT-9K/129G (a detoxified derivative of pertussis toxin) or mutants thereof and a combination thereof. In certain embodiments, the cell targeting molecule comprises a CTB.

In certain embodiments, the antigen is a fusion protein that comprises a cell targeting molecule coupled to the protein or fragment thereof derived from a coronavirus by a linker. In certain embodiments, the cell targeting molecule is coupled to the protein or fragment thereof derived from a coronavirus by a N-terminal linker, a C-terminal linker or a combination thereof. In certain embodiments, the linker comprises Gly (G) and Ser (S) amino acids, e.g., a (G4S)3 linker.

In certain embodiments, the genetically-engineered cell further expresses and/or secretes an adjuvant. In certain embodiments, the adjuvant is displayed on the surface of the genetically-engineered cell.

In certain embodiments, the genetically-engineered fungal cell is a species from a genus selected from the group consisting of Cladosporium, Aureobasidium, Aspergillus, Saccharomyces, Malassezia, Epicoccum, Candida, Penicillium, Wallemia, Pichia, Phoma, Cryptococcus, Fusarium, Clavispora, Cyberlindnera, Kluyveromyces, Hansenula, Yarrowia, Neurospora, Schizosaccharomyces, Blastobotrys, Zygosaccharomyces, Debaryomyces, Torulaspora, Hanseniaspora, Rhodotorula, Wickerhamomyces and Williopsis and a combination thereof. In certain embodiments, the fungal cell is a species selected from the group consisting of Saccharomyces cerevisiae, Saccharomyces boulardii, Saccharomyces mikatae, Saccharomyces kudriavzevii, Saccharomyces uvarum, Kluyveromyces lactis, Kluyveromyces marxianus, Kluyveromyces dobzhansky, Hansenula polymorpha, Yarrowia lipolytica, Neurospora crassa, Schizosaccharomyces pombe, Blastobotrys (Arxula) adeninivorans, Candida boldmu, Candida boidinii, Pichia methanolica, Pichia stipites, Zygosaccharomyces rouxii, Zygosaccharomyces bailii and Schwanniomyces (Debaryomyces) occidentalis. In certain embodiments, the fungal cell is Saccharomyces cerevisiae, Saccharomyces boulardii and/or Kluyveromyces lactis.

In another aspect, the present disclosure further provides methods for preventing and/or reducing the severity of an infection with a coronavirus in a subject. In certain embodiments, the coronavirus is SARS-CoV-2, MERS-CoV or SARS-CoV. In certain embodiments, the coronavirus is SARS-CoV-2. In certain embodiments, the coronavirus is a variant of SARS-CoV-2. In certain embodiments, the variant of SARS-CoV-2 is selected from the group consisting of an alpha variant, a beta variant, a gamma variant, a delta variant, an epsilon variant and a combination thereof. For example, but not by way of limitation, the variant of SARS-CoV-2 is selected from the group consisting of B.1.1.7, B.1.351, P.1, B.1.427, B.1.429, B.1.617.2 and a combination thereof. In certain embodiments, the method includes administering to the subject a genetically-engineered cell secreting an antigen disclosed herein. In certain embodiments, the genetically-engineered fungal cell administered by a method disclosed herein is formulated for parenteral administration, intraocular administration, intraaural administration, intranasal administration, oral administration, enteral administration, rectal administration, vaginal administration or topical administration. In certain embodiments, the genetically-engineered fungal cell is formulated for enteral administration. In certain embodiments, the genetically-engineered fungal cell is formulated for intranasal administration. In certain embodiments, the genetically-engineered fungal cell is administered once every two weeks. In certain embodiments, the method further includes administration of an adjuvant.

The present disclosure further provides pharmaceutical compositions that include one or more genetically-engineered fungal cells disclosed herein and a pharmaceutically acceptable carrier. In certain embodiments, the pharmaceutical composition is formulated for parenteral administration, intraocular administration, intraaural administration, intranasal administration, oral administration, enteral administration, rectal administration, vaginal administration or topical administration. In certain embodiments, the pharmaceutical composition is formulated for enteral administration. In certain embodiments, the pharmaceutical composition is formulated for intranasal administration. In certain embodiments, the pharmaceutical composition further comprises a cell targeting molecule. In certain embodiments, the pharmaceutical composition further comprises an adjuvant.

The present disclosure provides a kit comprising one or more genetically-engineered fungal cells disclosed herein, or one or more pharmaceutical compositions disclosed herein.

The present disclosure further provides uses of a genetically-engineered cell for preventing and/or reducing the severity of a coronavirus infection. In certain embodiments, the coronavirus is SARS-CoV-2, MERS-CoV or SARS-CoV. In certain embodiments, the coronavirus is SARS-CoV-2. In certain embodiments, the coronavirus is a variant of SARS-CoV-2. In certain embodiments, the variant of SARS-CoV-2 is selected from the group consisting of an alpha variant, a beta variant, a gamma variant, a delta variant, an epsilon variant and a combination thereof. For example, but not by way of limitation, the variant of SARS-CoV-2 is selected from the group consisting of B.1.1.7, B.1.351, P.1, B.1.427, B.1.429, B.1.617.2 and a combination thereof.

### BRIEF DESCRIPTION OF THE DRAWINGS

FIG. 1 shows an exemplary cloning, transformation and cell culturing workflow for Saccharomyces cerevisiae (S. cerevisiae) and Saccharomyces boulardii (S. boulardii).

FIG. 2 shows an exemplary cloning, transformation and cell culturing workflow for Kluyveromyces lactis (K. lactis).

FIG. 3 shows the SDS-PAGE and Western Blot of the RBD antigen secreted from transformed K. lactis.

FIG. 4 shows the Western Blot of the mutant RBD antigen secreted from transformed S. cerevisiae.

FIG. 5 shows the Western Blot of the mutant RBD antigen secreted from transformed S. boulardii.

FIG. 6 shows a schematic of an exemplary antigen of the present disclosure.

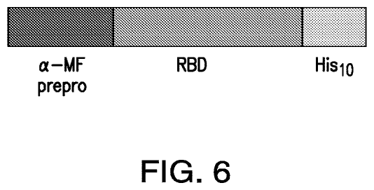

### DETAILED DESCRIPTION

The present disclosure provides genetically-engineered cells, e.g., fungal cells, where the genetically-engineered cells autonomously generate and/or secrete an antigen derived from a coronavirus for preventing and/or minimizing and/or reducing the severity of an infection by the coronavirus in a subject. The present disclosure further provides pharmaceutical compositions and kits of such genetically-engineered fungal cells.

For clarity, but not by way of limitation, the detailed description of the presently disclosed subject matter is divided into the following subsections:

I. Definitions;

II. Antigen;

III. Genetically-Engineered Cells;

IV. Methods of Use;

V. Pharmaceutical Compositions;

VI. Kits; and

VII. Exemplary Embodiments.

### I. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the present disclosure and how to make and use them.

As used herein, the use of the word “a” or “an” when used in conjunction with the term “comprising” in the claims and/or the specification can mean “one,” but it is also consistent with the meaning of “one or more,” “at least one,” and “one or more than one.”

The terms “comprise(s),” “include(s),” “having,” “has,” “can,” “contain(s),” and variants thereof, as used herein, are intended to be open-ended transitional phrases, terms or words that do not preclude additional acts or structures. The present disclosure also contemplates other embodiments “comprising,” “consisting of” and “consisting essentially of,” the embodiments or elements presented herein, whether explicitly set forth or not.

The term “about” or “approximately” means within an acceptable error range for the particular value as determined by one of ordinary skill in the art, which depends in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The terms “expression” or “expresses,” as used herein, refer to transcription and translation occurring within a cell, e.g., yeast cell. The level of expression of a gene and/or nucleic acid in a cell can be determined on the basis of either the amount of corresponding mRNA that is present in the cell or the amount of the protein encoded by the gene and/or nucleic acid that is produced by the cell. For example, mRNA transcribed from a gene and/or nucleic acid is desirably quantitated by northern hybridization. Sambrook et al., Molecular Cloning: A Laboratory Manual, pp. 7.3-7.57 (Cold Spring Harbor Laboratory Press, 1989). Protein encoded by a gene and/or nucleic acid can be quantitated either by assaying for the biological activity of the protein or by employing assays that are independent of such activity, such as western blotting or radioimmunoassay using antibodies that are capable of reacting with the protein. Sambrook et al., Molecular Cloning: A Laboratory Manual, pp. 18.1-18.88 (Cold Spring Harbor Laboratory Press, 1989).

As used herein, “polypeptide” refers generally to peptides and proteins having about three or more amino acids. In certain embodiments, the polypeptide can be endogenous to the cell, or preferably, can be exogenous, meaning that they are heterologous, i.e., foreign, to the cell being utilized, such as a synthetic peptide produced by a yeast cell. In certain embodiments, synthetic peptides are used, more preferably those which are directly secreted into the medium.

The term “protein” as used herein refers to a sequence of amino acids for which the chain length is sufficient to produce the higher levels of tertiary and/or quaternary structure. This is to distinguish from “peptides” that typically do not have such structure. Typically, the protein herein will have a molecular weight of at least about 15-100 kD, e.g., closer to about 15 kD. In certain embodiments, a protein can include at least about 50, about 60, about 70, about 80, about 90, about 100, about 200, about 300, about 400 or about 500 amino acids. Examples of proteins encompassed within the definition herein include all proteins, and, in general proteins that contain one or more disulfide bonds, including multi-chain polypeptides comprising one or more inter- and/or intrachain disulfide bonds. In certain embodiments, proteins can include other post-translation modifications including, but not limited to, glycosylation and lipidation. See, e.g., Prabakaran et al., WIREs Syst Biol Med (2012), which is incorporated herein by reference in its entirety.

The term “functional fragment thereof,” as used herein, refers to a fragment of a protein or peptide that retains at least a portion of the activity of the intact and/or full-length protein or peptide. In certain embodiments, the functional fragment retains at least about 10%, at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99% or at least about 100% of the activity of the intact and/or full-length protein.

The term “fragment thereof,” as used herein, refers to a fragment of a protein or peptide. In certain embodiments, the fragment comprises at least about 10%, at least about 20%, at least about 30%, at least about 40%, at least about 50%, at least about 60%, at least about 70%, at least about 80%, at least about 90%, at least about 91%, at least about 92%, at least about 93%, at least about 94%, at least about 95%, at least about 96%, at least about 97%, at least about 98%, at least about 99% or at least about 100% of the amino acids of the intact and/or full-length protein or peptide.

As used herein the terms “amino acid,” “amino acid monomer” or “amino acid residue” refer to organic compounds composed of amine and carboxylic acid functional groups, along with a side-chain specific to each amino acid. In particular, alpha- or α-amino acid refers to organic compounds in which the amine (—NH₂) is separated from the carboxylic acid (—COOH) by a methylene group (—CH₂), and a side-chain specific to each amino acid connected to this methylene group (—CH₂) which is alpha to the carboxylic acid (—COOH). Different amino acids have different side chains and have distinctive characteristics, such as charge, polarity, aromaticity, reduction potential, hydrophobicity and pKa. Amino acids can be covalently linked to form a polymer through peptide bonds by reactions between the carboxylic acid group of the first amino acid and the amine group of the second amino acid. Amino acid in the sense of the disclosure refers to any of the twenty plus naturally occurring amino acids, non-natural amino acids, and includes both D and L optical isomers.

The term “nucleic acid,” “nucleic acid molecule” or “polynucleotide” as used herein refers to any compound and/or substance that comprises a polymer of nucleotides. Each nucleotide is composed of a base, specifically a purine- or pyrimidine base (i.e., cytosine (C), guanine (G), adenine (A), thymine (T) or uracil (U)), a sugar (i.e., deoxyribose or ribose), and a phosphate group. Often, the nucleic acid molecule is described by the sequence of bases, whereby the bases represent the primary structure (linear structure) of a nucleic acid molecule. The sequence of bases is typically represented from 5′ to 3′. Herein, the term nucleic acid molecule encompasses deoxyribonucleic acid (DNA) including, e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule can be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of a nucleic acid of the disclosure in vitro and/or in vivo, e.g., in a yeast cell. Such DNA (e.g., cDNA) or RNA (e.g., mRNA) vectors can be unmodified or modified. For example, mRNA can be chemically modified to enhance the stability of the RNA vector and/or expression of the encoded molecule.

As used herein, the term “vector” refers to a nucleic acid molecule capable of transporting another nucleic acid to which it has been linked.

As used herein, the term “recombinant cell” refers to cells which have some genetic modification from the original parent cells from which they are derived. Such cells can also be referred to as “genetically-engineered cells.” Such genetic modification can be the result of an introduction of a heterologous gene (or nucleic acid) for expression of the gene product, e.g., a recombinant protein, e.g., a therapeutic.

As used herein, the term “recombinant protein” refers generally to peptides and proteins. Such recombinant proteins are “heterologous,” i.e., foreign to the cell being utilized, such as a heterologous secretory peptide produced by a yeast cell.

As used herein, “sequence identity” or “identity” in the context of two polynucleotide or polypeptide sequences makes reference to the nucleotide bases or amino acid residues in the two sequences that are the same when aligned for maximum correspondence over a specified comparison window. When percentage of sequence identity or similarity is used in reference to proteins, it is recognized that residue positions which are not identical often differ by conservative amino acid substitutions, where amino acid residues are substituted with a functionally equivalent residue of the amino acid residues with similar physiochemical properties and therefore do not change the functional properties of the molecule.

As used herein, the term “fusion protein” refers to a protein that includes all or a fragment of a protein that is linked, e.g., at the N-terminus or C-terminus, to a second protein or a fragment of the second protein. In certain embodiments, a first protein or fragment thereof is linked to a second protein or fragment thereof by a linker.

As would be understood by those skilled in the art, the term “codon optimization,” as used herein, refers to the introduction of synonymous mutations into codons of a protein-coding gene in order to improve protein expression in expression systems of a particular organism, such as a cell of a species of the phylum Ascomycota, in accordance with the codon usage bias of that organism. The term “codon usage bias” refers to differences in the frequency of occurrence of synonymous codons in coding DNA. The genetic codes of different organisms are often biased towards using one of the several codons that encode a same amino acid over others—thus using the one codon with, a greater frequency than expected by chance. Optimized codons in microorganisms, such as Saccharomyces cerevisiae, reflect the composition of their respective genomic tRNA pool. The use of optimized codons can help to achieve faster translation rates and high accuracy.

In the field of bioinformatics and computational biology, many statistical methods have been discussed and used to analyze codon usage bias. Methods such as the ‘frequency of optimal codons’ (Fop), the Relative Codon Adaptation (RCA) or the ‘Codon Adaptation Index’ (CAI) are used to predict gene expression levels, while methods such as the ‘effective number of codons’ (Nc) and Shannon entropy from information theory are used to measure codon usage evenness. Multivariate statistical methods, such as correspondence analysis and principal component analysis, are widely used to analyze variations in codon usage among genes. There are many computer programs to implement the statistical analyses enumerated above, including CodonW, GCUA, INCA, and others identifiable by those skilled in the art. Several software packages are available online for codon optimization of gene sequences, including those offered by companies such as GenScript, EnCor Biotechnology, Integrated DNA Technologies, ThermoFisher Scientific, among others known those skilled in the art. Those packages can be used in providing fusion protein genetic molecular components with codon ensuring optimized expression in assay systems as will be understood by a skilled person.

As used herein, “percentage of sequence identity” or “percentage of identity” means the value determined by comparing two optimally aligned sequences over a comparison window, wherein the portion of the polynucleotide sequence in the comparison window can include additions or deletions (gaps) as compared to the reference sequence (which does not include additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

As understood by those skilled in the art, determination of percent identity between any two sequences can be accomplished using certain well-known mathematical algorithms. Non-limiting examples of such mathematical algorithms are the algorithm of Myers and Miller, the local homology algorithm of Smith et al.; the homology alignment algorithm of Needleman and Wunsch; the search-for-similarity-method of Pearson and Lipman; the algorithm of Karlin and Altschul, modified as in Karlin and Altschul. Computer implementations of suitable mathematical algorithms can be utilized for comparison of sequences to determine sequence identity. Such implementations include, but are not limited to: CLUSTAL, ALIGN, GAP, BESTFIT, BLAST, FASTA, among others identifiable by skilled persons.

As used herein, “reference sequence” is a defined sequence used as a basis for sequence comparison. A reference sequence can be a subset or the entirety of a specified sequence; for example, as a segment of a full-length protein or protein fragment. A reference sequence can be, for example, a sequence identifiable in a database such as GenBank and UniProt and others identifiable to those skilled in the art.

The term “operative connection” or “operatively linked,” as used herein, with regard to regulatory sequences of a gene indicate an arrangement of elements in a combination enabling production of an appropriate effect. With respect to genes and regulatory sequences, an operative connection indicates a configuration of the genes with respect to the regulatory sequence allowing the regulatory sequences to directly or indirectly increase or decrease transcription or translation of the genes. In particular, in certain embodiments, regulatory sequences directly increasing transcription of the operatively linked gene, comprise promoters typically located on a same strand and upstream on a DNA sequence (towards the 5′ region of the sense strand), adjacent to the transcription start site of the genes whose transcription they initiate. In certain embodiments, regulatory sequences directly increasing transcription of the operatively linked gene or gene cluster comprise enhancers that can be located more distally from the transcription start site compared to promoters, and either upstream or downstream from the regulated genes, as understood by those skilled in the art. Enhancers are typically short (50-1500 bp) regions of DNA that can be bound by transcriptional activators to increase transcription of a particular gene. Typically, enhancers can be located up to 1 Mbp away from the gene, upstream or downstream from the start site.

The term “secretable,” as used herein, means able to be secreted, wherein secretion in the present disclosure generally refers to transport or translocation from the interior of a cell, e.g., within the cytoplasm or cytosol of a cell, to its exterior, e.g., outside the plasma membrane of the cell. Secretion can include several procedures, including various cellular processing procedures such as enzymatic processing of the peptide. In certain embodiments, secretion can utilize the classical secretory pathway of yeast.

The term “binding,” as used herein, refers to the connecting or uniting of two or more components by a interaction, bond, link, force or tie in order to keep two or more components together, which encompasses either direct or indirect binding where, for example, a first component is directly bound to a second component, or one or more intermediate molecules are disposed between the first component and the second component. Exemplary bonds comprise covalent bond, ionic bond, van der Waals interactions and other bonds identifiable by a skilled person. In certain embodiments, the binding can be direct, such as the production of a polypeptide scaffold that directly binds to a scaffold-binding element of a protein. In certain embodiments, the binding can be indirect, such as the co-localization of multiple protein elements on one scaffold. In certain embodiments, binding of a component with another component can result in sequestering the component, thus providing a type of inhibition of the component. In certain embodiments, binding of a component with another component can change the activity or function of the component, as in the case of allosteric or other interactions between proteins that result in conformational change of a component, thus providing a type of activation of the bound component.

The terms “detect” or “detection,” as used herein, indicates the determination of the existence and/or presence of a target in a limited portion of space, including but not limited to a sample, a reaction mixture, a molecular complex and a substrate. The “detect” or “detection” as used herein can comprise determination of chemical and/or biological properties of the target, including but not limited to ability to interact, and in particular bind, other compounds, ability to activate another compound and additional properties identifiable by a skilled person upon reading of the present disclosure. The detection can be quantitative or qualitative. A detection is “quantitative” when it refers, relates to, or involves the measurement of quantity or amount of the target or signal (also referred as quantitation), which includes but is not limited to any analysis designed to determine the amounts or proportions of the target or signal. A detection is “qualitative” when it refers, relates to, or involves identification of a quality or kind of the target or signal in terms of relative abundance to another target or signal, which is not quantified.

The term “derived” or “derive” is used herein to mean to obtain from a specified source.

The term “molecule,” as used herein, refers a group of atoms bonded together, representing the smallest fundamental unit of a chemical compound that can take part in a chemical reaction.

“Pharmaceutically acceptable carrier,” as used herein, refers to a pharmaceutically acceptable material, composition, or vehicle that is involved in carrying or transporting a compound or composition of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

As used herein the term “subject” refers to any animal (e.g., a mammal), including, but not limited to, humans, non-human primates, rodents, and the like, which is to be recipient of a yeast-based vaccine described herein.

As used herein, the term “adjuvant” refers to a nonspecific stimulant of the immune response. The adjuvant can be (a) a substance designed to form a deposit protecting the antigen(s) from rapid catabolism (e.g., mineral oil, alum, aluminum hydroxide, liposome or surfactant) and/or (b) a substance that nonspecifically stimulates the immune response of the immunized subject (e.g., by increasing lymphokine levels).

As used herein, the term “cell targeting molecule” refers to a molecule, e.g., a peptide or protein or fragment thereof, that targets the antigen and/or the genetically-engineered cell secreting the antigen to a particular cell type or tissue, e.g., an immune cell and/or a mucosal tissue. For example, but not by way of limitation, a cell targeting molecule can target the antigen and/or the genetically-engineered cell secreting the antigen to mucosal immune tissues, e.g., Peyer's patches in the small intestines.

As used herein, the terms “conservative amino acid substitutions” and “conservative modifications” refer to amino acid modifications that do not significantly affect or alter the function and/or activity of the presently disclosed proteins comprising the amino acid sequence. Such conservative modifications include amino acid substitutions, additions and deletions. Modifications can be introduced into the proteins of this disclosure by standard techniques known in the art, such as site-directed mutagenesis and PCR-mediated mutagenesis. Amino acids can be classified into groups according to their physicochemical properties such as charge and polarity.

Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. In certain embodiments, no more than one, no more than two, no more than three, no more than four, no more than five residues within a specified sequence are altered. Exemplary conservative amino acid substitutions are shown in Table 1 below.

TABLE 1

| Original Residue | Exemplary Conservative Amino Acid Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gin (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe |
| Leu (L) | Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala |

### II. Antigen

The present disclosure provides cells that express and/or secrete one or more antigens derived from a virus. For example, but not by way of limitation, a cell, e.g., a genetically-engineered cell, of the present disclosure can produce and/or secrete an antigen derived from a coronavirus.

In certain embodiments, a cell, e.g., a genetically-engineered cell, of the present disclosure can produce and/or secrete more than one antigen, e.g., two antigens, three antigens, four antigens or five antigens or more derived from a coronavirus. In certain embodiments, a cell, e.g., a genetically-engineered cell, of the present disclosure can produce and/or secrete more than one antigen, e.g., two antigens, three antigens, four antigens or five antigens or more derived from SARS-CoV-2. In certain embodiments, a cell, e.g., a genetically-engineered cell, of the present disclosure can produce and/or secrete more than one antigen, e.g., two antigens, three antigens, four antigens or five antigens or more, derived from a single coronavirus strain or variant, derived from more than one coronavirus strain or derived from more than one coronavirus variant.

In certain embodiments, a multi-cell system can be used for the generation and secretion of multiple different antigens derived from of a coronavirus. In certain embodiments, a multi-cell system can be used for the generation and secretion of 2, 3, 4, 5, 6, 7, 8, 9 or 10 different antigens of a single coronavirus strain or variant, of more than one coronavirus strain or of more than one coronavirus variant. Non-limiting examples of such multi-cell systems are disclosed PCT/US2020/030795, the contents of which is incorporated herein in its entirety.

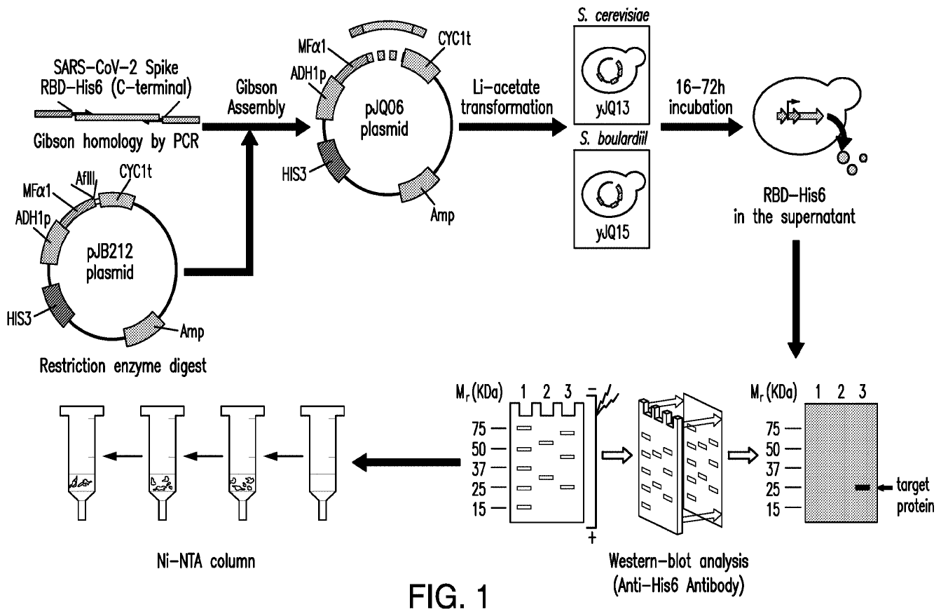

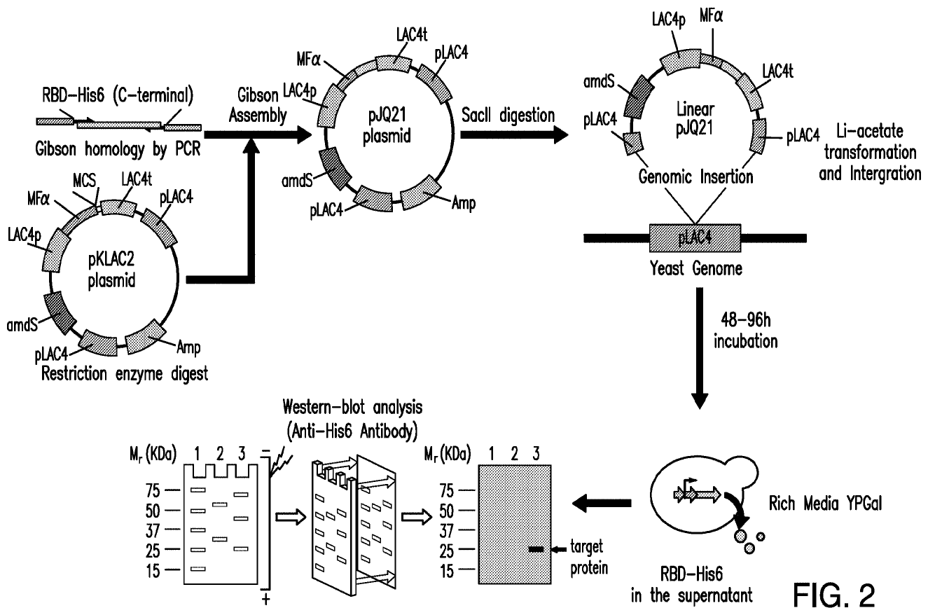

In certain embodiments, secretion can be performed using the conserved secretory pathway in fungal cells, e.g., yeast. For example, but not by way of limitation, an antigen is secretable because it is coupled to a secretion signal sequence. Examples of secretion signal sequences can be obtained from proteins including mating factor alpha-1, alpha factor K, alpha factor T, glycoamylase, inulinase, invertase, lysozyme, serum albumin, alpha-amylase and killer protein. In certain embodiments, the secretion signal sequence is a secretion signal sequence obtained from a yeast protein, such as a Saccharomyces cerevisiae protein. In certain embodiments, the secretion signal peptide is obtained from the Saccharomyces cerevisiae mating factor alpha-1 (MFα1) or Kluyveromyces lactis mating factor alpha (MFα). See FIGS. 1 and 2. Additionally, mutations, substitutions and truncations of any signal peptide are also within the scope of the present disclosure. The selection and design, including additional mutations and truncations of a signal peptide is within the ability and discretion of one of ordinary skill in the art. In certain embodiments, the one or more secretion signal sequences are located at the N-terminus of a secretable peptide. In certain embodiments, a Kex2 processing site and/or a Ste13 processing site or a homolog thereof can be present between the amino acid sequence of the secretion signal sequence and the secretable antigen. Additional non-limiting examples of secretion signals are disclosed in U.S. Pat. No. 10,725,036, the contents of which is disclosed herein in its entirety. In certain embodiments, an antigen of the present disclosure is not displayed on the surface of a genetically-engineered cell.

In certain embodiments, the coronavirus is an alpha-coronavirus, a beta-coronavirus, a gamma-coronavirus or a delta-coronavirus. In certain embodiments, the coronavirus is an alpha-coronavirus. In certain embodiments, the coronavirus is a beta-coronavirus. In certain embodiments, the coronavirus is a gamma-coronavirus. In certain embodiments, the coronavirus is a delta-coronavirus.

In certain embodiments, the coronavirus is an alpha-coronavirus such as, but not limited to, the coronavirus strains HCoV-229E and HCoV-NL63.

In certain embodiments, the coronavirus is a beta-coronavirus such as, but not limited to, the coronavirus strains HCoV-0C43, HCoV-HKU1, MERS-CoV (which causes Middle East Respiratory Syndrome or MERS) and SARS-CoV (which causes severe acute respiratory syndrome or SARS). In certain embodiments, the coronavirus is MERS-CoV. In certain embodiments, the coronavirus is SARS-CoV.

In certain embodiments, the coronavirus is SARS-CoV-2 (which causes coronavirus disease 2019 or COVID-19). In certain embodiments, the coronavirus can be a variant of SARS-CoV-2. In certain embodiments, the SARS-CoV-2 variant can be an alpha variant, a beta variant, a gamma variant, an epsilon variant, a kappa variant, an iota variant, an eta variant, a lambda variant or a zeta variant. Non-limiting examples of SARS-CoV-2 variants include the B.1.1.7 (alpha), B.1.351 (beta), P.1 (gamma), B.1.427 (epsilon), B.1.429 (epsilon), B.1.617.2 (delta), B.1.526.1 (iota), B.1.526.2 (iota), B.1.525 (eta), P.2 (zeta) and B.1.526 (iota) variants. In certain embodiments, the coronavirus is the SARS-CoV-2 B.1.1.7 variant. In certain embodiments, the coronavirus is the SARS-CoV-2 B.1.351 variant. In certain embodiments, the coronavirus is the SARS-CoV-2 P.1 variant. In certain embodiments, the coronavirus is the SARS-CoV-2 B.1.427 variant. In certain embodiments, the coronavirus is the SARS-CoV-2 B.1.429 variant. In certain embodiments, the coronavirus is the SARS-CoV-2 B.1.617.2 variant.

In certain embodiments, the antigen comprises a peptide derived from a coronavirus, e.g., SARS-CoV-2. For example, but not by way of limitation, the antigen can comprise a peptide derived from a coronavirus that has a length of 3 residues or more, a length of 4 residues or more, a length of 5 residues or more, 6 residues or more, 7, residues or more, 8 residues or more, 9 residues or more, 10 residues or more, 11 residues or more, 12 residues or more, 13 residues or more, 14 residues or more, 15 residues or more, 16 residues or more, 17 residues or more, 18 residues or more, 19 residues or more, 20 residues or more, 21 residues or more, 22 residues or more, 23 residues or more, 24 residues or more, 25 residues or more, 26 residues or more, 27 residues or more, 28 residues or more, 29 residues or more, 30 residues or more, 31 residues or more, 32 residues or more, 33 residues or more, 34 residues or more, 35 residues or more, 36 residues or more, 37 residues or more, 38 residues or more, 39 residues or more, 40 residues or more, 41 residues or more, 42 residues or more, 43 residues or more, 44 residues or more, 45 residues or more, 46 residues or more, 47 residues or more, 48 residues or more, 49 residues or more or 50 residues or more. In certain embodiments, the antigen has a length of 3-50 residues, 5-50 residues, 3-45 residues, 5-45 residues, 3-40 residues, 5-40 residues, 3-35 residues, 5-35 residues, 3-30 residues, 5-30 residues, 3-25 residues, 5-25 residues, 3-20 residues, 5-20 residues, 3-15 residues, 5-15 residues, 3-10 residues, 3-10 residues, 5-10 residues, 10-15 residues, 15-20 residues, 20-25 residues, 25-30 residues, 30-35 residues, 35-40 residues, 40-45 residues or 45-50 residues. In certain embodiments, the antigen has a length of about 5 to about 30 residues.

In certain embodiments, the antigen comprises a protein or fragment thereof derived from coronavirus, e.g., SARS-CoV-2. In certain embodiments, the antigen comprises a spike (S) protein or fragment thereof from a coronavirus, e.g., SARS-CoV-2. Non-limiting examples of an S protein or fragment thereof include the S1 subunit, the S2 subunit and the prefusion S protein. In certain embodiments, the antigen comprises the S1 subunit or a fragment thereof. In certain embodiments, a fragment of an S protein for use as an antigen can comprise a domain and/or region of the S protein or fragment thereof. Non-limiting examples of regions of the S protein include the N-terminal domain (NTD), the receptor-binding domain (RBD) and the RBD with spike domain 1 (RBD-SD1) region.

In certain embodiments, an S protein for use as an antigen comprises the amino acid sequence of GenBank Accession No. 43740568. In certain embodiments, an S protein for use as an antigen is encoded by a nucleotide sequence comprising the sequence of GenBank Accession No. 43740568.

In certain embodiments, an S protein for use as an antigen can comprise or consist of an amino acid sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the amino acid sequence set forth in SEQ ID NO: 12. In certain embodiments, an S protein for use as an antigen can comprise or consist of the amino acid sequence of SEQ ID NO: 12. In certain embodiments, an S protein for use as an antigen can be encoded by a nucleotide sequence that comprises or consists of a nucleotide sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the nucleotide sequence set forth in SEQ ID NO: 1. In certain embodiments, an S protein for use as an antigen can be encoded by a nucleotide sequence that comprises or consists of the nucleotide sequence of SEQ ID NO: 1.

In certain embodiments, the S protein or fragment thereof is a mutated version of an S protein or fragment thereof. In certain embodiments, the mutated S protein has one or more amino acid substitutions. For example, but not by way of limitation, the mutated S protein has one or more amino acid substitutions to reduce the glycosylation and/or disulfide bond formation of the S protein, e.g., the RBD of the S protein. Non-limiting examples of such substitutions are disclosed in Table 4. The amino acid residues at positions 331 and 343 are responsible for N-linked glycosylation, and the amino residue at position 538 is responsible for disulfide bond formation. Without being limited to a particular theory, mutations at these residues increase the stability of the antigen, e.g., RBD. In certain embodiments, a mutated S protein or fragment thereof has a substitution at one or more amino acid positions selected from the amino acid residues at positions 331, 343, 538 and a combination thereof. In certain embodiments, the amino acid residue that is mutated is at position 331. In certain embodiments, the amino acid residue that is mutated is at position 343. In certain embodiments, the amino acid residue that is mutated is at position 538. In certain embodiments, the S protein or fragment thereof is mutated at amino acid residues 331 and 343. In certain embodiments, the spike protein or fragment thereof is mutated at amino acid residues 331 and 538. In certain embodiments, the spike protein or fragment thereof is mutated at amino acid residues 343 and 538. In certain embodiments, amino acid residue 331 is mutated to N331A. In certain embodiments, amino acid residue 343 is mutated to N343A. In certain embodiments, amino acid residue 538 is mutated to C538A.

In certain embodiments, the antigen comprises a fragment of an S protein. For example, but not by way of limitation, a fragment of an S protein for use as an antigen comprises or consists of about 100-500 consecutive amino acids of the S protein. In certain embodiments, a fragment of an S protein comprises or consists of about 200 to about 500, about 250 to about 500, about 300 to about 500, about 100 to about 450, about 100 to about 400, about 100 to about 350, about 100 to about 300, about 100 to about 250, about 200 to about 400 or about 250 to about 350 consecutive amino acids of the S protein. In certain embodiments, the fragment of an S protein for use as an antigen comprises or consists of about 250 to about 350 consecutive amino acids of the S protein.

In certain embodiments, the antigen is an NTD of an S protein, e.g., an S protein from SARS-CoV-2. In certain embodiments, the NTD comprises or consists of amino acids 14-305 of the S protein. Non-limiting examples of an NTD for use as an antigen are disclosed in Table 2 and Zhou et al., Cell Rep. 33:108322 (2020), the contents of which are incorporated by reference herein.

In certain embodiments, the antigen is an RBD-SD1 of an S protein, e.g., an S protein from SARS-CoV-2. In certain embodiments, the RBD-SD1 comprises or consists of amino acids 319-591 of the S protein. Non-limiting examples of an RBD-SD1 for use as an antigen are disclosed in Table 2 and Zhou et al., Cell Rep. 33:108322 (2020), the contents of which are incorporated by reference herein.

In certain embodiments, the antigen is an RBD of an S protein, e.g., an S protein from SARS-CoV-2. In certain embodiments, the RBD comprises or consists of amino acids 319-541 of the S protein. In certain embodiments, the RBD comprises or consists of amino acids 329-526 of the S protein. Non-limiting examples of RBD sequences for use as an antigen are disclosed in Table 2 and Zhou et al., Cell Rep. 33:108322 (2020), the contents of which are incorporated by reference herein.

In certain embodiments, the antigen comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a sequence disclosed in Table 2. In certain embodiments, the antigen comprises an amino acid sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to an amino acid sequence set forth in SEQ ID NOs: 12-22. In certain embodiments, the antigen comprises an amino acid sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the amino acid sequence set forth in SEQ ID NO: 12. In certain embodiments, the antigen comprises an amino acid sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the amino acid sequence set forth in SEQ ID NO: 13. In certain embodiments, the antigen comprises an amino acid sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the amino acid sequence set forth in SEQ ID NO: 14. In certain embodiments, the antigen comprises an amino acid sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the amino acid sequence set forth in SEQ ID NO: 15. In certain embodiments, the antigen comprises an amino acid sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the amino acid sequence set forth in SEQ ID NO: 16. In certain embodiments, the antigen comprises an amino acid sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the amino acid sequence set forth in SEQ ID NO: 17. In certain embodiments, the antigen comprises an amino acid sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the amino acid sequence set forth in SEQ ID NO: 18. In certain embodiments, the antigen comprises an amino acid sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the amino acid sequence set forth in SEQ ID NO: 19. In certain embodiments, the antigen comprises an amino acid sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the amino acid sequence set forth in SEQ ID NO: 20. In certain embodiments, the antigen comprises an amino acid sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the amino acid sequence set forth in SEQ ID NO: 21. In certain embodiments, the antigen comprises an amino acid sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the amino acid sequence set forth in SEQ ID NO: 22.

In certain embodiments, the antigen comprises an amino acid sequence set forth in Table 2 or any one of the sequences disclosed in Table 2 bearing about 1 or about 2 amino acid substitutions, insertions or deletions, or about 1, about 2 or about 3 conservative amino acid substitutions. In certain embodiments, the antigen comprises an amino acid sequence set forth in SEQ ID NOs: 12-22 or any one of the sequences of SEQ ID NOs: 12-22 bearing about 1 or about 2 amino acid substitutions, insertions or deletions, or about 1, about 2 or about 3 conservative amino acid substitutions.

In certain embodiments, the antigen comprises an amino acid sequence set forth in Table 2. In certain embodiments, the antigen consists of an amino acid sequence set forth in Table 2. In certain embodiments, the antigen comprises an amino acid sequence set forth in SEQ ID NOs: 12-22. In certain embodiments, the antigen consists of an amino acid sequence set forth in SEQ ID NOs: 12-22. In certain embodiments, the antigen comprises or consists of the amino acid sequence set forth in SEQ ID NO: 12. In certain embodiments, the antigen comprises or consists of the amino acid sequence set forth in SEQ ID NO: 13. In certain embodiments, the antigen comprises or consists of the amino acid sequence set forth in SEQ ID NO: 14. In certain embodiments, the antigen comprises or consists of the amino acid sequence set forth in SEQ ID NO: 15. In certain embodiments, the antigen comprises or consists of the amino acid sequence set forth in SEQ ID NO: 16. In certain embodiments, the antigen comprises or consists of the amino acid sequence set forth in SEQ ID NO: 17. In certain embodiments, the antigen comprises or consists of the amino acid sequence set forth in SEQ ID NO: 18. In certain embodiments, the antigen comprises or consists of an amino acid sequence set forth in SEQ ID NO: 19. In certain embodiments, the antigen comprises or consists of the amino acid sequence set forth in SEQ ID NO: 20. In certain embodiments, the antigen comprises or consists of the amino acid sequence set forth in SEQ ID NO: 21. In certain embodiments, the antigen comprises or consists of the amino acid sequence set forth in SEQ ID NO: 22.

In certain embodiments, the antigen can be encoded by a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a sequence disclosed in Table 2. In certain embodiments, the antigen can be encoded by a nucleotide sequence that is at least about 40%, at least about 50%, at least about 60%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% homologous to a nucleotide sequence set forth in SEQ ID NOs: 1-11. In certain embodiments, the antigen can be encoded by a nucleotide sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the nucleotide sequence set forth in SEQ ID NO: 1. In certain embodiments, the antigen can be encoded by a nucleotide sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the nucleotide sequence set forth in SEQ ID NO: 2. In certain embodiments, the antigen can be encoded by a nucleotide sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the nucleotide sequence set forth in SEQ ID NO: 3. In certain embodiments, the antigen can be encoded by a nucleotide sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the nucleotide sequence set forth in SEQ ID NO: 4. In certain embodiments, the antigen can be encoded by a nucleotide sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the nucleotide sequence set forth in SEQ ID NO: 5. In certain embodiments, the antigen can be encoded by a nucleotide sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the nucleotide sequence set forth in SEQ ID NO: 6. In certain embodiments, the antigen can be encoded by a nucleotide sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the nucleotide sequence set forth in SEQ ID NO: 7. In certain embodiments, the antigen can be encoded by a nucleotide sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the nucleotide sequence set forth in SEQ ID NO: 8. In certain embodiments, the antigen can be encoded by a nucleotide sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the nucleotide sequence set forth in SEQ ID NO: 9. In certain embodiments, the antigen can be encoded by a nucleotide sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the nucleotide sequence set forth in SEQ ID NO: 10. In certain embodiments, the antigen can be encoded by a nucleotide sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the nucleotide sequence set forth in SEQ ID NO: 11.

In certain embodiments, the antigen comprises a nucleotide sequence set forth in Table 2 or any one of the sequences disclosed in Table 2 bearing about 1, about 2 or about 3 nucleotide substitutions, insertions or deletions, or about 1, about 2 or about 3 conservative nucleotide substitutions. In certain embodiments, the antigen comprises a nucleotide sequence set forth in SEQ ID NOs: 1-11 or any one of the sequences of SEQ ID NOs: 1-11 bearing about 1, about 2 or about 3 nucleotide substitutions, insertions or deletions, or about 1, about 2 or about 3 conservative nucleotide substitutions.

In certain embodiments, the antigen comprises a nucleotide sequence set forth in Table 2. In certain embodiments, the antigen consists of a nucleotide sequence set forth in Table 2. In certain embodiments, the antigen comprises a nucleotide sequence set forth in SEQ ID NOs: 1-11. In certain embodiments, the antigen consists of a nucleotide sequence set forth in SEQ ID NOs: 1-11. In certain embodiments, the antigen comprises or consists of the nucleotide sequence set forth in SEQ ID NO: 1. In certain embodiments, the antigen comprises or consists of the nucleotide sequence set forth in SEQ ID NO: 2. In certain embodiments, the antigen comprises or consists of the nucleotide sequence set forth in SEQ ID NO: 3. In certain embodiments, the antigen comprises or consists of the nucleotide sequence set forth in SEQ ID NO: 4. In certain embodiments, the antigen comprises or consists of the nucleotide sequence set forth in SEQ ID NO: 5. In certain embodiments, the antigen comprises or consists of the nucleotide sequence set forth in SEQ ID NO: 6. In certain embodiments, the antigen comprises or consists of the nucleotide sequence set forth in SEQ ID NO: 7. In certain embodiments, the antigen comprises or consists of the nucleotide sequence set forth in SEQ ID NO: 8. In certain embodiments, the antigen comprises or consists of the nucleotide sequence set forth in SEQ ID NO: 9. In certain embodiments, the antigen comprises or consists of the nucleotide sequence set forth in SEQ ID NO: 10.

TABLE 2

| Antigen | Nucleotide Sequence | Amino Acid Sequence | Source |
|---|---|---|---|
| Spike | ATGTTTGTTTTTCTTGTTTTATTGCC | MFVFLVLLPLVSSQCVNLTTRTQ | GenBank |
| Protein | ACTAGTCTCTAGTCAGTGTGTTAAT | LPPAYTNSFTRGVYYPDKVFRSS | Accession |
| | CTTACAACCAGAACTCAATTACCC | VLHSTQDLFLPFFSNVTWFHAIH | No. |
| | CCTGCATACACTAATTCTTTCACAC | VSGTNGTKRFDNPVLPFNDGVY | 43740568 |
| | GTGGTGTTTATTACCCTGACAAAGT | FASTEKSNIIRGWIFGTTLDSKTQ | |
| | TTTCAGATCCTCAGTTTTACATTCA | SLLIVNNATNVVIKVCEFQFCND | |
| | ACTCAGGACTTGTTCTTACCTTTCT | PFLGVYYHKNNKSWMESEFRVY | |
| | TTTCCAATGTTACTTGGTTCCATGC | SSANNCTFEYVSQPFLMDLEGK | |
| | TATACATGTCTCTGGGACCAATGGT | QGNFKNLREFVFKNIDGYFKIYS | |
| | ACTAAGAGGTTTGATAACCCTGTC | KHTPINLVRDLPQGFSALEPLVD | |
| | CTACCATTTAATGATGGTGTTTATT | LPIGINITRFQTLLALHRSYLTPG | |
| | TTGCTTCCACTGAGAAGTCTAACAT | DSSSGWTAGAAAYYVGYLQPRT | |
| | AATAAGAGGCTGGATTTTTGGTAC | FLLKYNENGTITDAVDCALDPLS | |
| | TACTTTAGATTCGAAGACCCAGTCC | ETKCTLKSFTVEKGIYQTSNFRV | |
| | CTACTTATTGTTAATAACGCTACTA | QPTESIVRFPNITNLCPFGEVFNA | |
| | ATGTTGTTATTAAAGTCTGTGAATT | TRFASVYAWNRKRISNCVADYS | |
| | TCAATTTTGTAATGATCCATTTTTG | VLYNSASFSTFKCYGVSPTKLND | |
| | GGTGTTTATTACCACAAAAACAAC | LCFTNVYADSFVIRGDEVRQIAP | |
| | AAAAGTTGGATGGAAAGTGAGTTC | GQTGKIADYNYKLPDDFTGCVIA | |
| | AGAGTTTATTCTAGTGCGAATAATT | WNSNNLDSKVGGNYNYLYRLF | |
| | GCACTTTTGAATATGTCTCTCAGCC | RKSNLKPFERDISTEIYQAGSTPC | |
| | TTTTCTTATGGACCTTGAAGGAAAA | NGVEGFNCYFPLQSYGFQPTNG | |
| | CAGGGTAATTTCAAAAATCTTAGG | VGYQPYRVVVLSFELLHAPATV | |
| | GAATTTGTGTTTAAGAATATTGATG | CGPKKSTNLVKNKCVNFNFNGL | |
| | GTTATTTTAAAATATATTCTAAGCA | TGTGVLTESNKKFLPFQQFGRDI | |
| | CACGCCTATTAATTTAGTGCGTGAT | ADTTDAVRDPQTLEILDITPCSFG | |
| | CTCCCTCAGGGTTTTTCGGCTTTAG | GVSVITPGTNTSNQVAVLYQDV | |
| | AACCATTGGTAGATTTGCCAATAG | NCTEVPVAIHADQLTPTWRVYS | |
| | GTATTAACATCACTAGGTTTCAAAC | TGSNVFQTRAGCLIGAEHVNNS | |
| | TTTACTTGCTTTACATAGAAGTTAT | YECDIPIGAGICASYQTQTNSPRR | |
| | TTGACTCCTGGTGATTCTTCTTCAG | ARSVASQSIIAYTMSLGAENSVA | |
| | GTTGGACAGCTGGTGCTGCAGCTT | YSNNSIAIPTNFTISVTTEILPVSM | |
| | ATTATGTGGGTTATCTTCAACCTAG | TKTSVDCTMYICGDSTECSNLLL | |
| | GACTTTTCTATTAAAATATAATGAA | QYGSFCTQLNRALTGIAVEQDK | |
| | AATGGAACCATTACAGATGCTGTA | NTQEVFAQVKQIYKTPPIKDFGG | |
| | GACTGTGCACTTGACCCTCTCTCAG | FNFSQILPDPSKPSKRSFIEDLLFN | |
| | AAACAAAGTGTACGTTGAAATCCT | KVTLADAGFIKQYGDCLGDIAA | |
| | TCACTGTAGAAAAAGGAATCTATC | RDLICAQKFNGLTVLPPLLTDEM | |
| | AAACTTCTAACTTTAGAGTCCAACC | IAQYTSALLAGTITSGWTFGAGA | |
| | AACAGAATCTATTGTTAGATTTCCT | ALQIPFAMQMAYRFNGIGVTQN | |
| | AATATTACAAACTTGTGCCCTTTTG | VLYENQKLIANQFNSAIGKIQDS | |
| | GTGAAGTTTTTAACGCCACCAGATT | LSSTASALGKLQDVVNQNAQAL | |
| | TGCATCTGTTTATGCTTGGAACAGG | NTLVKQLSSNFGAISSVLNDILSR | |
| | AAGAGAATCAGCAACTGTGTTGCT | LDKVEAEVQIDRLITGRLQSLQT | |
| | GATTATTCTGTCCTATATAATTCCG | YVTQQLIRAAEIRASANLAATK | |
| | CATCATTTTCCACTTTTAAGTGTTA | MSECVLGQSKRVDFCGKGYHL | |
| | TGGAGTGTCTCCTACTAAATTAAAT | MSFPQSAPHGVVFLHVTYVPAQ | |
| | GATCTCTGCTTTACTAATGTCTATG | EKNFTTAPAICHDGKAHFPREGV | |
| | CAGATTCATTTGTAATTAGAGGTG | FVSNGTHWFVTQRNFYEPQIITT | |
| | ATGAAGTCAGACAAATCGCTCCAG | DNTFVSGNCDVVIGIVNNTVYDP | |
| | GGCAAACTGGAAAGATTGCTGATT | LQPELDSFKEELDKYFKNHTSPD | |
| | ATAATTATAAATTACCAGATGATTT | VDLGDISGINASVVNIQKEIDRLN | |
| | TACAGGCTGCGTTATAGCTTGGAA | EVAKNLNESLIDLQELGKYEQYI | |
| | TTCTAACAATCTTGATTCTAAGGTT | KWPWYIWLGFIAGLIAIVMVTIM | |
| | GGTGGTAATTATAATTACCTGTATA | LCCMTSCCSCLKGCCSCGSCCKF | |
| | GATTGTTTAGGAAGTCTAATCTCAA | DEDDSEPVLKGVKLHYT | |
| | ACCTTTTGAGAGAGATATTTCAACT | (SEQ ID NO: 12) | |
| | GAAATCTATCAGGCCGGTAGCACA | | |
| | CCTTGTAATGGTGTTGAAGGTTTTA | | |
| | ATTGTTACTTTCCTTTACAATCATA | | |
| | TGGTTTCCAACCCACTAATGGTGTT | | |
| | GGTTACCAACCATACAGAGTAGTA | | |
| | GTACTTTCTTTTGAACTTCTACATG | | |
| | CACCAGCAACTGTTTGTGGACCTA | | |
| | AAAAGTCTACTAATTTGGTTAAAA | | |
| | ACAAATGTGTCAATTTCAACTTCAA | | |
| | TGGTTTAACAGGCACAGGTGTTCTT | | |
| | ACTGAGTCTAACAAAAAGTTTCTG | | |
| | CCTTTCCAACAATTTGGCAGAGAC | | |
| | ATTGCTGACACTACTGATGCTGTCC | | |
| | GTGATCCACAGACACTTGAGATTC | | |
| | TTGACATTACACCATGTTCTTTTGG | | |
| | TGGTGTCAGTGTTATAACACCAGG | | |
| | AACAAATACTTCTAACCAGGTTGC | | |
| | TGTTCTTTATCAGGATGTTAACTGC | | |
| | ACAGAAGTCCCTGTTGCTATTCATG | | |
| | CAGATCAACTTACTCCTACTTGGCG | | |
| | TGTTTATTCTACAGGTTCTAATGTT | | |
| | TTTCAAACACGTGCAGGCTGTTTAA | | |
| | TAGGGGCTGAACATGTCAACAACT | | |
| | CATATGAGTGTGACATACCCATTG | | |
| | GTGCAGGTATATGCGCTAGTTATC | | |
| | AGACTCAGACTAATTCTCCTCGGC | | |
| | GGGCACGTAGTGTAGCTAGTCAAT | | |
| | CCATCATTGCCTACACTATGTCACT | | |
| | TGGTGCAGAAAATTCAGTTGCTTA | | |
| | CTCTAATAACTCTATTGCCATACCC | | |
| | ACAAATTTTACTATTAGTGTTACCA | | |
| | CAGAAATTCTACCAGTGTCTATGA | | |
| | CCAAGACATCAGTAGATTGTACAA | | |
| | TGTACATTTGTGGTGATTCAACTGA | | |
| | ATGCAGCAATCTTTTGTTGCAATAT | | |
| | GGCAGTTTTTGTACACAATTAAACC | | |
| | GTGCTTTAACTGGAATAGCTGTTGA | | |
| | ACAAGACAAAAACACCCAAGAAGT | | |
| | TTTTGCACAAGTCAAACAAATTTAC | | |
| | AAAACACCACCAATTAAAGATTTT | | |
| | GGTGGTTTTAATTTTTCACAAATAT | | |
| | TACCAGATCCATCAAAACCAAGCA | | |
| | AGAGGTCATTTATTGAAGATCTACT | | |
| | TTTCAACAAAGTGACACTTGCAGA | | |
| | TGCTGGCTTCATCAAACAATATGGT | | |
| | GATTGCCTTGGTGATATTGCTGCTA | | |
| | GAGACCTCATTTGTGCACAAAAGT | | |
| | TTAACGGCCTTACTGTTTTGCCACC | | |
| | TTTGCTCACAGATGAAATGATTGCT | | |
| | CAATACACTTCTGCACTGTTAGCGG | | |
| | GTACAATCACTTCTGGTTGGACCTT | | |
| | TGGTGCAGGTGCTGCATTACAAAT | | |
| | ACCATTTGCTATGCAAATGGCTTAT | | |
| | AGGTTTAATGGTATTGGAGTTACA | | |
| | CAGAATGTTCTCTATGAGAACCAA | | |
| | AAATTGATTGCCAACCAATTTAAT | | |
| | AGTGCTATTGGCAAAATTCAAGAC | | |
| | TCACTTTCTTCCACAGCAAGTGCAC | | |
| | TTGGAAAACTTCAAGATGTGGTCA | | |
| | ACCAAAATGCACAAGCTTTAAACA | | |
| | CGCTTGTTAAACAACTTAGCTCCAA | | |
| | TTTTGGTGCAATTTCAAGTGTTTTA | | |
| | AATGATATCCTTTCACGTCTTGACA | | |
| | AAGTTGAGGCTGAAGTGCAAATTG | | |
| | ATAGGTTGATCACAGGCAGACTTC | | |
| | AAAGTTTGCAGACATATGTGACTC | | |
| | AACAATTAATTAGAGCTGCAGAAA | | |
| | TCAGAGCTTCTGCTAATCTTGCTGC | | |
| | TACTAAAATGTCAGAGTGTGTACTT | | |
| | GGACAATCAAAAAGAGTTGATTTT | | |
| | TGTGGAAAGGGCTATCATCTTATGT | | |
| | CCTTCCCTCAGTCAGCACCTCATGG | | |
| | TGTAGTCTTCTTGCATGTGACTTAT | | |
| | GTCCCTGCACAAGAAAAGAACTTC | | |
| | ACAACTGCTCCTGCCATTTGTCATG | | |
| | ATGGAAAAGCACACTTTCCTCGTG | | |
| | AAGGTGTCTTTGTTTCAAATGGCAC | | |
| | ACACTGGTTTGTAACACAAAGGAA | | |
| | TTTTTATGAACCACAAATCATTACT | | |
| | ACAGACAACACATTTGTGTCTGGT | | |
| | AACTGTGATGTTGTAATAGGAATT | | |
| | GTCAACAACACAGTTTATGATCCTT | | |
| | TGCAACCTGAATTAGACTCATTCA | | |
| | AGGAGGAGTTAGATAAATATTTTA | | |
| | AGAATCATACATCACCAGATGTTG | | |
| | ATTTAGGTGACATCTCTGGCATTAA | | |
| | TGCTTCAGTTGTAAACATTCAAAA | | |
| | AGAAATTGACCGCCTCAATGAGGT | | |
| | TGCCAAGAATTTAAATGAATCTCTC | | |
| | ATCGATCTCCAAGAACTTGGAAAG | | |
| | TATGAGCAGTATATAAAATGGCCA | | |
| | TGGTACATTTGGCTAGGTTTTATAG | | |
| | CTGGCTTGATTGCCATAGTAATGGT | | |
| | GACAATTATGCTTTGCTGTATGACC | | |
| | AGTTGCTGTAGTTGTCTCAAGGGCT | | |
| | GTTGTTCTTGTGGATCCTGCTGCAA | | |
| | ATTTGATGAAGACGACTCTGAGCC | | |
| | AGTGCTCAAAGGAGTCAAATTACA | | |
| | TTACACATAA (SEQ ID NO: 1) | | |
| NTD | CAGTGTGTTAATCTTACAACCAGA | QCVNLTTRTQLPPAYTNSFTRGV | Zhou et al., |
| (aa 14-305) | ACTCAATTACCCCCTGCATACACTA | YYPDKVFRSSVLHSTQDLFLPFF | Cell Rep. |
| | ATTCTTTCACACGTGGTGTTTATTA | SNVTWFHAIHVSGTNGTKRFDN | 33: 108322 |
| | CCCTGACAAAGTTTTCAGATCCTCA | PVLPFNDGVYFASTEKSNIIRGWI | (2020) |
| | GTTTTACATTCAACTCAGGACTTGT | FGTTLDSKTQSLLIVNNATNVVI | |
| | TCTTACCTTTCTTTTCCAATGTTACT | KVCEFQFCNDPFLGVYYHKNNK | |
| | TGGTTCCATGCTATACATGTCTCTG | SWMESEFRVYSSANNCTFEYVS | |
| | GGACCAATGGTACTAAGAGGTTTG | QPFLMDLEGKQGNFKNLREFVF | |
| | ATAACCCTGTCCTACCATTTAATGA | KNIDGYFKIYSKHTPINLVRDLPQ | |
| | TGGTGTTTATTTTGCTTCCACTGAG | GFSALEPLVDLPIGINITRFQTLLA | |
| | AAGTCTAACATAATAAGAGGCTGG | LHRSYLTPGDSSSGWTAGAAAY | |
| | ATTTTTGGTACTACTTTAGATTCGA | YVGYLQPRTFLLKYNENGTITDA | |
| | AGACCCAGTCCCTACTTATTGTTAA | VDCALDPLSETKCTLKS | |
| | TAACGCTACTAATGTTGTTATTAAA | (SEQ ID NO: 13) | |
| | GTCTGTGAATTTCAATTTTGTAATG | | |
| | ATCCATTTTTGGGTGTTTATTACCA | | |
| | CAAAAACAACAAAAGTTGGATGGA | | |
| | AAGTGAGTTCAGAGTTTATTCTAGT | | |
| | GCGAATAATTGCACTTTTGAATATG | | |
| | TCTCTCAGCCTTTTCTTATGGACCT | | |
| | TGAAGGAAAACAGGGTAATTTCAA | | |
| | AAATCTTAGGGAATTTGTGTTTAAG | | |
| | AATATTGATGGTTATTTTAAAATAT | | |
| | ATTCTAAGCACACGCCTATTAATTT | | |
| | AGTGCGTGATCTCCCTCAGGGTTTT | | |
| | TCGGCTTTAGAACCATTGGTAGATT | | |
| | TGCCAATAGGTATTAACATCACTA | | |
| | GGTTTCAAACTTTACTTGCTTTACA | | |
| | TAGAAGTTATTTGACTCCTGGTGAT | | |
| | TCTTCTTCAGGTTGGACAGCTGGTG | | |
| | CTGCAGCTTATTATGTGGGTTATCT | | |
| | TCAACCTAGGACTTTTCTATTAAAA | | |
| | TATAATGAAAATGGAACCATTACA | | |
| | GATGCTGTAGACTGTGCACTTGAC | | |
| | CCTCTCTCAGAAACAAAGTGTACG | | |
| | TTGAAATCC (SEQ ID NO: 2) | | |
| RBD- | AGAGTCCAACCAACAGAATCTATT | RVQPTESIVRFPNITNLCPFGEVF | Zhou et al., |
| SD1 | GTTAGATTTCCTAATATTACAAACT | NATRFASVYAWNRKRISNCVAD | Cell Rep. |
| (aa 319-591) | TGTGCCCTTTTGGTGAAGTTTTTAA | YSVLYNSASFSTFKCYGVSPTKL | 33: 108322 |
| | CGCCACCAGATTTGCATCTGTTTAT | NDLCFTNVYADSFVIRGDEVRQI | (2020) |
| | GCTTGGAACAGGAAGAGAATCAGC | APGQTGKIADYNYKLPDDFTGC | |
| | AACTGTGTTGCTGATTATTCTGTCC | VIAWNSNNLDSKVGGNYNYLYR | |
| | TATATAATTCCGCATCATTTTCCAC | LFRKSNLKPFERDISTEIYQAGST | |
| | TTTTAAGTGTTATGGAGTGTCTCCT | PCNGVEGFNCYFPLQSYGFQPTN | |
| | ACTAAATTAAATGATCTCTGCTTTA | GVGYQPYRVVVLSFELLHAPAT | |
| | CTAATGTCTATGCAGATTCATTTGT | VCGPKKSTNLVKNKCVNFNFNG | |
| | AATTAGAGGTGATGAAGTCAGACA | LTGTGVLTESNKKFLPFQQFGRD | |
| | AATCGCTCCAGGGCAAACTGGAAA | IADTTDAVRDPQTLEILDITPCS | |
| | GATTGCTGATTATAATTATAAATTA | (SEQ ID NO: 14) | |
| | CCAGATGATTTTACAGGCTGCGTTA | | |
| | TAGCTTGGAATTCTAACAATCTTGA | | |
| | TTCTAAGGTTGGTGGTAATTATAAT | | |
| | TACCTGTATAGATTGTTTAGGAAGT | | |
| | CTAATCTCAAACCTTTTGAGAGAG | | |
| | ATATTTCAACTGAAATCTATCAGGC | | |
| | CGGTAGCACACCTTGTAATGGTGTT | | |
| | GAAGGTTTTAATTGTTACTTTCCTT | | |
| | TACAATCATATGGTTTCCAACCCAC | | |
| | TAATGGTGTTGGTTACCAACCATAC | | |
| | AGAGTAGTAGTACTTTCTTTTGAAC | | |
| | TTCTACATGCACCAGCAACTGTTTG | | |
| | TGGACCTAAAAAGTCTACTAATTT | | |
| | GGTTAAAAACAAATGTGTCAATTT | | |
| | CAACTTCAATGGTTTAACAGGCAC | | |
| | AGGTGTTCTTACTGAGTCTAACAA | | |
| | AAAGTTTCTGCCTTTCCAACAATTT | | |
| | GGCAGAGACATTGCTGACACTACT | | |
| | GATGCTGTCCGTGATCCACAGACA | | |
| | CTTGAGATTCTTGACATTACACCAT | | |
| | GTTCT (SEQ ID NO: 3) | | |
| RBD | AGAGTCCAACCAACAGAATCTATT | RVQPTESIVRFPNITNLCPFGEVF | NCBI CDD: |
| (aa 319-541) | GTTAGATTTCCTAATATTACAAACT | NATRFASVYAWNRKRISNCVAD | 394827 |
| | TGTGCCCTTTTGGTGAAGTTTTTAA | YSVLYNSASFSTFKCYGVSPTKL | |
| | CGCCACCAGATTTGCATCTGTTTAT | NDLCFTNVYADSFVIRGDEVRQI | |
| | GCTTGGAACAGGAAGAGAATCAGC | APGQTGKIADYNYKLPDDFTGC | |
| | AACTGTGTTGCTGATTATTCTGTCC | VIAWNSNNLDSKVGGNYNYLYR | |
| | TATATAATTCCGCATCATTTTCCAC | LFRKSNLKPFERDISTEIYQAGST | |
| | TTTTAAGTGTTATGGAGTGTCTCCT | PCNGVEGFNCYFPLQSYGFQPTN | |
| | ACTAAATTAAATGATCTCTGCTTTA | GVGYQPYRVVVLSFELLHAPAT | |
| | CTAATGTCTATGCAGATTCATTTGT | VCGPKKSTNLVKNKCVNF | |
| | AATTAGAGGTGATGAAGTCAGACA | (SEQ ID NO: 15) | |
| | AATCGCTCCAGGGCAAACTGGAAA | | |
| | GATTGCTGATTATAATTATAAATTA | | |
| | CCAGATGATTTTACAGGCTGCGTTA | | |
| | TAGCTTGGAATTCTAACAATCTTGA | | |
| | TTCTAAGGTTGGTGGTAATTATAAT | | |
| | TACCTGTATAGATTGTTTAGGAAGT | | |
| | CTAATCTCAAACCTTTTGAGAGAG | | |
| | ATATTTCAACTGAAATCTATCAGGC | | |
| | CGGTAGCACACCTTGTAATGGTGTT | | |
| | GAAGGTTTTAATTGTTACTTTCCTT | | |
| | TACAATCATATGGTTTCCAACCCAC | | |
| | TAATGGTGTTGGTTACCAACCATAC | | |
| | AGAGTAGTAGTACTTTCTTTTGAAC | | |
| | TTCTACATGCACCAGCAACTGTTTG | | |
| | TGGACCTAAAAAGTCTACTAATTT | | |
| | GGTTAAAAACAAATGTGTCAATTT | | |
| | C (SEQ ID NO: 4) | | |
| RBD- | AGAGTCCAACCAACAGAATCTATT | RVQPTESIVRFPAITNLCPFGEVF | NCBI CDD: |
| C1N1 | GTTAGATTTCCTGCTATTACAAACT | NATRFASVYAWNRKRISNCVAD | 394827 |
| (aa 319-541) | TGTGCCCTTTTGGTGAAGTTTTTAA | YSVLYNSASFSTFKCYGVSPTKL | |
| | CGCCACCAGATTTGCATCTGTTTAT | NDLCFTNVYADSFVIRGDEVRQI | |
| | GCTTGGAACAGGAAGAGAATCAGC | APGQTGKIADYNYKLPDDFTGC | |
| | AACTGTGTTGCTGATTATTCTGTCC | VIAWNSNNLDSKVGGNYNYLYR | |
| | TATATAATTCCGCATCATTTTCCAC | LFRKSNLKPFERDISTEIYQAGST | |
| | TTTTAAGTGTTATGGAGTGTCTCCT | PCNGVEGFNCYFPLQSYGFQPTN | |
| | ACTAAATTAAATGATCTCTGCTTTA | GVGYQPYRVVVLSFELLHAPAT | |
| | CTAATGTCTATGCAGATTCATTTGT | VCGPKKSTNLVKNKAVNF | |
| | AATTAGAGGTGATGAAGTCAGACA | (SEQ ID NO: 16) | |
| | AATCGCTCCAGGGCAAACTGGAAA | | |
| | GATTGCTGATTATAATTATAAATTA | | |
| | CCAGATGATTTTACAGGCTGCGTTA | | |
| | TAGCTTGGAATTCTAACAATCTTGA | | |
| | TTCTAAGGTTGGTGGTAATTATAAT | | |
| | TACCTGTATAGATTGTTTAGGAAGT | | |
| | CTAATCTCAAACCTTTTGAGAGAG | | |
| | ATATTTCAACTGAAATCTATCAGGC | | |
| | CGGTAGCACACCTTGTAATGGTGTT | | |
| | GAAGGTTTTAATTGTTACTTTCCTT | | |
| | TACAATCATATGGTTTCCAACCCAC | | |
| | TAATGGTGTTGGTTACCAACCATAC | | |
| | AGAGTAGTAGTACTTTCTTTTGAAC | | |
| | TTCTACATGCACCAGCAACTGTTTG | | |
| | TGGACCTAAAAAGTCTACTAATTT | | |
| | GGTTAAAAACAAAGCTGTCAATTT | | |
| | C (SEQ ID NO: 5) | | |
| RBD (aa | TTTCCTAATATTACAAACTTGTGCC | FPNITNLCPFGEVFNATRFASVY | Zhou et al., |
| 329-526) | CTTTTGGTGAAGTTTTTAACGCCAC | AWNRKRISNCVADYSVLYNSAS | Cell Rep. |
| | CAGATTTGCATCTGTTTATGCTTGG | FSTFKCYGVSPTKLNDLCFTNVY | 33: 108322 |
| | AACAGGAAGAGAATCAGCAACTGT | ADSFVIRGDEVRQIAPGQTGKIA | (2020) |
| | GTTGCTGATTATTCTGTCCTATATA | DYNYKLPDDFTGCVIAWNSNNL | |
| | ATTCCGCATCATTTTCCACTTTTAA | DSKVGGNYNYLYRLFRKSNLKP | |
| | GTGTTATGGAGTGTCTCCTACTAAA | FERDISTEIYQAGSTPCNGVEGFN | |
| | TTAAATGATCTCTGCTTTACTAATG | CYFPLQSYGFQPTNGVGYQPYR | |
| | TCTATGCAGATTCATTTGTAATTAG | VVVLSFELLHAPATVCG | |
| | AGGTGATGAAGTCAGACAAATCGC | (SEQ ID NO: 17) | |
| | TCCAGGGCAAACTGGAAAGATTGC | | |
| | TGATTATAATTATAAATTACCAGAT | | |
| | GATTTTACAGGCTGCGTTATAGCTT | | |
| | GGAATTCTAACAATCTTGATTCTAA | | |
| | GGTTGGTGGTAATTATAATTACCTG | | |
| | TATAGATTGTTTAGGAAGTCTAATC | | |
| | TCAAACCTTTTGAGAGAGATATTTC | | |
| | AACTGAAATCTATCAGGCCGGTAG | | |
| | CACACCTTGTAATGGTGTTGAAGG | | |
| | TTTTAATTGTTACTTTCCTTTACAAT | | |
| | CATATGGTTTCCAACCCACTAATGG | | |
| | TGTTGGTTACCAACCATACAGAGT | | |
| | AGTAGTACTTTCTTTTGAACTTCTA | | |
| | CATGCACCAGCAACTGTTTGTGGA | | |
| | (SEQ ID NO: 6) | | |
| RBD-N1 | TTTCCTGCTATTACAAACTTGTGCC | FPAITNLCPFGEVFNATRFASVY | Zhou et al., |
| (aa 329-526) | CTTTTGGTGAAGTTTTTAACGCCAC | AWNRKRISNCVADYSVLYNSAS | Cell Rep. |
| | CAGATTTGCATCTGTTTATGCTTGG | FSTFKCYGVSPTKLNDLCFTNVY | 33: 108322 |
| | AACAGGAAGAGAATCAGCAACTGT | ADSFVIRGDEVRQIAPGQTGKIA | (2020) |
| | GTTGCTGATTATTCTGTCCTATATA | DYNYKLPDDFTGCVIAWNSNNL | |
| | ATTCCGCATCATTTTCCACTTTTAA | DSKVGGNYNYLYRLFRKSNLKP | |
| | GTGTTATGGAGTGTCTCCTACTAAA | FERDISTEIYQAGSTPCNGVEGFN | |
| | TTAAATGATCTCTGCTTTACTAATG | CYFPLQSYGFQPTNGVGYQPYR | |
| | TCTATGCAGATTCATTTGTAATTAG | VVVLSFELLHAPATVCG | |
| | AGGTGATGAAGTCAGACAAATCGC | (SEQ ID NO: 18) | |
| | TCCAGGGCAAACTGGAAAGATTGC | | |
| | TGATTATAATTATAAATTACCAGAT | | |
| | GATTTTACAGGCTGCGTTATAGCTT | | |
| | GGAATTCTAACAATCTTGATTCTAA | | |
| | GGTTGGTGGTAATTATAATTACCTG | | |
| | TATAGATTGTTTAGGAAGTCTAATC | | |
| | TCAAACCTTTTGAGAGAGATATTTC | | |
| | AACTGAAATCTATCAGGCCGGTAG | | |
| | CACACCTTGTAATGGTGTTGAAGG | | |
| | TTTTAATTGTTACTTTCCTTTACAAT | | |
| | CATATGGTTTCCAACCCACTAATGG | | |
| | TGTTGGTTACCAACCATACAGAGT | | |
| | AGTAGTACTTTCTTTTGAACTTCTA | | |
| | CATGCACCAGCAACTGTTTGTGGA | | |
| | (SEQ ID NO: 7) | | |
| RBD | TTTCCTAATATTACAAACTTGTGCC | FPNITNLCPFGEVFNATRFASVY | |
| (B.1.1.7 | CTTTTGGTGAAGTTTTTAACGCCAC | AWNRKRISNCVADYSVLYNSAS | |
| variant, | CAGATTTGCATCTGTTTATGCTTGG | FSTFKCYGVSPTKLNDLCFTNVY | |
| aa 329-526) | AACAGGAAGAGAATCAGCAACTGT | ADSFVIRGDEVRQIAPGQTGKIA | |
| | GTTGCTGATTATTCTGTCCTATATA | DYNYKLPDDFTGCVIAWNSNNL | |
| | ATTCCGCATCATTTTCCACTTTTAA | DSKVGGNYNYLYRLFRKSNLKP | |
| | GTGTTATGGAGTGTCTCCTACTAAA | FERDISTEIYQAGSTPCNGVEGFN | |
| | TTAAATGATCTCTGCTTTACTAATG | CYFPLQSYGFQPTYGVGYQPYR | |
| | TCTATGCAGATTCATTTGTAATTAG | VVVLSFELLHAPATVCG | |
| | AGGTGATGAAGTCAGACAAATCGC | (SEQ ID NO: 19) | |
| | TCCAGGGCAAACTGGAAAGATTGC | | |
| | TGATTATAATTATAAATTACCAGAT | | |
| | GATTTTACAGGCTGCGTTATAGCTT | | |
| | GGAATTCTAACAATCTTGATTCTAA | | |
| | GGTTGGTGGTAATTATAATTACCTG | | |
| | TATAGATTGTTTAGGAAGTCTAATC | | |
| | TCAAACCTTTTGAGAGAGATATTTC | | |
| | AACTGAAATCTATCAGGCCGGTAG | | |
| | CACACCTTGTAATGGTGTTGAAGG | | |
| | TTTTAATTGTTACTTTCCTTTACAAT | | |
| | CATATGGTTTCCAACCCACTTATGG | | |
| | TGTTGGTTACCAACCATACAGAGT | | |
| | AGTAGTACTTTCTTTTGAACTTCTA | | |
| | CATGCACCAGCAACTGTTTGTGGA | | |
| | (SEQ ID NO: 8) | | |
| RBD | TTTCCTAATATTACAAACTTGTGCC | FPNITNLCPFGEVFNATRFASVY | |
| (B.1.351 | CTTTTGGTGAAGTTTTTAACGCCAC | AWNRKRISNCVADYSVLYNSAS | |
| variant, | CAGATTTGCATCTGTTTATGCTTGG | FSTFKCYGVSPTKLNDLCFTNVY | |
| 329-526) | AACAGGAAGAGAATCAGCAACTGT | ADSFVIRGDEVRQIAPGQTGNIA | |
| | GTTGCTGATTATTCTGTCCTATATA | DYNYKLPDDFTGCVIAWNSNNL | |
| | ATTCCGCATCATTTTCCACTTTTAA | DSKVGGNYNYLYRLFRKSNLKP | |
| | GTGTTATGGAGTGTCTCCTACTAAA | FERDISTEIYQAGSTPCNGVKGF | |
| | TTAAATGATCTCTGCTTTACTAATG | NCYFPLQSYGFQPTYGVGYQPY | |
| | TCTATGCAGATTCATTTGTAATTAG | RVWLSFELLHAPATVCG | |
| | AGGTGATGAAGTCAGACAAATCGC | (SEQ ID NO: 20) | |
| | TCCAGGGCAAACTGGAAATATTGC | | |
| | TGATTATAATTATAAATTACCAGAT | | |
| | GATTTTACAGGCTGCGTTATAGCTT | | |
| | GGAATTCTAACAATCTTGATTCTAA | | |
| | GGTTGGTGGTAATTATAATTACCTG | | |
| | TATAGATTGTTTAGGAAGTCTAATC | | |
| | TCAAACCTTTTGAGAGAGATATTTC | | |
| | AACTGAAATCTATCAGGCCGGTAG | | |
| | CACACCTTGTAATGGTGTTAAAGG | | |
| | TTTTAATTGTTACTTTCCTTTACAAT | | |
| | CATATGGTTTCCAACCCACTTATGG | | |
| | TGTTGGTTACCAACCATACAGAGT | | |
| | AGTAGTACTTTCTTTTGAACTTCTA | | |
| | CATGCACCAGCAACTGTTTGTGGA | | |
| | (SEQ ID NO: 9) | | |
| RBD | TTTCCTAATATTACAAACTTGTGCC | FPNITNLCPFGEVFNATRFASVY | |
| (P.1 | CTTTTGGTGAAGTTTTTAACGCCAC | AWNRKRISNCVADYSVLYNSAS | |
| variant, | CAGATTTGCATCTGTTTATGCTTGG | FSTFKCYGVSPTKLNDLCFTNVY | |
| aa 329-526) | AACAGGAAGAGAATCAGCAACTGT | ADSFVIRGDEVRQIAPGQTGTIA | |
| | GTTGCTGATTATTCTGTCCTATATA | DYNYKLPDDFTGCVIAWNSNNL | |
| | ATTCCGCATCATTTTCCACTTTTAA | DSKVGGNYNYLYRLFRKSNLKP | |
| | GTGTTATGGAGTGTCTCCTACTAAA | FERDISTEIYQAGSTPCNGVKGF | |
| | TTAAATGATCTCTGCTTTACTAATG | NCYFPLQSYGFQPTYGVGYQPY | |
| | TCTATGCAGATTCATTTGTAATTAG | RVWLSFELLHAPATVCG | |
| | AGGTGATGAAGTCAGACAAATCGC | (SEQ ID NO: 21) | |
| | TCCAGGGCAAACTGGAACGATTGC | | |
| | TGATTATAATTATAAATTACCAGAT | | |
| | GATTTTACAGGCTGCGTTATAGCTT | | |
| | GGAATTCTAACAATCTTGATTCTAA | | |
| | GGTTGGTGGTAATTATAATTACCTG | | |
| | TATAGATTGTTTAGGAAGTCTAATC | | |
| | TCAAACCTTTTGAGAGAGATATTTC | | |
| | AACTGAAATCTATCAGGCCGGTAG | | |
| | CACACCTTGTAATGGTGTTAAAGG | | |
| | TTTTAATTGTTACTTTCCTTTACAAT | | |
| | CATATGGTTTCCAACCCACTTATGG | | |
| | TGTTGGTTACCAACCATACAGAGT | | |
| | AGTAGTACTTTCTTTTGAACTTCTA | | |
| | CATGCACCAGCAACTGTTTGTGGA | | |
| | (SEQ ID NO: 10) | | |
| RBD | TTTCCTAATATTACAAACTTGTGCC | FPNITNLCPFGEVFNATRFASVY | |
| (B.1.617. | CTTTTGGTGAAGTTTTTAACGCCAC | AWNRKRISNCVADYSVLYNSAS | |
| 2 variant, | CAGATTTGCATCTGTTTATGCTTGG | FSTFKCYGVSPTKLNDLCFTNVY | |
| aa 329-526) | AACAGGAAGAGAATCAGCAACTGT | ADSFVIRGDEVRQIAPGQTGTIA | |
| | GTTGCTGATTATTCTGTCCTATATA | DYNYKLPDDFTGCVIAWNSNNL | |
| | ATTCCGCATCATTTTCCACTTTTAA | DSKVGGNYNYLYRLFRKSNLKP | |
| | GTGTTATGGAGTGTCTCCTACTAAA | FERDISTEIYQAGSTPCNGVKGF | |
| | TTAAATGATCTCTGCTTTACTAATG | NCYFPLQSYGFQPTYGVGYQPY | |
| | TCTATGCAGATTCATTTGTAATTAG | RVVVLSFELLHAPATVCG | |
| | AGGTGATGAAGTCAGACAAATCGC | (SEQ ID NO: 22) | |
| | TCCAGGGCAAACTGGAACGATTGC | | |
| | TGATTATAATTATAAATTACCAGAT | | |
| | GATTTTACAGGCTGCGTTATAGCTT | | |
| | GGAATTCTAACAATCTTGATTCTAA | | |
| | GGTTGGTGGTAATTATAATTACCTG | | |
| | TATAGATTGTTTAGGAAGTCTAATC | | |
| | TCAAACCTTTTGAGAGAGATATTTC | | |
| | AACTGAAATCTATCAGGCCGGTAG | | |
| | CACACCTTGTAATGGTGTTAAAGG | | |
| | TTTTAATTGTTACTTTCCTTTACAAT | | |
| | CATATGGTTTCCAACCCACTTATGG | | |
| | TGTTGGTTACCAACCATACAGAGT | | |
| | AGTAGTACTTTCTTTTGAACTTCTA | | |
| | CATGCACCAGCAACTGTTTGTGGA | | |
| | (SEQ ID NO: 11) | | |

In certain embodiments, a genetically-engineered cell of the present disclosure can express and secrete an antigen disclosed herein and can further express a cell targeting molecule. In certain embodiments, the cell targeting molecule can be secreted from the genetically-engineered cell. In certain embodiments, a genetically-engineered cell disclosed herein can secrete an antigen and secrete a cell targeting molecule. In certain embodiments, the cell targeting molecule can be displayed on the cell surface of the genetically-engineered cell. In certain embodiments, a genetically-engineered cell disclosed herein can secrete an antigen and display a cell targeting molecule on its surface.

Alternatively or additionally, the antigen can be a fusion protein that comprises the peptide or a protein or fragment thereof derived from a coronavirus and further comprises a cell targeting molecule. In certain embodiments, the antigen is a fusion protein that comprises an S protein or a fragment thereof. In certain embodiments, the antigen is a fusion protein that comprises a fragment of an S protein, e.g., an RBD of an S protein. For example, but not by way of limitation, the antigen is a fusion protein that comprises an amino acid sequence of any one of SEQ ID NOs: 12-22 or an amino acid sequence encoded by a nucleotide sequence of any one of SEQ ID NOs: 1-11.

In certain embodiments, the cell targeting molecule can be a targeting molecule for targeting an immune cell, e.g., an immune cell targeting molecule. In certain embodiments, the cell targeting molecule can facilitate the attachment of the antigen to immune cells, e.g., antigen-presenting cells such as dendritic cells. For example, but not by way of limitation, an immune cell targeting molecule can be a protein or fragment thereof that targets the antigen and/or the genetically-engineered cell secreting the antigen to mucosal immune tissues such as Peyer's patches in the small intestines. Non-limiting examples of cell targeting molecules include C-terminal Clostridium perfringens Enterotoxin (C-CPE), a Cholera Toxin Subunit B (CTB), a heat-labile enterotoxin B subunit (LTB), a C-terminal Clostridium difficile toxin A (TxA(C314)), CRM197 (a diphtheria toxin mutant), fragment C of tetanus toxoid, a cholesteryl group-bearing pullulan (CHP), PT-9K/129G (a detoxified derivative of pertussis toxin) or mutants thereof. In certain embodiments, the cell targeting molecule is a cholera toxin or a fragment thereof, e.g., a CTB. In certain embodiments, the cell targeting molecule is an LTB. In certain embodiments, the cell targeting molecule is a C-CPE. Non-limiting examples of amino acid sequences (and corresponding nucleotide sequence) for use as cell targeting molecules are disclosed in Table 3.

In certain embodiments, a CTB for use as cell targeting molecule comprises the amino acid sequence of GenBank Accession No. U25679. In certain embodiments, a CTB for use as cell targeting molecule is encoded by a nucleotide sequence comprising the nucleotide sequence of GenBank Accession No. U25679.

In certain embodiments, a CTB for use as cell targeting molecule comprises or consists of an amino acid sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the amino acid sequence set forth in SEQ ID NO: 26. In certain embodiments, a CTB for use as cell targeting molecule comprises or consists of the amino acid sequence of SEQ ID NO: 26. In certain embodiments, a CTB for use as cell targeting molecule can be encoded by a nucleotide sequence that comprises or consists of a nucleotide sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the nucleotide sequence set forth in SEQ ID NO: 25. In certain embodiments, a CTB for use as cell targeting molecule can be encoded by a nucleotide sequence that comprises or consists of the nucleotide sequence of SEQ ID NO: 25.

In certain embodiments, an LTB for use as cell targeting molecule comprises the amino acid sequence of GenBank Accession No. M17873. In certain embodiments, an LTB for use as cell targeting molecule is encoded by a nucleotide sequence comprising the nucleotide sequence of GenBank Accession No. M17873.

In certain embodiments, an LTB for use as cell targeting molecule comprises or consists of an amino acid sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the amino acid sequence set forth in SEQ ID NO: 28. In certain embodiments, an LTB for use as cell targeting molecule comprises or consists of the amino acid sequence of SEQ ID NO: 28. In certain embodiments, an LTB for use as cell targeting molecule can be encoded by a nucleotide sequence that comprises or consists of a nucleotide sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the nucleotide sequence set forth in SEQ ID NO: 27. In certain embodiments, an LTB for use as cell targeting molecule can be encoded by a nucleotide sequence that comprises or consists of the nucleotide sequence of SEQ ID NO: 27.

In certain embodiments, a C-CPE for use as cell targeting molecule comprises the amino acid sequence of GenBank Accession No. M98037. In certain embodiments, a C-CPE for use as cell targeting molecule is encoded by a nucleotide sequence comprising the nucleotide sequence of GenBank Accession No. M98037.

In certain embodiments, a C-CPE for use as cell targeting molecule comprises or consists of an amino acid sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the amino acid sequence set forth in SEQ ID NO: 24. In certain embodiments, a C-CPE for use as cell targeting molecule comprises or consists of the amino acid sequence of SEQ ID NO: 24. In certain embodiments, a C-CPE for use as cell targeting molecule can be encoded by a nucleotide sequence that comprises or consists of a nucleotide sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the nucleotide sequence set forth in SEQ ID NO: 23. In certain embodiments, a C-CPE for use as cell targeting molecule can be encoded by a nucleotide sequence that comprises or consists of the nucleotide sequence of SEQ ID NO: 23.

In certain embodiments, a fragment C of tetanus toxoid for use as cell targeting molecule comprises or consists of an amino acid sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the amino acid sequence set forth in SEQ ID NO: 37. In certain embodiments, a fragment C of tetanus toxoid for use as cell targeting molecule comprises or consists of the amino acid sequence of SEQ ID NO: 37. In certain embodiments, a fragment C of tetanus toxoid for use as cell targeting molecule can be encoded by a nucleotide sequence that comprises or consists of a nucleotide sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the nucleotide sequence set forth in SEQ ID NO: 36. In certain embodiments, a fragment C of tetanus toxoid for use as cell targeting molecule can be encoded by a nucleotide sequence that comprises or consists of the nucleotide sequence of SEQ ID NO:

36.

In certain embodiments, PT-9K/129G for use as cell targeting molecule comprises or consists of an amino acid sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the amino acid sequence set forth in SEQ ID NO: 39. In certain embodiments, PT-9K/129G for use as cell targeting molecule comprises or consists of the amino acid sequence of SEQ ID NO: 39. In certain embodiments, PT-9K/129G for use as cell targeting molecule can be encoded by a nucleotide sequence that comprises or consists of a nucleotide sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the nucleotide sequence set forth in SEQ ID NO: 38. In certain embodiments, PT-9K/129G for use as cell targeting molecule can be encoded by a nucleotide sequence that comprises or consists of the nucleotide sequence of SEQ ID NO: 38.

In certain embodiments, a C-terminal Clostridium difficile toxin A (TxA(C314)) for use as cell targeting molecule comprises the amino acid sequence of GenBank Accession No. M30307.1. In certain embodiments, C-terminal Clostridium difficile toxin A (TxA(C314)) for use as cell targeting molecule is encoded by a nucleotide sequence comprising the nucleotide sequence of GenBank Accession No. M30307.1.

In certain embodiments, a C-terminal Clostridium difficile toxin A (TxA(C314)) for use as cell targeting molecule comprises or consists of an amino acid sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the amino acid sequence set forth in SEQ ID NO: 41. In certain embodiments, a C-terminal Clostridium difficile toxin A (TxA(C314)) for use as cell targeting molecule comprises or consists of the amino acid sequence of SEQ ID NO: 41. In certain embodiments, a C-terminal Clostridium difficile toxin A (TxA(C314)) for use as cell targeting molecule can be encoded by a nucleotide sequence that comprises or consists of a nucleotide sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the nucleotide sequence set forth in SEQ ID NO: 40. In certain embodiments, a C-terminal Clostridium difficile toxin A (TxA(C314)) for use as cell targeting molecule can be encoded by a nucleotide sequence that comprises or consists of the nucleotide sequence of SEQ ID NO: 40.

In certain embodiments, CRM-197 for use as cell targeting molecule comprises the amino acid sequence of GenBank Accession No. KU521393.1. In certain embodiments, CRM-197 for use as cell targeting molecule is encoded by a nucleotide sequence comprising the nucleotide sequence of GenBank Accession No. KU521393.1.

In certain embodiments, CRM-197 for use as cell targeting molecule comprises or consists of an amino acid sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the amino acid sequence set forth in SEQ ID NO: 43. In certain embodiments, CRM-197 for use as cell targeting molecule comprises or consists of the amino acid sequence of SEQ ID NO: 43. In certain embodiments, CRM-197 for use as cell targeting molecule can be encoded by a nucleotide sequence that comprises or consists of a nucleotide sequence that is at least about 80%, at least about 85%, at least about 90%, at least about 91%, at least about 92%, at least about 93%, at least about 94%, at least about 95%, at least about 96%, at least about 97%, at least about 98% or at least about 99% homologous to the nucleotide sequence set forth in SEQ ID NO: 42. In certain embodiments, CRM-197 for use as cell targeting molecule can be encoded by a nucleotide sequence that comprises or consists of the nucleotide sequence of SEQ ID NO: 42.

TABLE 3

| Cell Targeting | | | |
|---|---|---|---|
| Molecule | Nucleotide Sequence | Amino Acid Sequence | Source |
| CTB | ATGATTAAATTAAAATTTGGTG | MIKLKFGVFFTVLLSSAYAHGTP | GenBank |
| | TTTTTTTTACAGTTTTACTATCT | QNITDLCAEYHNTQIHTLNDKIFS | Accession |
| | TCAGCATATGCACATGGAACAC | YTESLAGKREMAIITFKNGATFQ | No. U25679 |
| | CTCAAAATATTACTGATTTGTG | VEVPGSQHIDSQKKAIERMKDTL | |
| | TGCAGAATACCACAACACACA | RIAYLTEAKVEKLCVWNNKTPH | |
| | AATACATACGCTAAATGATAA | AIAAISMAN (SEQ ID NO: 26) | |
| | GATATTTTCGTATACAGAATCT | | |
| | CTAGCTGGAAAAAGAGAGATG | | |
| | GCTATCATTACTTTTAAGAATG | | |
| | GTGCAACTTTTCAAGTAGAAGT | | |
| | ACCAGGTAGTCAACATATAGAT | | |
| | TCACAAAAAAAAGCGATTGAA | | |
| | AGGATGAAGGATACCCTGAGG | | |
| | ATTGCATATCTTACTGAAGCTA | | |
| | AAGTCGAAAAGTTATGTGTATG | | |
| | GAATAATAAAACGCCTCATGC | | |
| | GATTGCCGCAATTAGTATGGCA | | |
| | AATTAA (SEQ ID NO: 25) | | |
| LTB | ATGAATAAAGTAAAATGTTATG | MNKVKCYVLFTALLSSLYAHGA | GenBank |
| | TTTTATTTACGGCGTTACTATCC | PQTITELCSEYRNTQIYTINDKILS | Accession |
| | TCTCTATATGCACACGGAGCTC | YTESMAGKREMVIITFKSGETFQ | No. M17873 |
| | CCCAGACTATTACAGAACTATG | VEVPGSQHIDSQKKAIERMKDTL | |
| | TTCGGAATATCGCAACACACAA | RITYLTETKIDKLCVWNNKTPNS | |
| | ATATATACGATAAATGACAAG | IAAISMKN (SEQ ID NO: 28) | |
| | ATACTATCATATACGGAATCGA | | |
| | TGGCAGGCAAAAGAGAAATGG | | |
| | TTATCATTACATTTAAGAGCGG | | |
| | CGAAACATTTCAGGTCGAAGTC | | |
| | CCGGGCAGTCAACATATAGACT | | |
| | CCCAGAAAAAAGCCATTGAAA | | |
| | GGATGAAGGACACATTAAGAA | | |
| | TCACATATCTGACCGAGACCAA | | |
| | AATTGATAAATTATGTGTATGG | | |
| | AATAATAAAACCCCCAATTCAA | | |
| | TTGCGGCAATCAGTATGAAAA | | |
| | AC (SEQ ID NO: 27) | | |
| C-CPE | GATATAGAAAAAGAAATCCTT | DIEKEILDLAAATERLNLTDALN | GenBank |
| (aa 194-319 of the | GATTTAGCTGCTGCTACAGAAA | SNPAGNLYDWRSSNSYPWTQKL | Accession |
| Clostridium | GATTAAATTTAACTGATGCATT | NLHLTITATGQKYRILASKIVDFN | No. M98037 |
| perfringens | AAACTCAAATCCAGCTGGTAAT | IYSNNFNNLVKLEQSLGDGVKD | |
| enterotoxin) | TTATATGATTGGCGTTCTTCTA | HYVDISLDAGQYVLVMKANSSY | |
| | ACTCATACCCTTGGACTCAAAA | SGNYPYSILFQKF | |
| | GCTTAATTTACACTTAACAATT | (SEQ ID NO: 24) | |
| | ACAGCTACTGGACAAAAATAT | | |
| | AGAATCTTAGCTAGCAAAATTG | | |
| | TTGATTTTAATATTTATTCAAAT | | |
| | AATTTTAATAATCTAGTGAAAT | | |
| | TAGAACAGTCCTTAGGTGATGG | | |
| | AGTAAAAGATCATTATGTTGAT | | |
| | ATAAGCTTAGATGCTGGACAAT | | |
| | ATGTTCTTGTAATGAAAGCTAA | | |
| | TTCATCATATAGTGGAAATTAC | | |
| | CCTTATTCAATATTATTTCAAA | | |
| | AATTT (SEQ ID NO: 23) | | |
| Fragment Cof | ATGGGCAGCAGCCATCATCATC | MGSSHHHHHHSSGLVPRGSHMK | |
| tetanus toxoid | ATCATCACAGCAGCGGCCTGGT | NLDCWVDNEEDIDVILKKSTILN | |
| | GCCGCGCGGCAGCCATATGAA | LDINNDIISDISGFNSSVITYPDAQ | |
| | AAACCTTGATTGTTGGGTCGAC | LVPGINGKAIHLVNNESSEVIVH | |
| | AACGAAGAAGACATCGATGTT | KAMDIEYNDMFNNFTVSFWLRV | |
| | ATCCTGAAAAAGTCTACCATTC | PKVSASHLEQYGTNEYSIISSMK | |
| | TGAACTTGGACATCAACAACG | KHSLSIGSGWSVSLKGNNLIWTL | |
| | ATATTATCTCCGACATCTCTGG | KDSAGEVRQITFRDLPDKFNAYL | |
| | TTTCAACTCCTCTGTTATCACAT | ANKWVFITITNDRLSSANLYING | |
| | ATCCAGATGCTCAATTGGTGCC | VLMGSAEITGLGAIREDNNITLK | |
| | GGGCATCAACGGCAAAGCTAT | LDRCNNNNQYVSIDKFRIFCKAL | |
| | CCACCTGGTTAACAACGAATCT | NPKEIEKLYTSYLSITFLRDFWGN | |
| | TCTGAAGTTATCGTGCACAAGG | PLRYDTEYYLIPVASSSKDVQLK | |
| | CCATGGACATCGAATACAACG | NITDYMYLTNAPSYTNGKLNIYY | |
| | ACATGTTCAACAACTTCACCGT | RRLYNGLKFIIKRYTPNNEIDSFV | |
| | TAGCTTCTGGCTGCGCGTTCCG | KSGDFIKLYVSYNNNEHIVGYPK | |
| | AAAGTTTCTGCTTCCCACCTGG | DGNAFNNLDRILRVGYNAPGIPL | |
| | AACAGTACGGCACTAACGAGT | YKKMEAVKLRDLKTYSVQLKL | |
| | ACTCCATCATCAGCTCTATGAA | YDDKNASLGLVGTHNGQIGNDP | |
| | GAAACACTCCCTGTCCATCGGC | NRDILIASNWYFNHLKDKILGCD | |
| | TCTGGTTGGTCTGTTTCCCTGA | WYFVPTDEGWTND | |
| | AGGGTAACAACCTGATCTGGA | (SEQ ID NO: 37) | |
| | CTCTGAAAGACTCCGCGGGCG | | |
| | AAGTTCGTCAGATCACTTTCCG | | |
| | CGACCTGCCGGACAAGTTCAAC | | |
| | GCGTACCTGGCTAACAAATGG | | |
| | GTTTTCATCACTATCACTAACG | | |
| | ATCGTCTGTCTTCTGCTAACCT | | |
| | GTACATCAACGGCGTTCTGATG | | |
| | GGCTCCGCTGAAATCACTGGTC | | |
| | TGGGCGCTATCCGTGAGGACA | | |
| | ACAACATCACTCTTAAGCTGGA | | |
| | CCGTTGCAACAACAACAACCA | | |
| | GTACGTATCCATCGACAAGTTC | | |
| | CGTATCTTCTGCAAAGCACTGA | | |
| | ACCCGAAAGAGATCGAAAAAC | | |
| | TGTATACCAGCTACCTGTCTAT | | |
| | CACCTTCCTGCGTGACTTCTGG | | |
| | GGTAACCCGCTGCGTTACGACA | | |
| | CCGAATATTACCTGATCCCGGT | | |
| | AGCTTCTAGCTCTAAAGACGTT | | |
| | CAGCTGAAAAACATCACTGACT | | |
| | ACATGTACCTGACCAACGCGCC | | |
| | GTCCTACACTAACGGTAAACTG | | |
| | AACATCTACTACCGACGTCTGT | | |
| | ACAACGGCCTGAAATTCATCAT | | |
| | CAAACGCTACACTCCGAACAA | | |
| | CGAAATCGATTCTTTCGTTAAA | | |
| | TCTGGTGACTTCATCAAACTGT | | |
| | ACGTTTCTTACAACAACAACGA | | |
| | ACACATCGTTGGTTACCCGAAA | | |
| | GACGGTAACGCTTTCAACAACC | | |
| | TGGACAGAATTCTGCGTGTTGG | | |
| | TTACAACGCTCCGGGTATCCCG | | |
| | CTGTACAAAAAAATGGAAGCT | | |
| | GTTAAACTGCGTGACCTGAAAA | | |
| | CCTACTCTGTTCAGCTGAAACT | | |
| | GTACGACGACAAAAACGCTTCT | | |
| | CTGGGTCTGGTTGGTACCCACA | | |
| | ACGGTCAGATCGGTAACGACC | | |
| | CGAACCGTGACATCCTGATCGC | | |
| | TTCTAACTGGTACTTCAACCAC | | |
| | CTGAAAGACAAAATCCTGGGTT | | |
| | GCGACTGGTACTTCGTTCCGAC | | |
| | CGATGAAGGTTGGACCAACGA | | |
| | CTAA (SEQ ID NO: 36) | | |
| Mutant (PTX- | ATGCGTTGCACTCGGGCAATTC | MRCTRAIRQTARTGWLTWLAIL | |
| 9K/129G) | GCCAAACCGCAAGAACAGGCT | AVTAPVTSPAWADDPPATVYKY | |
| Subunit S1 of | GGCTGACGTGGCTGGCGATTCT | DSRPPEDVFQNGFTAWGNNDNV | |
| pertussis toxin | TGCCGTCACGGCGCCCGTGACT | LDHLTGRSCQVGSSNSAFVSTSS | |
| | TCGCCGGCATGGGCCGACGATC | SRRYTEVYLEHRMQEAVEAERA | |
| | CTCCCGCCACCGTATACAAATA | GRGTGHFIGYIYEVRADNNFYG | |
| | TGACTCCCGCCCGCCGGAGGAC | AASSYFEYVDTYGDNAGRILAG | |
| | GTTTTCCAGAACGGATTCACGG | ALATYQSGYLAHRRIPPENIRRV | |
| | CGTGGGGAAACAACGACAATG | TRVYHNGITGETTTTEYSNARYV | |
| | TGCTCGACCATCTGACCGGACG | SQQTRANPNPYTSRRSVASIVGT | |
| | TTCCTGCCAGGTCGGCAGCAGC | LVRMAPVIGACMARQAESSEAM | |
| | AACAGCGCTTTCGTCTCCACCA | AAWSERAGEAMVLVYYESIAYS | |
| | GCAGCAGCCGGCGCTATACCG | F (SEQ ID NO: 39) | |
| | AGGTCTATCTCGAACATCGCAT | | |
| | GCAGGAAGCGGTCGAGGCCGA | | |
| | ACGCGCCGGCAGGGGCACCGG | | |
| | CCACTTCATCGGCTACATCTAC | | |
| | GAAGTCCGCGCCGACAACAAT | | |
| | TTCTACGGCGCCGCCAGCTCGT | | |
| | ACTTCGAATACGTCGACACTTA | | |
| | TGGCGACAATGCCGGCCGTATC | | |
| | CTCGCCGGCGCGCTGGCCACCT | | |
| | ACCAGAGCGGCTATCTGGCAC | | |
| | ACCGGCGCATTCCGCCCGAAA | | |
| | ACATCCGCAGGGTAACGCGGG | | |
| | TCTATCACAACGGCATCACCGG | | |
| | CGAGACCACGACCACGGAGTA | | |
| | TTCCAACGCTCGCTACGTCAGC | | |
| | CAGCAGACTCGCGCCAATCCCA | | |
| | ACCCCTACACATCGCGAAGGTC | | |
| | CGTAGCGTCGATCGTCGGCACA | | |
| | TTGGTGCGCATGGCGCCGGTGA | | |
| | TAGGCGCTTGCATGGCGCGGCA | | |
| | GGCCGAAAGCTCCGAGGCCAT | | |
| | GGCAGCCTGGTCCGAACGCGC | | |
| | CGGCGAGGCGATGGTTCTCGTG | | |
| | TACTACGAAAGCATCGCGTATT | | |
| | CGTTCTAG (SEQ ID NO: 38) | | |
| C-terminal | ATTAATGGTAAACATTTTTATT | INGKHFYFNTDGIMQIGVFKGPN | GenBank |
| Clostridium | TTAATACTGATGGTATTATGCA | GFEYFAPANTDANNIEGQAILYQ | Accession |
| difficile toxin A | GATAGGAGTGTTTAAAGGACCT | NKFLTLNGKKYYFGSDSKAVTG | No. |
| (TxA(C314)) | AATGGATTTGAATACTTTGCAC | LRTIDGKKYYFNTNTAVAVTGW | M30307.1 |
| | CTGCTAATACGGATGCTAACAA | QTINGKKYYFNTNTSIASTGYTII | |
| | CATAGAAGGTCAAGCTATACTT | SGKHFYFNTDGIMQIGVFKGPDG | |
| | TACCAAAATAAATTCTTAACTT | FEYFAPANTDANNIEGQAIRYQN | |
| | TGAATGGTAAAAAATATTACTT | RFLYLHDNIYYFGNNSKAATGW | |
| | TGGTAGTGACTCAAAAGCAGTT | VTIDGNRYYFEPNTAMGANGYK | |
| | ACCGGACTGCGAACTATTGATG | TIDNKNFYFRNGLPQIGVFKGSN | |
| | GTAAAAAATATTACTTTAATAC | GFEYFAPANTDANNIEGQAIRYQ | |
| | TAACACTGCTGTTGCAGTTACT | NRFLHLLGKIYYFGNNSKAVTG | |
| | GGATGGCAAACTATTAATGGTA | WQTINGKVYYFMPDTAMAAAG | |
| | AAAAATACTACTTTAATACTAA | GLFEIDGVIYFFGVDGVKAP | |
| | CACTTCTATAGCTTCAACTGGT | (SEQ ID NO: 41) | |
| | TATACAATTATTAGTGGTAAAC | | |
| | ATTTTTATTTTAATACTGATGGT | | |
| | ATTATGCAGATAGGAGTGTTTA | | |
| | AAGGACCTGATGGATTTGAATA | | |
| | CTTTGCACCTGCTAATACAGAT | | |
| | GCTAACAATATAGAAGGTCAA | | |
| | GCTATACGTTATCAAAATAGAT | | |
| | TCCTATATTTACATGACAATAT | | |
| | ATATTATTTTGGTAATAATTCA | | |
| | AAAGCGGCTACTGGTTGGGTA | | |
| | ACTATTGATGGTAATAGATATT | | |
| | ACTTCGAGCCTAATACAGCTAT | | |
| | GGGTGCGAATGGTTATAAAACT | | |
| | ATTGATAATAAAAATTTTTACT | | |
| | TTAGAAATGGTTTACCTCAGAT | | |
| | AGGAGTGTTTAAAGGGTCTAAT | | |
| | GGATTTGAATACTTTGCACCTG | | |
| | CTAATACGGATGCTAACAATAT | | |
| | AGAAGGTCAAGCTATACGTTAT | | |
| | CAAAATAGATTCCTACATTTAC | | |
| | TTGGAAAAATATATTACTTTGG | | |
| | TAATAATTCAAAAGCAGTTACT | | |
| | GGATGGCAAACTATTAATGGTA | | |
| | AAGTATATTACTTTATGCCTGA | | |
| | TACTGCTATGGCTGCAGCTGGT | | |
| | GGACTTTTCGAGATTGATGGTG | | |
| | TTATATATTTCTTTGGTGTTGAT | | |
| | GGAGTAAAAGCCCCT | | |
| | (SEQ ID NO: 40) | | |
| CRM-197 | ATGGGCGCAGACGATGTTGTG | MGADDVVDSSKSFVMENFSSYH | GenBank |
| | GACTCAAGTAAATCATTTGTCA | GTKPGYVDSIQKGIQKPKSGTQG | Accession |
| | TGGAAAACTTCTCCTCATATCA | NYDDDWKEFYSTDNKYDAAGY | No. |
| | CGGCACGAAACCGGGCTACGT | SVDNENPLSGKAGGVVKVTYPG | KU521393.1 |
| | TGATAGCATTCAGAAAGGTATC | LTKVLALKVDNAETIKKELGLSL | |
| | CAAAAACCGAAATCTGGCACG | TEPLMEQVGTEEFIKRFGDGASR | |
| | CAGGGTAACTACGATGACGATT | VVLSLPFAEGSSSVEYINNWEQA | |
| | GGAAAGAATTCTACAGCACCG | KALSVELEINFETRGKRGQDAM | |
| | ACAACAAATATGATGCGGCCG | YEYMAQACAGNRVRRSVGSSLS | |
| | GTTACTCAGTCGACAACGAAA | CINLDWDVIRDKTKTKIESLKEH | |
| | ATCCGCTGTCGGGCAAAGCCG | GPIKNKMSESPNKTVSEEKAKQY | |
| | GCGGTGTGGTTAAAGTGACGTA | LEEFHQTALEHPELSELKTVTGT | |
| | TCCGGGCCTGACCAAAGTCCTG | NPVFAGANYAAWAVNVAQVID | |
| | GCCCTGAAAGTGGATAATGCA | SETADNLEKTTAALSILPGIGSV | |
| | GAAACCATCAAAAAAGAACTG | MGIADGAVHHNTEEIVAQSIALS | |
| | GGTCTGAGCCTGACGGAACCG | SLMVAQAIPLVGELVDIGFAAYN | |
| | CTGATGGAACAGGTTGGCACC | FVESIINLFQVVHNSYNRPAYSPG | |
| | GAAGAATTTATCAAACGCTTCG | HKTQPFLHDGYAVSWNTVEDSII | |
| | GCGATGGTGCCAGTCGTGTCGT | RTGFQGESGHDIKITAENTPLPIA | |
| | GCTGTCCCTGCCGTTCGCAGAA | GVLLPTIPGKLDVNKSKTHISVN | |
| | GGTAGCTCTAGTGTGGAATATA | GRKIRMRCRAIDGDVTFCRPKSP | |
| | TTAACAATTGGGAACAAGCGA | VYVGNGVHANLHVAFHRSSSEK | |
| | AAGCCCTGTCCGTTGAACTGGA | IHSNEISSDSIGVLGYQKTVDHTK | |
| | AATCAACTTTGAAACCCGCGGC | VNSKLSLFFEIKS | |
| | AAACGTGGTCAGGATGCGATG | (SEQ ID NO: 43) | |
| | TATGAATACATGGCACAAGCTT | | |
| | GCGCGGGTAATCGCGTTCGTCG | | |
| | CAGCGTCGGCTCCTCACTGTCT | | |
| | TGTATCAACCTGGACTGGGATG | | |
| | TTATCCGTGATAAAACCAAAAC | | |
| | GAAAATCGAAAGTCTGAAAGA | | |
| | ACATGGCCCGATCAAAAACAA | | |
| | AATGAGCGAATCTCCGAATAA | | |
| | AACGGTGTCCGAAGAAAAAGC | | |
| | TAAACAGTATCTGGAAGAATTC | | |
| | CACCAAACCGCACTGGAACAT | | |
| | CCGGAACTGTCAGAACTGAAA | | |
| | ACCGTGACGGGTACCAACCCG | | |
| | GTTTTTGCCGGCGCAAATTACG | | |
| | CAGCTTGGGCTGTGAACGTTGC | | |
| | GCAAGTGATTGACTCGGAAAC | | |
| | GGCCGATAATCTGGAAAAAAC | | |
| | CACGGCGGCCCTGAGTATTCTG | | |
| | CCGGGCATCGGTTCCGTTATGG | | |
| | GTATTGCCGACGGCGCAGTCCA | | |
| | TCACAACACCGAAGAAATTGT | | |
| | GGCCCAGTCTATCGCACTGTCG | | |
| | AGCCTGATGGTTGCTCAAGCGA | | |
| | TTCCGCTGGTTGGCGAACTGGT | | |
| | TGATATCGGCTTTGCAGCTTAC | | |
| | AACTTCGTGGAAAGTATTATCA | | |
| | ACCTGTTTCAGGTTGTCCACAA | | |
| | CTCATATAATCGCCCGGCCTAC | | |
| | TCGCCGGGTCACAAAACCCAA | | |
| | CCGTTCCTGCATGACGGCTACG | | |
| | CGGTTAGCTGGAATACGGTCGA | | |
| | AGATTCTATTATCCGTACCGGC | | |
| | TTTCAGGGTGAATCTGGCCACG | | |
| | ACATTAAAATCACGGCTGAAA | | |
| | ACACCCCGCTGCCGATTGCAGG | | |
| | TGTTCTGCTGCCGACGATCCCG | | |
| | GGTAAACTGGATGTTAACAAAT | | |
| | CAAAAACCCATATCTCGGTCAA | | |
| | CGGTCGCAAAATTCGTATGCGC | | |
| | TGCCGTGCGATCGACGGCGATG | | |
| | TGACCTTCTGTCGTCCGAAAAG | | |
| | CCCGGTCTATGTGGGCAACGGT | | |
| | GTCCATGCTAATCTGCACGTGG | | |
| | CGTTTCATCGCTCTAGTTCCGA | | |
| | AAAAATCCATAGTAACGAAAT | | |
| | CTCATCGGATTCCATTGGTGTG | | |
| | CTGGGCTACCAGAAAACCGTG | | |
| | GACCATACCAAAGTGAATAGC | | |
| | AAACTGAGCCTGTTCTTCGAAA | | |
| | TCAAATCGTAA (SEQ ID NO: 42) | | |

In certain embodiments, the antigen is a fusion protein that comprises an S protein or fragment thereof, e.g., an RBD of an S protein, and a cell targeting molecule. For example, but not by way of limitation, the antigen is a fusion protein comprising an RBD of an S protein from SARS-CoV-2 and a cell targeting molecule. In certain embodiments, the antigen is a fusion protein comprising an RBD of an S protein from SARS-CoV-2 and an CTB. In certain embodiments, the antigen is a fusion protein comprising the amino acid sequence set forth in SEQ ID NO: 15, 16, 17 or 18 and the amino acid sequence set forth in SEQ ID NO: 26.

In certain embodiments, the peptide or protein derived from a coronavirus, e.g., an RBD of an S protein, can be conjugated to the cell targeting molecule via a linker. In certain embodiments, the linker comprises Gly and Ser. In certain embodiments, the linker can, for example, and not by way of limitation, be between about 1 and about 25 or between about 5 and about 20 or between about 5 and about 15 amino acids in length. Non-limiting examples of linkers for use in the presently disclosure subject matter are disclosed in International Patent Publication WO 2017/165464 (e.g., SEQ ID NOs: 42, 44, 45, 75, 76, 77 and 78), the contents of which are incorporated by reference herein in its entirety.

In certain embodiments, the linker comprises a flexible linker. In certain embodiments, the linker comprises Gly (G) and Ser (S) amino acids. In certain embodiments, the flexible linker comprises a (G4S)3 linker, which corresponds to GGGGSGGGGSGGGGS (SEQ ID NO: 29). In certain embodiments, the linker comprises the amino acid sequence SEQ ID NO: 29 or that sequence bearing about 1 or about 2 amino acid substitutions, insertions or deletions, or about 1, about 2 or about 3 conservative amino acid substitutions. In certain embodiments, the antigen is a fusion protein comprising the amino acid sequence set forth in SEQ ID NO: 15, 16, 17 or 18 and the amino acid sequence set forth in SEQ ID NO: 26 linked by a linker comprising the amino acid sequence of SEQ ID NO: 29.

In certain embodiments, the linker can a rigid linker. In certain embodiments, the linker can be Pro-rich linker such as PAPAP (SEQ ID NO: 30). In certain embodiments, the linker comprises the amino acid sequence SEQ ID NO: 30 or that sequence bearing about 1 or about 2 amino acid substitutions, insertions or deletions, or about 1, about 2 or about 3 conservative amino acid substitutions. In certain embodiments, the rigid linker can be a helical linker such as AEAAAKEAAAKA (SEQ ID NO: 31) or A(EA₃K)₄AAA, which corresponds to AEAAAKEAAAKEAAAKEAAAKAAA (SEQ ID NO: 32). In certain embodiments, the linker comprises the amino acid sequence SEQ ID NO: 31 or 32 or that sequence bearing about 1 or about 2 amino acid substitutions, insertions or deletions, or about 1, about 2 or about 3 conservative amino acid substitutions.

In certain embodiments, the antigen can further comprise a His-tag. For example, but not by way of limitation, the antigen can comprise a His-tag at the C-terminus or N-terminus of the peptide or protein derived from a coronavirus. In certain embodiments, the His-tag can be a 6×His-tag (HHHHHH; SEQ ID NO: 33) or a 10×His-tag (HHHHHHHHHH; SEQ ID NO: 34). In certain embodiments, a 6×His-tag can be encoded by the nucleotide sequence CATCATCACCACCACCAT (SEQ ID NO: 35).

In certain embodiments, the antigen, expressed by a genetically-engineered cell of the present disclosure is secretable. For example, but not by way of limitation, the antigen can be expressed intracellularly in a cell and subsequently transported to the plasma membrane of the cell and secreted to the exterior of the cell, e.g., outside the plasma membrane of the cell. In certain embodiments, the antigen can be secreted using the mating secretory pathway. In certain embodiments, the antigen is displayed on the cell surface, e.g., plasma membrane, of the genetically-engineered cell. In certain embodiments, the antigen is not displayed on the surface, e.g., plasma membrane, of the genetically-engineered cell.

In certain embodiments, a genetically-engineered cell of the present disclosure can express and secrete an antigen disclosed herein and can further express an adjuvant. In certain embodiments, the adjuvant can be secreted from the genetically-engineered cell. In certain embodiments, a genetically-engineered cell disclosed herein can secrete an antigen and secrete an adjuvant. In certain embodiments, the adjuvant can be displayed on the cell surface of the genetically-engineered cell. In certain embodiments, a genetically-engineered cell disclosed herein can secrete an antigen and display an adjuvant on its surface.

In certain embodiments, a genetically-engineered cell of the present disclosure can express and secrete an antigen disclosed herein and can further express an adjuvant and a cell targeting molecule. In certain embodiments, the adjuvant and the cell targeting can both be secreted from the genetically-engineered cell. For example, but not by way of limitation, a genetically-engineered cell disclosed herein can secrete an antigen, secrete an adjuvant and secrete a cell targeting molecule. In certain embodiments, the adjuvant can be displayed on the cell surface of the genetically-engineered cell and/or the cell targeting molecule can be displayed on the cell surface of the genetically-engineered cell. For example, but not by way of limitation, a genetically-engineered cell disclosed herein can secrete an antigen, display an adjuvant on its surface and display a cell targeting molecule on its surface. Alternatively or additionally, a genetically-engineered cell disclosed herein can secrete an antigen, display an adjuvant on its surface and secrete a cell targeting molecule. In certain embodiments, a genetically-engineered cell disclosed herein can secrete an antigen, secrete an adjuvant and display a cell targeting molecule on its surface.

### III. Genetically-Engineered Cells

The present disclosure provides cells for expressing, e.g., secreting, an antigen derived from a coronavirus disclosed herein. For example, but not by way of limitation, cells of the present disclosure can include a nucleic acid that encodes one or more antigens. Non-limiting examples of antigens that can be produced by the cells of the present disclosure are disclosed in Section II. In certain embodiments, a genetically-engineered cell can express one or more antigens derived from a coronavirus, e.g., SARS-CoV-2.

The cells used for generating and/or secreting an antigen described herein can be, e.g., genetically-engineered cells. The genetically-engineered cell can be a mammalian cell, a plant cell, a bacterial cell or a fungal cell. For example, but not by way of limitation, the cell can be a mammalian cell, e.g., a genetically-engineered mammalian cell. In certain embodiments, the cell can be a plant cell, e.g., a genetically-engineered plant cell. In certain embodiments, the cell can be a bacterial cell, e.g., a genetically-engineered bacterial cell. In certain embodiments, the cell can be a fungal cell, e.g., a genetically-engineered fungal cell.

Any fungal strain can be used in the present disclosure. In certain embodiments, the fungal cell can be a species from a genus including, but not limited to, Cladosporium, Aureobasidium, Aspergillus, Saccharomyces, Malassezia, Epicoccum, Candida, Penicillium, Wallemia, Pichia, Phoma, Cryptococcus, Fusarium, Clavispora, Cyberlindnera, Kluyveromyces, Hansenula, Yarrowia, Neurospora, Schizosaccharomyces, Blastobotrys, Zygosaccharomyces, Debaryomyces, Torulaspora, Hanseniaspora, Rhodotorula, Wickerhamomyces and Williopsis.

In certain embodiments, a genetically-engineered cell of the present disclosure can be a cell of Alternaria brasicicola, Arthrobotrys oligospora, Ashbya aceri, Ashbya gossypii, Aspergillus clavatus, Aspergillus flavus, Aspergillus fumigate, Aspergillus kawachii, Aspergillus nidulans, Aspergillus niger, Aspergillus oryzae, Aspergillus ruber, Aspergillus terreus, Baudoinia compniacensis, Beauveria bassiana, Botryosphaeria parva, Botrytis cinereal, Candida albicans, Candida dubliniensis, Candida glabrata, Candida guilliermondii, Candida lusitaniae, Candida parapsilosis, Candida tenuis, Candida tropicalis, Capronia coronate, Capronia epimyces, Chaetomium globosum, Chaetomium thermophilum, Chryphonectria parasitica, Claviceps purpurea, Coccidioides immitis, Colletotrichum gloeosporioides, Coniosporium apollinis, Dactylellina haptotyla, Debaryomyces hansenii, Endocarpon pusillum, Eremothecium cymbalariae, Fusarium oxysporum, Fusarium pseudograminearum, Gaeumannomyces graminis, Geotrichum candidum, Gibberella fujikuroi, Gibberella moniliformis, Gibberella zeae, Glarea lozoyensis, Grosmannia clavigera, Kazachstania Africana, Kazachstania naganishii, Kluyveromyces lactis, Kluyveromyces marxianus, Kluyveromyces waltii, Komagataella pastoris, Kuraishia capsulate, Lachancea kluyveri, Lachancea thermotolerans, Lodderomyces elongisporus, Magnaporthe oryzae, Magnaporthe poae, Marssonina brunnea, Metarhizium acridum, Metarhizium anisopliae, Mycosphaerella graminicola, Mycosphaerella pini, Nectria haematococca, Neosartorya fischeri, Neurospora crassa, Neurospora tetrasperma, Ogataea parapolymorpha, Ophiostoma piceae, Paracoccidioides lutzii, Penicillium chrysogenum, Penicillium digitatum, Penicillium oxalicum, Penicillium roqueforti, Phaeosphaeria nodorum, Pichia sorbitophila, Podospora anserine, Pseudogymnoascus destructans, Pyrenophora teres teres, Pyrenophora tritici-repentis, Saccharomyces bayanus, Saccharomyces castellii, Saccharomyces cerevisiae, Saccharomyces dairenensis, Saccharomyces mikatae, Saccharomyces paradoxis, Scheffersomyces stipites, Schizosaccharomyces japonicus, Schizosaccharomyces octosporus, Schizosaccharomyces pombe, Sclerotinia borealis, Sclerotinia sclerotiorum, Sordaria macrospora, Sporothrix schenckii, Tetrapisispora blattae, Tetrapisispora phaffii, Thielavia heterothallica, Togninia minima, Torulaspora delbrueckii, Trichoderma atroviridis, Trichoderma jecorina, Trichoderma vixens, Tuber melanosporum, Vanderwaltozyma polyspora 1, Vanderwaltozyma polyspora 2, Verticillium alfalfae, Verticillium dahliae, Wickerhamomyces ciferrii, Yarrowia lipolytica, Zygosaccharomyces bailii, Zygosaccharomyces rouxii and combinations thereof.

In certain embodiments, the genetically engineered cell of the present disclosure is a species of phylum Ascomycota. In certain embodiments, the species of the phylum Ascomycota is selected from Saccharomyces cerevisiae, Saccharomyces castellii, Saccharomyces var boulardii, Vanderwaltozyma polyspora, Torulaspora delbrueckii, Saccharomyces kluyveri, Kluyveromyces lactis, Zygosaccharomyces rouxii, Zygosaccharomyces bailii, Candida glabrata, Ashbya gossypii, Scheffersomyces stipites, Komagataella (Pichia) pastoris, Candida (Pichia) guilliermondii, Candida parapsilosis, Candida auris, Yarrowia lipolytica, Candida (Clavispora) lusitaniae, Candida albicans, Candida tropicalis, Candida tenuis, Lodderomyces elongisporous, Geotrichum candidum, Baudoinia compniacensis, Schizosaccharomyces octosporus, Tuber melanosporum, Aspergillus oryzae, Schizosaccharomyces pombe, Aspergillus (Neosartorya) fischeri, Pseudogymnoascus destructans, Schizosaccharomyces japonicus, Paracoccidioides brasiliensis, Mycosphaerella graminicola, Penicillium chrysogenum, Aspergillus nidulans, Phaeosphaeria nodorum, Hypocrea jecorina, Botrytis cinereal, Beauvaria bassiana, Neurospora crassa, Sporothrix scheckii, Magnaporthe oryzea, Dactylellina haptotyla, Fusarium graminearum, Capronia coronate, Pichia methanolica, Pichia stipites and combinations thereof.

In certain embodiments, the genetically-engineered cell of the present disclosure is Saccharomyces cerevisiae, Saccharomyces boulardii, Saccharomyces mikatae, Saccharomyces kudriavzevii, Saccharomyces uvarum, Kluyveromyces lactis, Kluyveromyces marxianus, Kluyveromyces dobzhansky, Hansenula polymorpha, Yarrowia lipolytica, Neurospora crassa, Schizosaccharomyces pombe, Blastobotrys (Arxula) adeninivorans, Candida boldmu, Candida boidinii, Pichia pastoris, Pichia methanolica, Pichia stipites, Zygosaccharomyces rouxii, Zygosaccharomyces bailii and Schwanniomyces (Debaryomyces) occidentalis.

In certain embodiments, the genetically-engineered cell of the present disclosure is Saccharomyces cerevisiae, e.g., Saccharomyces cerevisiae S288C or Saccharomyces cerevisiae Fleischman's yeast. In certain embodiments, the genetically-engineered cell of the present disclosure is Saccharomyces cerevisiae S288C. In certain embodiments, the genetically-engineered cell of the present disclosure is Saccharomyces cerevisiae Fleischman's yeast.

In certain embodiments, the genetically-engineered cell of the present disclosure is Saccharomyces boulardii, e.g., Saccharomyces boulardii YM5016 or Saccharomyces boulardii CNCM I-745. In certain embodiments, the genetically-engineered cell is Saccharomyces boulardii YM5016. In certain embodiments, the genetically-engineered cell is Saccharomyces boulardii CNCM I-745.

In certain embodiments, the genetically-engineered cell of the present disclosure is Kluyveromyces lactis, e.g., Kluyveromyces lactis GG799. In certain embodiments, the genetically-engineered cell is Kluyveromyces lactis GG799.

In certain embodiments, the genetically-engineered cell of the present disclosure is a bacterial cell. Non-limiting examples of bacteria include Caulobacter crescentus, Rodhobacter sphaeroides, Pseudoalteromonas haloplanktis, Shewanella sp. strain Ac10, Pseudomonas fluorescens, Pseudomonas aeruginosa, Halomonas elongata, Chromohalobacter salexigens, Streptomyces lividans, Streptomyces griseus, Nocardia lactamdurans, Mycobacterium smegmatis, Corynebacterium glutamicum, Corynebacterium ammoniagenes, Brevibacterium lactofermentum, Bacillus subtilis, Bacillus brevis, Bacillus megaterium, Bacillus licheniformis, Bacillus amyloliquefaciens, Lactococcus lactis, Lactobacillus plantarum, Lactobacillus casei, Lactobacillus reuteri, Lactobacillus gasseri and Escherichia coli. In certain embodiments, the bacteria cell is Escherichia coli.

In certain embodiments, the genetically-engineered cell of the present disclosure is a mammalian cell. Non-limiting examples of mammalian cells include monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; FS4 cells; MCF-7 cells; 3T3 cells; U2SO cells; Chinese hamster ovary (CHO) cells' and myeloma cell lines such as Y0, NS0 and Sp2/0.

In certain embodiments, nucleic acids of the present disclosure encoding one or more of the antigens disclosed herein and/or one or more of the cell targeting molecules disclosed herein can be introduced into cells, e.g., yeast cells, using vectors, such as plasmid vectors and cell transformation techniques such as electroporation, heat shock, lithium acetate (Li-acetate) and others known to those skilled in the art and described herein. In certain embodiments, the genetic molecular components are introduced into the cell to persist as a plasmid or integrate into the genome. For example, but not by way of limitation, the nucleic acid can be incorporated into the genome of the genetically-engineered cell. In certain embodiments, the cells can be engineered to chromosomally integrate a polynucleotide of one or more genetic molecular components described herein, using methods identifiable to skilled persons upon reading the present disclosure. In certain embodiments, a nucleic acid encoding an antigen can be inserted into the genome of a genetically engineered cell using homologous recombination. In certain embodiments, a nucleic acid encoding an antigen of the present disclosure can be inserted into the genome of a genetically engineered cell using a Clustered regularly-interspaced short palindromic repeats (CRISPR)/Cas, e.g., CRISPR/Cas9 system.

In certain embodiments, a nucleic acid encoding one or more antigens disclosed herein and/or one or more of the cell targeting molecules disclosed herein can be introduced into cells is introduced into the yeast cell either as a construct or a plasmid. In certain embodiments, a nucleic acid can comprise one or more regulatory regions such as promoters, transcription factor binding sites, operators, activator binding sites, repressor binding sites, enhancers, protein-protein binding domains, RNA binding domains, DNA binding domains, and other control elements known to a person skilled in the art. For example, but not by way of limitation, a nucleic acid encoding an antigen is introduced into the yeast cell either as a construct or a plasmid in which it is operably linked to a promoter active in the yeast cell or such that it is inserted into the yeast cell genome at a location where it is operably linked to a suitable promoter. Non-limiting examples of suitable yeast promoters include, but are not limited to, constitutive promoters pTef1, pPgk1, pCyc1, pAdh1, pKex1, pTdh3, pTpi1, pPyk1 and pHxt7 and inducible promoters pGal1, pCup1, pMet15, pFig1, pFus1, GAP, P GCW14 and variants thereof. In certain embodiments, a variant of Tef1 is scTef1. In certain embodiments, a nucleic acid can include a constitutively active promoter, e.g., pTdh3. In certain embodiments, a nucleic acid can include an inducible promoter, e.g., pFus1 or pFig1. In certain embodiments, a nucleic acid can include a constitutively active promoter, e.g., pAdh1 or LAC4p. In certain embodiments, a nucleic acid can include a constitutively active promoter, e.g., pCyc1.

In certain embodiments, a nucleic acid encoding one or more of the antigens disclosed herein and/or one or more of the cell targeting molecules disclosed herein can further include a transcription factor for regulation expression of the molecule encoded by the nucleic acid. Alternatively and/or additionally, a second nucleic or an additional nucleic acid can be introduced into the cells to express a transcription factor for regulation expression of the antigen encoded by the nucleic acid. Non-limiting examples of such transcription factors include Abf1p, Aca1p, Ace2p, Adr1p, Aft1p, Aft2p, Arg80p, Arg81p, Arr1p, Ash1p, Azf1p, Bas1p, Cad1p, Cat8p, Cbf1p, Cha4p, Cha4p, Cin5p, Com2p, Crz1p, Cst6p, Cup2p, Dal80p, Dal81p, Dal82p, Ecm22p, Fkh1p, Fkh2p, Flo8p, Fzf1p, Gal4p, Gat1p, Gcn4p, Gcr1p, Gis1p, Gln3p, Gon3p, Gsm1p, Gzf3p, Haa1p, Haa1p, Hap1p, Hap2p, Hap3p, Hap4p, Hap5p, Hcm1p, Hot1p, Hsf1p, Ime1p, Ino2p, Ino4p, Ino4p, Ixr1p, Kar4p, Leu3p, Lys14p, Mac1p, Mal63p, Mbp1p, Mcm1p, Met31p, Met32p, Met4p, Mig1p, Mig2p, Mig3p, Mot2p, Mot3p, Msn2p, Msn4p, Mss11p, Ndt80p, Nrg1p, Nrg2p, Oaf1p, Pdr1p, Pdr3p, Pdr1p, Pho2p, Pho4p, Pip2p, Ppr1p, Put3p, Rap1p, Rcs1p, Rds1p, Reb1p, Rfx1p, Rgt1p, Rim101p, Rlm1p, Rme1p, Rof1p, Rox1p, Rph1p, Rpn4p, Rtg1p, Rtg3p, Sfl1p, Sip4p, Skn7p, Sko1p, Smp1p, Stb4p, Stb5p, Stb5p, Ste12p, Stp1p, Stp2p, Sum1p, Swi4p, Swi5p, Tda9p, Tea1p, Tec1p, Tye7p, Uga3p, Ume6p, Upc2p, Usv1p, War1p, Xbp1p, YER130c, YFL052w, YHR177w, YJL103C, YML081w, YPL230w, Yap1p, Yap3p, Yap5p, Yrr1p, Zap1p and Znf1p. In certain embodiments, a nucleic acid introduced into a genetically-engineered cell of the present disclosure includes one or more DNA binding domains for a transcription factor. In certain embodiments, the DNA binding domain is a zinc finger DNA binding domain. In certain embodiments, the zinc finger DNA binding domain is ZF43-8. In certain embodiments, the transcription factor comprises one or more domains from different proteins. For example, but not by way of limitation, a transcription factor for use in the present disclosure can include an inducer binding domain, e.g., a β-estradiol binding domain, e.g., derived from the human estrogen receptor, and/or a transcription activation domain, e.g., derived from VP64.

In certain embodiments, a nucleic acid encoding one or more antigens disclosed herein and/or one or more of the cell targeting molecules disclosed herein can be inserted into the genome of the cell, e.g., yeast cell. For example, but not by way of limitation, one or more nucleic acids encoding a molecule of the present disclosure, e.g., peptide and/or protein, can be inserted into the Ste2, Ste3 and/or HO locus of the cell. In certain embodiments, the one or more nucleic acids can be inserted into one or more loci that minimally affects the cell, e.g., in an intergenic locus or a gene that is not essential and/or does not affect growth, proliferation and cell signaling.

In certain embodiments, one or more endogenous genes of the genetically-engineered cells can be knocked out and/or mutated, e.g., knocked out by a genetic engineering system. Alternatively or additionally, one or more endogenous genes of the genetically-engineered cells can be replaced with a homolog from a different species. In certain embodiments, a genetically-engineered cell can be modified to include multiple copies of an endogenous gene to increase expression of the gene. Various genetic engineering systems known in the art can be used. Non-limiting examples of such systems include the CRISPR/Cas system, the zinc-finger nuclease (ZFN) system, the transcription activator-like effector nuclease (TALEN) system, use of yeast endogenous homologous recombination and the use of interfering RNAs.

In certain non-limiting embodiments, a CRISPR/Cas9 system is employed to knock out one or more endogenous genes in the genetically engineered cell. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9) and trans-activating crRNA (tracrRNA, binds to crRNA and forms an active complex with Cas9). The terms “guide RNA” and “gRNA” refer to any nucleic acid that promotes the specific association (or “targeting”) of an RNA-guided nuclease such as a Cas9 to a target sequence such as a genomic or episomal sequence in a cell. gRNAs can be unimolecular (comprising a single RNA molecule and referred to alternatively as chimeric) or modular (comprising more than one, and typically two, separate RNA molecules, such as a crRNA and a tracrRNA, which are usually associated with one another, for instance by duplexing).

In certain embodiments, a sequence homolog of a nucleotide sequence disclosed herein can be a polynucleotide having changes in one or more nucleotide bases that can result in substitution of one or more amino acids, but do not affect the functional properties of the polypeptide or protein encoded by the nucleotide sequence. Homologs can also include polynucleotides having modifications such as deletion, addition or insertion of nucleotides that do not substantially affect the functional properties of the resulting polynucleotide or transcript. Alterations in a polynucleotide that result in the production of a chemically equivalent amino acid at a given site, but do not affect the functional properties of the encoded polypeptide, are well known in the art.

In certain embodiments, a sequence homolog of an antigen disclosed herein can be a peptide, polypeptide or protein having changes in one or more amino acids but do not affect the functional properties of the peptide, polypeptide or protein. Alterations in a peptide, polypeptide or protein that do not affect the functional properties of the peptide, polypeptide or protein, are well known in the art, e.g., conservative substitutions. It is therefore understood that the disclosure encompasses more than the specific exemplary polynucleotide or amino acid sequences and includes functional equivalents thereof.

The cells to be used in the present disclosure can be genetically engineered using recombinant techniques known to those of ordinary skill in the art. Production and manipulation of the polynucleotides described herein are within the skill in the art and can be carried out according to recombinant techniques described, for example, in Sambrook et al. 1989. Molecular Cloning: A Laboratory Manual, 2d ed., Cold Spring Harbor Laboratory Press, Cold Spring Harbor, N.Y. and Innis et al. (eds). 1995. PCR Strategies, Academic Press, Inc., San Diego.

In certain embodiments, the genetically-engineered cells express and/or secrete an antigen disclosed herein at high levels as compared to previous known expression systems. For example, but not by way of limitation, the total titer of the antigen produced by a genetically-engineered cell, e.g., a population of genetically-engineered cells, is between about 1 pg and about 10 g, e.g., about 1 pg/L to about 10 g/L, about 1 pg/L to about 10 mg/L or 1 mg/L to about 100 mg/L.

In certain embodiments, a genetically-engineered cell of the present disclosure can comprise a nucleic acid that encodes an antigen comprising the amino acid sequence set forth in SEQ ID NO: 15, 16, 17 or 18 and the amino acid sequence set forth in SEQ ID NO: 26 linked by a linker comprising the amino acid sequence of SEQ ID NO: 29. In certain embodiments, a genetically-engineered cell of the present disclosure can comprise a nucleic acid that encodes an antigen comprising the amino acid sequence set forth in SEQ ID NO: 15, 16, 17 or 18 and the amino acid sequence set forth in SEQ ID NO: 26 linked by a linker comprising the amino acid sequence of SEQ ID NO: 29.

In certain embodiments, a genetically-engineered cell of the present disclosure can comprise a nucleic acid, e.g. a first nucleic acid, that encodes an antigen comprising the amino acid sequence set forth in SEQ ID NO: 15, 16, 17 or 18. In certain embodiments, the nucleic acid, e.g. a first nucleic acid, can further encode a cell targeting molecule. Alternatively or additionally, the genetically-engineered cell of the present disclosure can comprise a second nucleic acid that encodes a cell targeting molecule. In certain embodiments, the cell targeting molecule can comprise an amino acid sequence set forth in SEQ ID NO: 24, 26, 28, 37, 39, 41 or 43. In certain embodiments, the first and/or second or a third nucleic acid can be introduced in the genetically-engineered cell to express an adjuvant.

### IV. Methods of Use

The present disclosure further provides methods for using the genetically-engineered cells of the present disclosure. The presently disclosed matter provides methods for protecting a subject from coronavirus. For example, but not by way of limitation, the presently disclosed subject matter provides methods for protecting a subject from SARS-CoV-2. In certain embodiments, the present disclosure provides methods for preventing, minimizing, reducing and/or alleviating an infection, e.g., preventing and/or alleviating a coronavirus infection. In certain embodiments, the present disclosure provides methods for preventing and/or reducing the severity of a coronavirus infection in a subject.

In certain embodiments, the coronavirus is an alpha-coronavirus, a beta-coronavirus, a gamma-coronavirus or a delta-coronavirus. In certain embodiments, the coronavirus is an alpha-coronavirus. In certain embodiments, the coronavirus is a beta-coronavirus. In certain embodiments, the coronavirus is a gamma-coronavirus. In certain embodiments, the coronavirus is a delta-coronavirus. In certain embodiments, the coronavirus is an alpha-coronavirus such as, but not limited to, the coronavirus strains HCoV-229E and HCoV-NL63. In certain embodiments, the coronavirus is a beta-coronavirus such as, but not limited to, the coronavirus strains HCoV-0C43, HCoV-HKU1, MERS-CoV and SARS-CoV. In certain embodiments, the coronavirus is MERS-CoV. In certain embodiments, the coronavirus is SARS-CoV.

In certain embodiments, the coronavirus is SARS-CoV-2. In certain embodiments, the coronavirus can be a variant of SARS-CoV-2. In certain embodiments, the SARS-CoV-2 variant can be an alpha variant, a beta variant, a gamma variant, an epsilon variant, a kappa variant, an iota variant, an eta variant, a lambda variant or a zeta variant. Non-limiting examples of SARS-CoV-2 variants include the B.1.1.7 (alpha), B.1.351 (beta), P.1 (gamma), B.1.427 (epsilon), B.1.429 (epsilon), B.1.617.2 (delta), B.1.526.1 (iota), B.1.526.2 (iota), B.1.525 (eta), P.2 (zeta) and B.1.526 (iota) variants. In certain embodiments, the coronavirus is the SARS-CoV-2 B.1.1.7 variant. In certain embodiments, the coronavirus is the SARS-CoV-2 B.1.351 variant. In certain embodiments, the coronavirus is the SARS-CoV-2 P.1 variant. In certain embodiments, the coronavirus is the SARS-CoV-2 B.1.427 variant. In certain embodiments, the coronavirus is the SARS-CoV-2 B.1.429 variant. In certain embodiments, the coronavirus is the SARS-CoV-2 B.1.617.2 variant.

In certain embodiments, the methods of the present disclosure include administering one or more genetically-engineered cells of the present disclosure, e.g., a population of genetically-engineered cells of the present disclosure. In certain embodiments, the genetically-engineered cell administered to a subject generates and secretes an antigen for inducing an immune response in the subject. Non-limiting examples of antigens that can be generated and secreted are disclosed herein in Section II. For example, but not by way of limitation, the antigen comprises a peptide or protein or fragment thereof derived from a coronavirus, e.g., SARS-CoV-2. In certain embodiments, the antigen comprises an S protein or fragment thereof from SARS-CoV-2. In certain embodiments, the antigen comprises an RBD of an S protein, e.g., from SARS-CoV-2. In certain embodiments, the antigen is a fusion protein that comprises an S protein or fragment thereof, e.g., an RBD of a S protein from SARS-CoV-2, and a cell targeting molecule, e.g., CTB.

In certain embodiments, a method of the present disclosure includes administering one or more live and/or intact genetically-engineered cells, e.g., fungal cells, expressing one or more antigens described herein. In certain embodiments, a live genetically-engineered cell refers to a cell that has an intact cell membrane and has one or more of the following properties: (1) is actively dividing, (2) is metabolically active and/or (3) actively expresses a therapeutic molecule. In certain embodiments, the genetically-engineered cells are not killed, e.g., heat-killed, prior to administration.

The methods described herein provides a more cost-effective method for administering an antigen to a subject in need thereof without requiring the purification of the antigen from the genetically-engineered cell prior to administration to the subject. For example, but not by way of limitation, the generation of an antigen by a genetically-engineered cell in situ and administration of such a cell can avoid, prevent and/or reduce the degradation of the antigen that can occur during the manufacturing, purification and/or storing processes, and can avoid, prevent and/or reduce the degradation of the antigen after administration to a patient. The compositions of the genetically-engineered cells disclosed herein will be a vaccine that is extra low cost, highly scalable, easy to produce and formulate, does not require cold-chain transport and storage, does not require needles or trained personnel to administer and will be available globally including resource-poor countries. Additional benefits of the presently disclosed genetically-engineered cells for use as a vaccine against a coronavirus include the ability of the yeast to stimulate an immune response separately from the secreted antigen, which can potentiate the immune response against the antigen. Also the ability to administer the cells by intranasal or gastrointestinal administration will elicit a mucosal immunity, which is important as mucosae are the main entry site of coronaviruses.

In certain embodiments, a method of the present disclosure includes administering to the subject in need thereof a cell genetically engineered to generate and secrete an antigen for inducing an immune response in the subject or a pharmaceutical composition thereof. In certain embodiments, the immune response can be a mucosal, humoral and/or cellular immune response.

In certain embodiments, the genetically-engineered cell is a fungal cell that produces an antigen in situ and secretes the antigen. The genetically-administered cell or a pharmaceutical composition thereof can be administered to the subject by any method. For example, but not by way of limitation, the genetically-engineered cell can be administered by parenteral administration, intraocular administration, intraaural administration, enteral administration, intranasal administration, oral administration, rectal administration, vaginal administration or topical administration. In certain embodiments, the genetically-engineered cell is administered by enteral administration. In certain embodiments, the genetically-engineered cell is administered by intranasal administration. In certain embodiments, the genetically-engineered cell can be administered through a mucosal membrane, e.g., administered intranasally or orally.

In certain embodiments, a method of the present disclosure can further include administering the one or more genetically-engineered cells with a cell targeting molecule. Non-limiting examples of such cell targeting molecules are disclosed in Section II. For example, and not by way of limitation, the cell targeting molecule can be a CTB.

In certain embodiments, a genetically-engineered cell disclosed herein can be administered once a day, twice a day, once a week, twice a week, three times a week, four times a week, five times a week, six times a week, once every two weeks, once a month, twice a month, once every other month, once every third month, once every 6 months or once a year. In certain embodiments, the genetically-engineered cell can be administered twice a week. In certain embodiments, a genetically-engineered cell disclosed herein be administered once a week. In certain embodiments, a genetically-engineered cell disclosed herein can be administered every 10-14 days. In certain embodiments, a genetically-engineered cell disclosed herein can be administered two times a week for about four weeks and then administered once a week for the remaining duration of the treatment.

In certain embodiments, a method of the present disclosure can further include administering an adjuvant. Non-limiting examples of adjuvants include lipopolysaccharide (LPS), a Lipid A portion thereof, Bordetella pertussis, pertussis toxin, Mycobacterium tuberculosis, muramyl dipeptide (MDP), Freund's adjuvant, Freund's complete adjuvant (FCA), aluminum hydroxide adjuvant, Ribi adjuvants, Titermax adjuvants, specol adjuvants, aluminum salt adjuvants and monophosphoryl Lipid A-synthetic trehalose dicorynomylcolate (MPL-TDM).

### V. Pharmaceutical Compositions

The present disclosure further provides pharmaceutical compositions comprising a genetically-engineered cell for use according to the disclosed methods. In certain embodiments, the pharmaceutical compositions include one or more live and/or intact genetically-engineered cells, e.g., fungal cells, expressing one or more antigens and/or cell targeting molecules.

In certain embodiments, a pharmaceutical composition of the present disclosure comprises one or more live and/or intact genetically-engineered fungal cells expressing and secreting an antigen comprising a peptide or protein or fragment thereof derived from a coronavirus, e.g., SARS-CoV-2. For example, but not by way of limitation, the antigen comprises an S protein or fragment thereof, e.g., an RBD of an S protein, e.g., from SARS-CoV-2. In certain embodiments, the antigen is a fusion protein that comprises an S protein or fragment thereof, e.g., an RBD of a S protein from SARS-CoV-2, and a cell targeting molecule, e.g., CTB. Alternatively or additionally, the genetically-engineered fungal cell further expresses a cell targeting molecule, which can be secreted from the genetically-engineered fungal cell or displayed on the surface of the genetically-engineered fungal cell.

In certain embodiments, a pharmaceutical composition for use accordingly to the present disclosure can be formulated for parenteral administration, intraocular administration, intraaural administration, intranasal administration, oral administration, rectal administration, vaginal administration, enteral administration or topical administration. In certain embodiments, the pharmaceutical composition is formulated for enteral administration. In certain embodiments, the pharmaceutical composition is formulated for intranasal administration.

In certain embodiments, the pharmaceutical composition includes a genetically-engineered cell, disclosed herein, and a pharmaceutically acceptable carrier. “Pharmaceutically acceptable,” as used herein, includes any carrier which does not interfere with the effectiveness of the biological activity of the active ingredients, e.g., the genetically-engineered cell and/or the therapeutic molecule, and that is not toxic to the patient to whom it is administered. Non-limiting examples of suitable pharmaceutical carriers include phosphate-buffered saline solutions, water, emulsions, such as oil/water emulsions, various types of wetting agents and sterile solutions. Additional non-limiting examples of pharmaceutically acceptable carriers can include gels, bioabsorbable matrix materials, implantation elements containing the inhibitor and/or any other suitable vehicle, delivery or dispensing means or material. Such carriers can be formulated by conventional methods and can be administered to the subject.

In certain embodiments, a pharmaceutical composition of the present disclosure can include nutrients for promoting the growth of the one or more genetically-engineered cells present in the composition. For example, but not by way of limitation, a pharmaceutical composition can include vitamins, e.g., peptone, yeast extract, water-soluble vitamins, carbohydrates, e.g., glucose, peptides, amino acids and/or salts. In certain embodiments, the pharmaceutical composition can include growth media for the genetically-engineered cells. In certain embodiments, the growth media is a dry growth media. In certain embodiments, the growth media is a solid form of growth media, e.g., agar-based growth media. Additional non-limiting examples of media and components that can be present in the media to support growth of the genetically-engineered cells are disclosed in Hagerdal et al., Microbial Cell Factories 4:31 (2005), the contents of which are disclosed herein by reference in their entirety.

In certain embodiments, the pharmaceutical compositions suitable for use in the present disclosure can include compositions where the genetically-engineered cells are contained in a therapeutically effective amount. A “therapeutically effective amount” refers to an amount of genetically-engineered cells and/or antigen produced by the genetically-engineered cells that is able to induce an immune reaction in the subject to the antigen. The therapeutically effective amount of an antigen can vary depending on the formulation used, the condition and its severity, and the age, weight, etc., of the subject to be treated. In certain embodiments, a pharmaceutical composition can include one or more lyophilized genetically-engineered cells of the present disclosure.

In certain embodiments, the pharmaceutical compositions of the present disclosure can be formulated using pharmaceutically acceptable carriers well known in the art that are suitable for parenteral administration, e.g., intravenous administration, intraarterial administration, intrathecal administration, intranasal administration, intramuscular administration, subcutaneous administration and intracisternal administration. For example, but not by way of limitation, the pharmaceutical composition can be formulated as solutions, suspensions or emulsions.

In certain non-limiting embodiments, the pharmaceutical compositions of the present disclosure can be formulated using pharmaceutically acceptable carriers well known in the art that are suitable for intraocular, oral, intranasal, enteral or rectal administration. Such carriers enable the pharmaceutical compositions to be formulated as tablets, pills, capsules, liquids, gels, syrups, slurries, suspensions, suppositories and the like, for intraocular, oral, intranasal, enteral or rectal administration to the patient to be treated. In certain embodiments, the pharmaceutical composition for oral or enteral administration can be in the form of a pill or tablet. In certain embodiments, the pharmaceutical composition for intranasal administration can be formulated to be administered as a nasal spray.

In certain embodiments, the tablets, pills, capsules and the like can contain any of the following ingredients, or compounds of a similar nature: a binder such as microcrystalline cellulose, gum tragacanth or gelatin, an excipient such as starch or lactose, a disintegrating agent such as alginic acid, primogel, or corn starch, a lubricant such as magnesium stearate or sterotes, a glidant such as colloidal silicon dioxide, a sweetening agent such as sucrose or saccharin or a flavoring agent.

In certain embodiments, the pharmaceutical compositions can be prepared in the form of suppositories or retention enemas for rectal administration. In certain embodiments, the pharmaceutical compositions can be prepared with carriers that will protect the genetically-engineered cells against rapid elimination from the body, such as a controlled release formulation, including implants. In certain embodiments, biodegradable, biocompatible polymers can be used, such as ethylene vinyl acetate, polyanhydrides, polyglycolic acid, collagen, polyorthoesters and polylactic acid.

In certain embodiments, the pharmaceutical compositions of the present disclosure can be formulated using pharmaceutically acceptable carriers well known in the art that are suitable for topical administration. such carriers enable the pharmaceutical compositions to be formulated as liquids, gels, creams, syrups, slurries, dispersible powders, suspensions, lotions and the like, for topical administration to the patient to be treated.

In certain embodiments, the pharmaceutical composition can further include an adjuvant. Non-limiting examples of adjuvants include lipopolysaccharide (LPS), a Lipid A portion thereof, Bordetella pertussis, pertussis toxin, Mycobacterium tuberculosis, muramyl dipeptide (MDP), Freund's adjuvant, Freund's complete adjuvant (FCA), aluminum hydroxide adjuvant, Ribi adjuvants, Titermax adjuvants, specol adjuvants, aluminum salt adjuvants and monophosphoryl Lipid A-synthetic trehalose dicorynomylcolate (MPL-TDM).

### VI. Kits

The presently disclosed subject matter further provides kits comprising one of more genetically-engineered cells disclosed herein. For example, but not by way of limitation, a kit can include one or more live and/or intact genetically-engineered fungal cells expressing and secreting an antigen comprising a peptide or protein or fragment thereof derived from a coronavirus, e.g., SARS-CoV-2. In certain embodiments, the antigen comprises an S protein or fragment thereof, e.g., an RBD of an S protein, e.g., from SARS-CoV-2. In certain embodiments, the antigen is a fusion protein that comprises an S protein or fragment thereof, e.g., an RBD of a S protein from SARS-CoV-2, and a cell targeting molecule, e.g., CTB. Alternatively or additionally, the genetically-engineered fungal cell further expresses a cell targeting molecule, which can be secreted from the genetically-engineered fungal cell or displayed on the surface of the genetically-engineered fungal cell.

In certain embodiments, the genetically-engineered cells are formulated in a dried form. In certain embodiments, the genetically-engineered cells are lyophilized. In certain embodiments, the kit further includes an activator, e.g., water, to activate the genetically-engineered yeast prior to administration.

In certain embodiments, the genetically-engineered yeast can be formulated for intranasal administration, e.g., as a nasal spray. In certain embodiments, the genetically-engineered yeast can be formulated for oral administration, e.g., in a pill, e.g., in a freeze-dried form.

### VII. Exemplary Embodiments

A. In certain non-limiting embodiments, the presently disclosed subject matter provides for a fungal cell genetically engineered to produce an antigen comprising a protein or fragment thereof derived from a coronavirus in situ, wherein the antigen is secreted from the fungal cell.

A1. The genetically-engineered fungal cell of A, wherein the coronavirus is SARS-CoV-2, MERS-CoV or SARS-CoV.

A2. The genetically-engineered fungal cell of A-A1, wherein the coronavirus is SARS-CoV-2.

A3. The genetically-engineered fungal cell of any one of A-A2, wherein the coronavirus is a variant of SARS-CoV-2.

A4. The genetically-engineered fungal cell of A3, wherein the variant of SARS-CoV-2 is selected from the group consisting of an alpha variant, a beta variant, a gamma variant, a delta variant, an epsilon variant and a combination thereof.

A5. The genetically-engineered fungal cell of A3 or A4, the variant of SARS-CoV-2 is selected from the group consisting of B.1.1.7, B.1.351, P.1, B.1.427, B.1.429, B.1.617.2 and a combination thereof.

A6. The genetically-engineered fungal cell of any one of A-A5, wherein the antigen is secreted from the fungal cell by a secretory pathway of the fungal cell.

A7. The genetically-engineered fungal cell of any one of A-A6, wherein the antigen is secreted from the fungal cell by the alpha-mating factor secretion pathway of the fungal cell.

A8. The genetically-engineered fungal cell of any one of A-A7 wherein the antigen comprises an S protein or fragment thereof derived from the coronavirus.

A9. The genetically-engineered fungal cell of A8, wherein the S protein or fragment thereof is the receptor-binding domain (RBD) of the S protein.

A10. The genetically-engineered fungal cell of A9, wherein the RBD comprises one or more amino acid substitutions as shown in Table 3.

A11. The genetically-engineered fungal cell of A9 or A10, wherein the RBD comprises one or more amino acid substitutions to modify the glycosylation and disulfide bonds of the RBD.

A12. The genetically-engineered fungal cell of any one of A-A11, wherein the antigen comprises an amino acid sequence that is about 80%, about 85%, about 90% or about 95% homologous to an amino acid sequence set forth in SEQ ID NOs: 12-22.

A13. The genetically-engineered fungal cell of any one of A-A12, wherein the antigen comprises an amino acid sequence set forth in SEQ ID NOs: 12-22 or conservative substitutions thereof.

A14. The genetically-engineered fungal cell of any one of A-A13, wherein the antigen is encoded by a nucleotide sequence that is about 70%, about 75%, about 80%, about 85%, about 90% or about 95% homologous to a nucleotide sequence set forth in SEQ ID NOs: 1-11.

A15. The genetically-engineered fungal cell of any one of A-A14, wherein the antigen is encoded by a nucleotide sequence set forth in SEQ ID NOs: 1-11.

A16. The genetically-engineered fungal cell of any one of A-A15, wherein the genetically-engineered fungal cell further expresses and/or secretes a cell targeting molecule.

A17. The genetically-engineered fungal cell of any one of A-A16, wherein the antigen further comprises a cell targeting molecule.

A18. The genetically-engineered fungal cell of A16 or A17, wherein the cell targeting molecule is a molecule for targeting the genetically-engineered fungal cell and/or antigen to an immune cell.

A19. The genetically-engineered fungal cell of A18, wherein the immune cell is located in a mucosal membrane and/or located in the Peyer's patches in the small intestine.

A20. The genetically-engineered fungal cell of any one of A16-A19, wherein the cell targeting molecule is selected from the group consisting of a C-terminal Clostridium perfringens Enterotoxin (C-CPE), a Cholera Toxin Subunit B (CTB), a heat-labile enterotoxin B subunit (LTB), a C-terminal Clostridium difficile toxin A (TxA(C314)), CRM197 (a diphtheria toxin mutant), Fragment C of tetanus toxoid, a cholesteryl group-bearing pullulan (CHP), PT-9K/129G (a detoxified derivative of pertussis toxin) or mutants thereof and a combination thereof.

A21. The genetically-engineered fungal cell of any one of A16-A20, wherein the cell targeting molecule comprises a CTB.

A22. The genetically-engineered fungal cell of any one of A16-A21, wherein the cell targeting molecule is coupled to the protein or fragment thereof derived from a coronavirus by a linker, e.g., an N-terminal linker, a C-terminal linker or a combination thereof.

A23. The genetically-engineered fungal cell of A22, wherein the linker comprises Gly and Ser.

A24. The genetically-engineered fungal cell of any one of A-A23, wherein the genetically-engineered fungal cell further expresses and/or secretes an adjuvant.

A25. The genetically-engineered fungal cell of any one of A-A24, wherein the fungal cell is one or more species from a genus selected from the group consisting of Cladosporium, Aureobasidium, Aspergillus, Saccharomyces, Malassezia, Epicoccum, Candida, Penicillium, Wallemia, Pichia, Phoma, Cryptococcus, Fusarium, Clavispora, Cyberlindnera, Kluyveromyces, Hansenula, Yarrowia, Neurospora, Schizosaccharomyces, Blastobotrys, Zygosaccharomyces, Debaryomyces, Torulaspora, Hanseniaspora, Rhodotorula, Wickerhamomyces and Williopsis and a combination thereof.

A26. The genetically-engineered fungal cell of A25, wherein the fungal cell is selected from the group consisting of Saccharomyces cerevisiae, Saccharomyces boulardii, Saccharomyces mikatae, Saccharomyces kudriavzevii, Saccharomyces uvarum, Kluyveromyces lactis, Kluyveromyces marxianus, Kluyveromyces dobzhansky, Hansenula polymorpha, Yarrowia lipolytica, Neurospora crassa, Schizosaccharomyces pombe, Blastobotrys (Arxula) adeninivorans, Candida boldmu, Candida boidinii, Pichia pastoris, Pichia methanolica, Pichia stipites, Zygosaccharomyces rouxii, Zygosaccharomyces bailii and Schwanniomyces (Debaryomyces) occidentalis.

A27. The genetically-engineered fungal cell of A26, wherein the fungal cell is Saccharomyces cerevisiae, Saccharomyces boulardii and/or Kluyveromyces lactis.

B. A method for preventing and/or reducing the severity of a coronavirus infection in a subject comprising administering to the subject the genetically-engineered cell of any one of A-A27.

B1. The method of B, wherein the coronavirus is SARS-CoV-2, MERS-CoV or SARS-CoV.

B2. The method of B or B1, wherein the coronavirus is SARS-CoV-2.

B3. The method of any one of B-B2, wherein the coronavirus is a variant of SARS-CoV-2.

B4. The method of B3, wherein the variant of SARS-CoV-2 is selected from the group consisting of an alpha variant, a beta variant, a gamma variant, a delta variant, an epsilon variant and a combination thereof.

B5. The method of B3 or B4, the variant of SARS-CoV-2 is selected from the group consisting of B.1.1.7, B.1.351, P.1, B.1.427, B.1.429, B.1.617.2 and a combination thereof.

B6. The method of any one of B-B5, wherein the genetically-engineered fungal cell is formulated for parenteral administration, intraocular administration, intraaural administration, intranasal administration, oral administration, enteral administration, rectal administration, vaginal administration or topical administration.

B7. The method of B6, wherein the genetically-engineered fungal cell is formulated for enteral administration.

B8. The method of B6, wherein the genetically-engineered fungal cell is formulated for intranasal administration.

B9. The method of any one of B-B8, wherein the genetically-engineered fungal cell is administered once every two weeks.

B10. The method of any one of B-B9, further comprising the administration of an adjuvant.

C. A pharmaceutical composition comprising one or more genetically-engineered fungal cells of any one of A-A27 and a pharmaceutically acceptable carrier.

C1. The pharmaceutical composition of C, wherein the pharmaceutical composition is formulated for parenteral administration, intraocular administration, intraaural administration, intranasal administration, oral administration, enteral administration, rectal administration, vaginal administration or topical administration.

C2. The pharmaceutical composition of C1, wherein the pharmaceutical composition is formulated for enteral administration.

C3. The pharmaceutical composition of C1, wherein the pharmaceutical composition is formulated for intranasal administration.

C4. The pharmaceutical composition of any one of C-C3, wherein the pharmaceutical composition further comprises a cell targeting molecule.

C5. The pharmaceutical composition of any one of C-C4, wherein the pharmaceutical composition further comprises an adjuvant.

D. A kit comprising the genetically-engineered cell of any one of A-A27.

D1. A kit comprising the pharmaceutical composition of any one of C-C5.

E. The genetically-engineered cell of any one of A-A27 or the pharmaceutical compositions of any one of C-C5 for use in preventing and/or reducing the severity of a coronavirus infection.

E1. The genetically-engineered cell for use of E, wherein the coronavirus is SARS-CoV-2, MERS-CoV or SARS-CoV.

E2. The genetically-engineered cell for use of E1, wherein the coronavirus is SARS-CoV-2.

E3. The genetically-engineered cell for use of E2, wherein the coronavirus is a variant of SARS-CoV-2.

E4. The genetically-engineered cell for use of E3, wherein the variant of SARS-CoV-2 is selected from the group consisting of an alpha variant, a beta variant, a gamma variant, a delta variant, an epsilon variant and a combination thereof.

E5. The genetically-engineered cell for use of E3 or E4, the variant of SARS-CoV-2 is selected from the group consisting of B.1.1.7, B.1.351, P.1, B.1.427, B.1.429, B.1.617.2 and a combination thereof.

### EXAMPLES

The following examples are put forth so as to provide those of ordinary skill in the art with a complete disclosure and description of how to make and use the presently disclosed subject matter and are not intended to limit the scope of what the inventors regard as their presently disclosed subject matter. It is understood that various other implementations and embodiments can be practiced, given the general description provided herein.

### Example 1. Yeast Secreting RBD of SARS-CoV-2

The present Example discloses the generation and secretion of the RBD of the spike (S) protein of SAR-CoV-2 in yeast.

The RBD from the S protein of SARS-CoV-2 was cloned in a vector under the control of the constitutively active promoter pAdh1 as shown in FIG. 1 for transformation in S. cerevisiae and S. boulardii. For expression and secretion in K. lactis, the RBD from the S protein of SARS-CoV-2 was cloned in a vector under the control of the constitutively active LAC4p promoter in the presence of galactose as shown in FIG. 2. Mutants of the RBD of the S protein of SARS-CoV-2 to eliminate glycosylation sites and/or disulfide bonds were also cloned in a vector under the control of the constitutively active promoter pAdh1. The mutations that were generated in the RBD are shown in Table 4. The RBD-N1C1 (N331A, C538A) mutation was used to remove glycosylation sites.

S. cerevisiae, S. boulardii and K. lactis were transformed with one of the vectors using Li-acetate transformation. Transformed S. cerevisiae and S. boulardii were cultured in rich media YEPD for 2-3 days to secrete the SARS-CoV-2 antigen, and transformed K. lactis was cultured in rich media YPGal (YEP+40% Galactose) for 2-4 days to secrete the SARS-CoV-2 antigen.

TABLE 4

| No. | Site | N1 | N2 | C1 |
|---|---|---|---|---|
| 1 | N1 | N331A | | |
| 2 | N2 | | N343A | |
| 3 | N1N2 | N331A | N343A | |
| 4 | C1 | | | C538A |
| 5 | C1N1 | N331A | | C538A |
| 6 | C1N2 | | N343A | C538A |
| 7 | C1N1N2 | N331A | N343A | C538A |

The media was collected on the last day and spun down to collect the supernatant. The supernatant was concentrated using a 10 kDa cellulose filter (Millipore Sigma, cat. UFC901024) to 50× concentration, and reconstituted in Ni-NTA binding buffer. The reconstituted supernatant was then purified by a Ni-NTA column (Thermo Scientific, cat. 25215). The expressed antigen proteins from the supernatant and the Ni-NTA elution were detected by western-blot using a His-tag antibody (GenScript, cat. A00174-40) as shown in FIGS. 1 and 2.

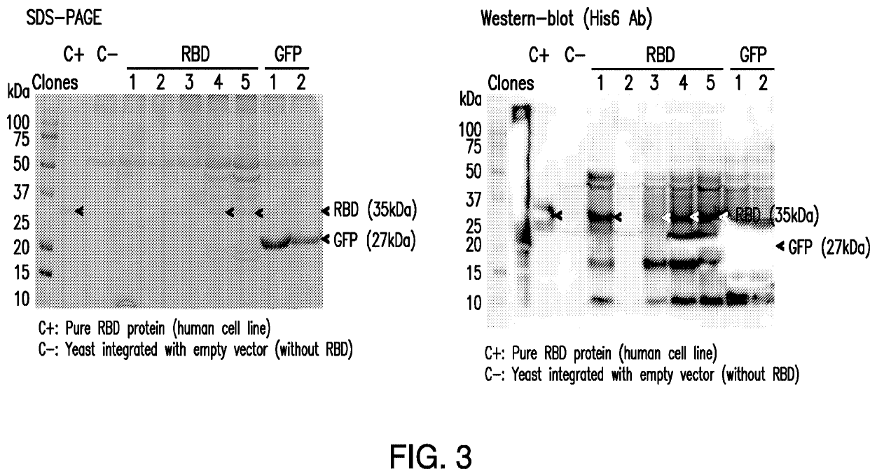

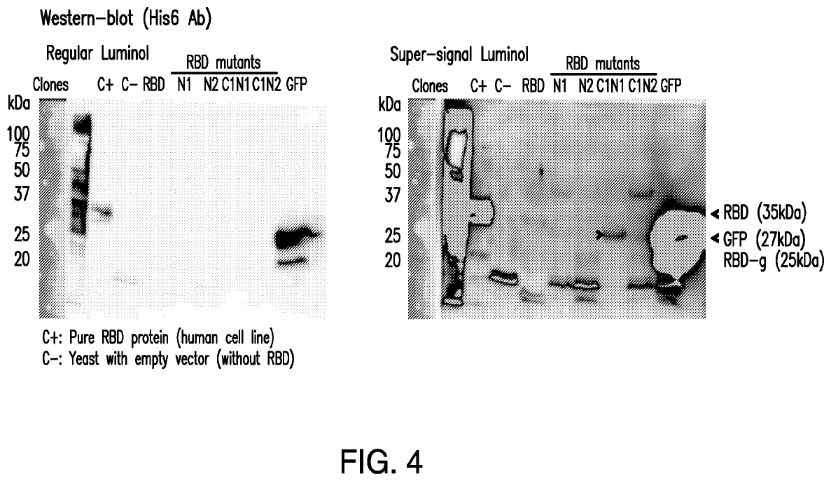

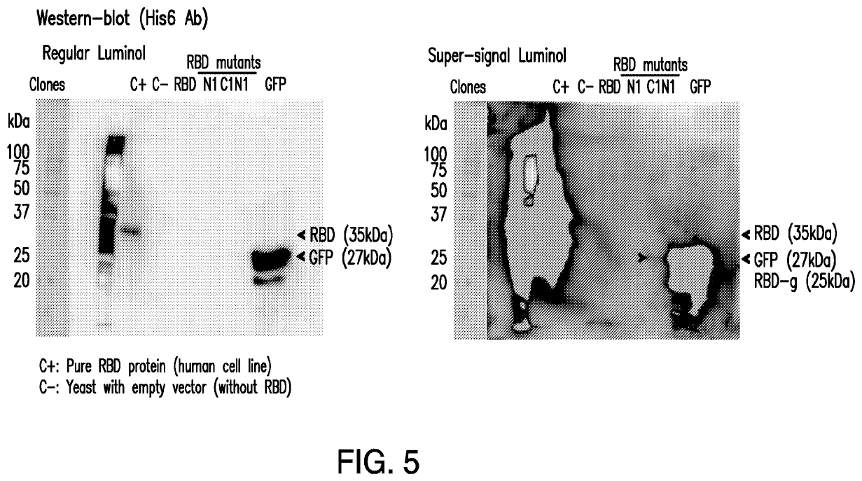

As shown in FIG. 3, K. lactis expressed and secreted the RBD, which included amino acids 319-541 of the S protein. A band around 35 kDa, which corresponds to the RBD secreted by the yeast, was observed by SDS-PAGE and Western Blot (FIG. 3). The C1N1 mutant of the RBD (amino acids 319-541 of the S protein, which includes mutations N331A and C538A) was expressed and secreted by S. cerevisiae and detected by Western Blot as shown in FIG. 4. Similarly, the C1N1 mutant of the RBD (amino acids 319-541 of the S protein, which includes mutations N331A and C538A) was expressed and secreted by S. boulardii and detected by Western Blot as shown in FIG. 5.

As shown this Example and FIGS. 3-5, the RBD and mutants of the RBD were expressed and secreted by various yeast species.

### Example 2. Immunogenicity of the RBD of SARS-CoV-2 Secreted from Yeast

The Example describes the immunogenicity of the RBD proteins secreted from the transformed yeast.

Secreted RBD proteins eluted from Ni-NTA columns as described in Example 1 will then be purified by size-exclusion chromatography. The purified RBD protein is then digested with trypsin on the Investigator ProGest (Genomic Solutions) automated digester, and analyzed in a LC-MS/MS spectrometer (Q Exactive™ Tandem Mass Spectrometry) and searched against databases (Mascot, Sequest HT) and specific antigen sequences (NCBI ID: 43740568) to confirm the identity of the protein. The exact identity of the protein is further confirmed by an N-terminal sequencing experiment (Edman degradation).

The purified antigen protein will be analyzed for immunogenicity by surface plasmon resonance (SPR). Briefly, the purified antigen is loaded on to a sensor chip and the binding affinity (k_{D}) to various SARS-CoV-2 neutralizing antibodies (3-10 antibodies whose epitopes cover different region of the antigen) are measured by the Biacore™ SPR machine.

The binding constant of the yeast recombinant antigen is compared to a human cell line-expressed antigen, and the antigenicity of the yeast-secreted antigen is promising if the binding constant is similar (within 1 magnitude) to the human antigen and will show that the derived protein is determined to be antigenic.

### Example 3: Induction of an Immune Response by Administration of Genetically-Engineered Yeast

The Example describes the measurement of the increase in humoral and cellular response to RBD in yeast-treated mice compared to negative control mice. Yeast expressing different RBDs will be tested including those discussed in Example 1.

The mice to be used in the experiments described in this Example are adult BALB/c mice, aged 2-4 months. The mice will be treated with antibiotics (penicillin and streptomycin) delivered in the drinking water starting from 3-4 days prior to the yeast administration. A group of mice will not receive any antibiotics, to allow the measurement of the yeast's viability in mice with an unaltered gastrointestinal microbiome.

Different experimental groups will be used including (1) mice administered yeast that secrete RBD, (2) mice administered yeast displaying RBD on their surface, (3) mice administered yeasts that were not genetically engineered as a negative control and (4) mice administered yeast expressing ovalbumin-CPE as a positive control.

To test intranasal and oral administration, live yeast is delivered directly into the stomach of the mice through oral gavage on day 0 and then again by oral gavage at day 14 for a booster dose or administrated intranasally on day 0 and then again by intranasal administration at day 14 for a booster dose. Stool and blood are then collected periodically from the mice starting from day 0 for the duration of the experiment. Stool is resuspended and homogenized in water, and then cultured on selective plates to detect the presence of live yeasts in the gastrointestinal tract and also analyzed with ELISA to measure the production of antibodies against RBD (humoral response).

At day 28, mice are sacrificed, and their blood and spleen are extracted to be analyzed for immune responses against RBD. Blood is analyzed with ELISA as previously stated. T cells are isolated from the spleen and the number of T cells reactive to RBD is then quantified by ELISpot (cellular response).

### REFERENCES

1. Baeshen N A, Baeshen M N, Sheikh A, Bora R S, Ahmed M M, Ramadan H A, Saini K S, Redwan E M. Cell factories for insulin production. Microb Cell Fact. 2014; 13:141. Epub 2014/10/02. doi: 10.1186/s12934-014-0141-0. PubMed PMID: 25270715; PMCID: PMC4203937.
2. Norris K, Thim L, Norris F, Hansen M T, Moody A J. Process for preparing glucagon or fragments or derivatives thereof in yeast. Google Patents; 1989.
3. Nielsen J. Production of biopharmaceutical proteins by yeast: advances through metabolic engineering. Bioengineered. 2013; 4(4):207-11.
4. Stephenne J. Recombinant versus plasma-derived hepatitis B vaccines: issues of safety, immunogenicity and cost-effectiveness. Vaccine. 1988; 6(4):299-303.
5. Stephenne J. Development and production aspects of a recombinant yeast-derived hepatitis B vaccine. Vaccine. 1990; 8:S69-S73.
6. Chen W-H, Du L, Chag S M, Ma C, Tricoche N, Tao X, Seid C A, Hudspeth E M, Lustigman S, Tseng C-T K. Yeast-expressed recombinant protein of the receptor-binding domain in SARS-CoV spike protein with deglycosylated forms as a SARS vaccine candidate. Human vaccines & immunotherapeutics. 2014; 10(3):648-58.
7. Chen W-H, Chag S M, Poongavanam M V, Biter A B, Ewere E A, Rezende W, Seid C A, Hudspeth E M, Pollet J, McAtee C P. Optimization of the production process and characterization of the yeast-expressed SARS-CoV recombinant receptor-binding domain (RBD219-N1), a SARS vaccine candidate. Journal of pharmaceutical sciences. 2017; 106(8): 1961-70.
8. Kumar R, Kumar P. Yeast-based vaccines: New perspective in vaccine development and application. FEMS yeast research. 2019; 19(2):foz007.
9. Muscarella P, Wilfong L S, Ross S B, Richards D A, Raynov J, Fisher W E, Flynn P J, Whiting S H, Rosemurgy A, Harrell F E. A randomized, placebo-controlled, double blind, multicenter phase II adjuvant trial of the efficacy, immunogenicity, and safety of GI-4000 plus gem versus gem alone in patients with resected pancreas cancer with activating RAS mutations/survival and immunology analysis of the R1 subgroup. American Society of Clinical Oncology; 2012.
10. Chaft J E, Litvak A, Arcila M E, Patel P, D'Angelo S P, Krug L M, Rusch V, Mattson A, Coeshott C, Park B. Phase II study of the GI-4000 KRAS vaccine after curative therapy in patients with stage I-III lung adenocarcinoma harboring a KRAS G12C, G12D, or G12V mutation. Clinical lung cancer. 2014; 15(6):405-10.
11. Duke R C, Franzusoff A, Haller A, King T H, Lu Y, Hodson V K. Yeast-based vaccine for inducing an immune response. Google Patents; 2007.
12. Wang X, Wang Z, Xu H, Xiang B, Dang R, Yang Z. Orally administrated whole yeast vaccine against porcine epidemic diarrhea virus induced high levels of IgA response in mice and piglets. Viral Immunology. 2016; 29(9):526-31.
13. Kim H, Lee J, Kang H, Lee Y, Park E J, Kim H J. Oral immunization with whole yeast producing viral capsid antigen provokes a stronger humoral immune response than purified viral capsid antigen. Letters in applied microbiology. 2014; 58(3):285-91.
14. Bal J, Luong N N, Park J, Song K-D, Jang Y-S, Kim D-H. Comparative immunogenicity of preparations of yeast-derived dengue oral vaccine candidate. Microbial cell factories. 2018; 17(1):1-14.
15. Bagherpour G, Ghasemi H, Zand B, Zarei N, Roohvand F, Ardakani E M, Azizi M, Khalaj V. Oral administration of recombinant Saccharomyces boulardii expressing ovalbumin-CPE fusion protein induces antibody response in mice. Frontiers in microbiology. 2018; 9:723.
16. Ro D-K, Paradise E M, Ouellet M, Fisher K J, Newman K L, Ndungu J M, Ho K A, Eachus R A, Ham T S, Kirby J. Production of the antimalarial drug precursor artemisinic acid in engineered yeast. Nature. 2006; 440(7086):940-3.
17. Kurian J V. A new polymer platform for the future—Sorona® from corn derived 1, 3-propanediol. Journal of Polymers and the Environment. 2005; 13(2):159-67.
18. Galanie S, Thodey K, Trenchard I J, Interrante M F, Smolke C D. Complete biosynthesis of opioids in yeast. Science. 2015; 349(6252):1095-100.
19. Awan A R, Blount B A, Bell D J, Shaw W M, Ho J C, McKiernan R M, Ellis T. Biosynthesis of the antibiotic nonribosomal peptide penicillin in baker's yeast. Nature communications. 2017; 8(1):1-8.
20. Luo X, Reiter M A, d'Espaux L, Wong J, Denby C M, Lechner A, Zhang Y, Grzybowski A T, Harth S, Lin W. Complete biosynthesis of cannabinoids and their unnatural analogues in yeast. Nature. 2019; 567(7746):123-6.
21. Bond C M, Tang Y. Engineering Saccharomyces cerevisiae for production of simvastatin. Metabolic engineering. 2019; 51:1-8.
22. Lin H, Abida W M, Sauer R T, Cornish V W. Dexamethasone-Methotrexate: an efficient chemical inducer of protein dimerization in vivo. Journal of the American Chemical Society. 2000; 122(17):4247-8.
23. Lin H, Tao H, Cornish V W. Directed evolution of a glycosynthase via chemical complementation. Journal of the American Chemical Society. 2004; 126(46):15051-9.
24. Romanini D W, Peralta-Yahya P, Mondol V, Cornish V W. A heritable recombination system for synthetic Darwinian evolution in yeast. ACS synthetic biology. 2012; 1(12):602-9.
25. Ostrov N, Jimenez M, Billerbeck S, Brisbois J, Matragrano J, Ager A, Cornish VW. A modular yeast biosensor for low-cost point-of-care pathogen detection. Science advances. 2017; 3(6):e1603221.
26. Detmer A, Glenting J. Live bacterial vaccines—a review and identification of potential hazards. Microbial cell factories. 2006; 5(1):23.
27. Franzusoff A, Duke R C, King T H, Lu Y, Rodell T C. Yeasts encoding tumour antigens in cancer immunotherapy. Expert opinion on biological therapy. 2005; 5(4):565-75.
28. Munson S, Parker J, King T H, Lu Y, Kelley V, Guo Z, Borges V, Franzusoff A. Coupling innate and adaptive immunity with yeast-based cancer immunotherapy. Wiley Online Library; 2008. p. 131-49.
29. Kelesidis T, Pothoulakis C. Efficacy and safety of the probiotic Saccharomyces boulardii for the prevention and therapy of gastrointestinal disorders. Therapeutic advances in gastroenterology. 2012; 5(2): 111-25.
30. Venugopalan V, Shriner K A, Wong-Beringer A. Regulatory oversight and safety of probiotic use. Emerging infectious diseases. 2010; 16(11):1661.
31. Wrapp D, Wang N, Corbett K S, Goldsmith J A, Hsieh C-L, Abiona O, Graham B S, McLellan J S. Cryo-EM structure of the 2019-nCoV spike in the prefusion conformation. Science. 2020; 367(6483):1260-3.
32. Billerbeck S, Brisbois J, Agmon N, Jimenez M, Temple J, Shen M, Boeke J D, Cornish V W. A scalable peptide-GPCR language for engineering multicellular communication. Nature communications. 2018; 9(1): 1-11.
33. Sengupta D, Lin H, Goldberg S D, Mahal J J, Cornish V W. Correlation between catalytic efficiency and the transcription read-out in chemical complementation: a general assay for enzyme catalysis. Biochemistry. 2004; 43(12):3570-81.
34. Ng Y Z, Baldera-Aguayo P A, Cornish V W. Fluorescence Polarization Assay for Small Molecule Screening of FK506 Biosynthesized in 96-Well Microtiter Plates. Biochemistry. 2017; 56(40):5260-8.
35. Herbst E, Baldera-Aguayo P A, Lee H, Cornish V W. A Yeast Three Hybrid Assay for Metabolic Engineering of Tetracycline Derivatives. Biochemistry. 2018; 57(31):4726-34.

The above disclosed subject matter is to be considered illustrative, and not restrictive, and the appended claims are intended to cover all such modifications, enhancements, and other implementations which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

The contents of all figures and all references, patents and published patent applications and Accession numbers cited throughout this application are expressly incorporated herein by reference.

1. A fungal cell genetically engineered to produce an antigen comprising a protein or fragment thereof derived from a coronavirus in situ, wherein the antigen is secreted from the fungal cell.

2. The genetically-engineered fungal cell of claim 1, wherein the coronavirus is SARS-CoV-2, MERS-CoV or SARS-CoV.

3. The genetically-engineered fungal cell of claim 1 or 2, wherein the coronavirus is SARS-CoV-2.

4. The genetically-engineered fungal cell of any one of claims 1-3, wherein the coronavirus is a variant of SARS-CoV-2.

5. The genetically-engineered fungal cell of claim 4, wherein the variant of SARS-CoV-2 is selected from the group consisting of an alpha variant, a beta variant, a gamma variant, a delta variant, an epsilon variant and a combination thereof.

6. The genetically-engineered fungal cell of claim 4 or 5, wherein the variant of SARS-CoV-2 is selected from the group consisting of B.1.1.7, B.1.351, P.1, B.1.427, B.1.429, B.1.617.2 and a combination thereof.

7. The genetically-engineered fungal cell of any one of claims 1-6, wherein the antigen is secreted from the fungal cell by a secretory pathway of the fungal cell.

8. The genetically-engineered fungal cell of any one of claims 1-7, wherein the antigen is secreted from the fungal cell by the alpha-mating factor secretion pathway of the fungal cell.

9. The genetically-engineered fungal cell of any one of claim 1-8, wherein the antigen comprises an S protein or fragment thereof derived from the coronavirus.

10. The genetically-engineered fungal cell of claim 9, wherein the S protein or fragment thereof is the receptor-binding domain (RBD) of the S protein.

11. The genetically-engineered fungal cell of claim 10, wherein the RBD comprises one or more amino acid substitutions as shown in Table 3.

12. The genetically-engineered fungal cell of claim 10 or 11, wherein the RBD comprises one or more amino acid substitutions to modify the glycosylation and disulfide bonds of the RBD.

13. The genetically-engineered fungal cell of any one of claims 1-12, wherein the antigen comprises an amino acid sequence that is about 80%, about 85%, about 90% or about 95% homologous to an amino acid sequence set forth in SEQ ID NOs: 12-22.

14. The genetically-engineered fungal cell of any one of claims 1-13, wherein the antigen comprises an amino acid sequence set forth in SEQ ID NOs: 12-22 or conservative substitutions thereof.

15. The genetically-engineered fungal cell of any one of claims 1-13, wherein the antigen is encoded by a nucleotide sequence that is about 70%, about 75%, about 80%, about 85%, about 90% or about 95% homologous to a nucleotide sequence set forth in SEQ ID NOs: 1-11.

16. The genetically-engineered fungal cell of any one of claims 1-15, wherein the antigen is encoded by a nucleotide sequence set forth in SEQ ID NOs: 1-11.

17. The genetically-engineered fungal cell of any one of claim 1-16, wherein the genetically-engineered fungal cell further expresses and/or secretes a cell targeting molecule.

18. The genetically-engineered fungal cell of any one of claim 1-17, wherein the antigen further comprises a cell targeting molecule.

19. The genetically-engineered fungal cell of claim 17 or 18, wherein the cell targeting molecule is a molecule for targeting the genetically-engineered fungal cell and/or antigen to an immune cell.

20. The genetically-engineered fungal cell of claim 19, wherein the immune cell is located in a mucosal membrane and/or located in the Peyer's patches in the small intestine.

21. The genetically-engineered fungal cell of any one of claims 17-20, wherein the cell targeting molecule is selected from the group consisting of a C-terminal Clostridium perfringens Enterotoxin (C-CPE), a Cholera Toxin Subunit B (CTB), a heat-labile enterotoxin B subunit (LTB), a C-terminal Clostridium difficile toxin A (TxA(C314)), CRM197 (a diphtheria toxin mutant), Fragment C of tetanus toxoid, a cholesteryl group-bearing pullulan (CHP), PT-9K/129G (a detoxified derivative of pertussis toxin) or mutants thereof and a combination thereof.

22. The genetically-engineered fungal cell of any one of claims 17-21, wherein the cell targeting molecule comprises a CTB.

23. The genetically-engineered fungal cell of any one of claims 17-22, wherein the cell targeting molecule is coupled to the protein or fragment thereof derived from a coronavirus by a linker, e.g., an N-terminal linker, a C-terminal linker or a combination thereof.

24. The genetically-engineered fungal cell of claim 23, wherein the linker comprises Gly (G) and Ser (S) amino acids.

25. The genetically-engineered fungal cell of any one of claims 1-24, wherein the genetically-engineered fungal cell further expresses and/or secretes an adjuvant.

26. The genetically-engineered fungal cell of any one of claims 1-25, wherein the fungal cell is one or more species from a genus selected from the group consisting of Cladosporium, Aureobasidium, Aspergillus, Saccharomyces, Malassezia, Epicoccum, Candida, Penicillium, Wallemia, Pichia, Phoma, Cryptococcus, Fusarium, Clavispora, Cyberlindnera, Kluyveromyces, Hansenula, Yarrowia, Neurospora, Schizosaccharomyces, Blastobotrys, Zygosaccharomyces, Debaryomyces, Torulaspora, Hanseniaspora, Rhodotorula, Wickerhamomyces and Williopsis and a combination thereof.

27. The genetically-engineered fungal cell of claim 26, wherein the fungal cell is selected from the group consisting of Saccharomyces cerevisiae, Saccharomyces boulardii, Saccharomyces mikatae, Saccharomyces kudriavzevii, Saccharomyces uvarum, Kluyveromyces lactis, Kluyveromyces marxianus, Kluyveromyces dobzhansky, Hansenula polymorpha, Yarrowia lipolytica, Neurospora crassa, Schizosaccharomyces pombe, Blastobotrys (Arxula) adeninivorans, Candida boldmu, Candida boidinii, Pichia pastoris, Pichia methanolica, Pichia stipites, Zygosaccharomyces rouxii, Zygosaccharomyces bailii and Schwanniomyces (Debaryomyces) occidentalis.

28. The genetically-engineered fungal cell of claim 27, wherein the fungal cell is Saccharomyces cerevisiae, Saccharomyces boulardii and/or Kluyveromyces lactis.

29. A method for preventing and/or reducing the severity of a coronavirus infection in a subject comprising administering to the subject the genetically-engineered cell of any one of claims 1-28.

30. The method of claim 29, wherein the coronavirus is SARS-CoV-2, MERS-CoV or SARS-CoV.

31. The method of claim 29 or 30, wherein the coronavirus is SARS-CoV-2.

32. The method of any one of claims 29-31, wherein the coronavirus is a variant of SARS-CoV-2.

33. The method of claim 32, wherein the variant of SARS-CoV-2 is selected from the group consisting of an alpha variant, a beta variant, a gamma variant, a delta variant, an epsilon variant and a combination thereof.

34. The method of claim 32 or 33, wherein the variant of SARS-CoV-2 is selected from the group consisting of B.1.1.7, B.1.351, P.1, B.1.427, B.1.429, B.1.617.2 and a combination thereof.

35. The method of any one of claims 29-34, wherein the genetically-engineered fungal cell is formulated for parenteral administration, intraocular administration, intraaural administration, intranasal administration, oral administration, enteral administration, rectal administration, vaginal administration or topical administration.

36. The method of claim 35, wherein the genetically-engineered fungal cell is formulated for enteral administration.

37. The method of claim 35, wherein the genetically-engineered fungal cell is formulated for intranasal administration.

38. The method of any one of claims 29-37, wherein the genetically-engineered fungal cell is administered once every two weeks.

39. The method of any one of claims 29-38, further comprising the administration of an adjuvant.

40. A pharmaceutical composition comprising one or more genetically-engineered fungal cells of any one of claims 1-28 and a pharmaceutically acceptable carrier.

41. The pharmaceutical composition of claim 40, wherein the pharmaceutical composition is formulated for parenteral administration, intraocular administration, intraaural administration, intranasal administration, oral administration, enteral administration, rectal administration, vaginal administration or topical administration.

42. The pharmaceutical composition of claim 41, wherein the pharmaceutical composition is formulated for enteral administration.

43. The pharmaceutical composition of claim 41, wherein the pharmaceutical composition is formulated for intranasal administration.

44. The pharmaceutical composition of any one of claims 40-43, wherein the pharmaceutical composition further comprises a cell targeting molecule.

45. The pharmaceutical composition of any one of claims 40-44, wherein the pharmaceutical composition further comprises an adjuvant.

46. A kit comprising the genetically-engineered cell of any one of claims 1-28.

47. A kit comprising the pharmaceutical composition of any one of claims 40-45.

48. The genetically-engineered cell of any one of claims 1-28 or the pharmaceutical compositions of any one of claims 40-45 for use in preventing and/or reducing the severity of a coronavirus infection.

49. The genetically-engineered cell for use of claim 48, wherein the coronavirus is SARS-CoV-2, MERS-CoV or SARS-CoV.

50. The genetically-engineered cell for use of claim 49, wherein the coronavirus is SARS-CoV-2.

51. The genetically-engineered cell for use of claim 50, wherein the coronavirus is a variant of SARS-CoV-2.

52. The genetically-engineered cell for use of claim 51, wherein the variant of SARS-CoV-2 is selected from the group consisting of an alpha variant, a beta variant, a gamma variant, a delta variant, an epsilon variant and a combination thereof.

53. The genetically-engineered cell for use of claim 51 or 52, wherein the variant of SARS-CoV-2 is selected from the group consisting of B.1.1.7, B.1.351, P.1, B.1.427, B.1.429, B.1.617.2 and a combination thereof.


SEQUENCE LISTING (43 sequences)

SEQ ID NO: 1 - DNA, 3822 bp, Severe acute respiratory syndrome coronavirus 2
  source 1..3822: mol_type = genomic DNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 2 - DNA, 876 bp, Severe acute respiratory syndrome coronavirus 2
  source 1..876: mol_type = genomic DNA
cagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatcc

SEQ ID NO: 3 - DNA, 819 bp, Severe acute respiratory syndrome coronavirus 2
  source 1..819: mol_type = genomic DNA
agagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttct

SEQ ID NO: 4 - DNA, 669 bp, Severe acute respiratory syndrome coronavirus 2
  source 1..669: mol_type = genomic DNA
agagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttc

SEQ ID NO: 5 - DNA, 669 bp, Severe acute respiratory syndrome coronavirus 2
  source 1..669: mol_type = genomic DNA
agagtccaaccaacagaatctattgttagatttcctgctattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaagctgtcaatttc

SEQ ID NO: 6 - DNA, 594 bp, Severe acute respiratory syndrome coronavirus 2
  source 1..594: mol_type = genomic DNA
tttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtgga

SEQ ID NO: 7 - DNA, 594 bp, Severe acute respiratory syndrome coronavirus 2
  source 1..594: mol_type = genomic DNA
tttcctgctattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtgga

SEQ ID NO: 8 - DNA, 594 bp, Severe acute respiratory syndrome coronavirus 2
  source 1..594: mol_type = genomic DNA
tttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccacttatggtgttggttaccaaccatacagagtagtagtactttcttttgaacttctacatgcaccagcaactgtttgtgga

SEQ ID NO: 9 - DNA, 594 bp, Severe acute respiratory syndrome coronavirus 2
  source 1..594: mol_type = genomic DNA
tttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaatattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttaaaggttttaattgttactttcctttacaatcatatggtttccaacccacttatggtgttggttaccaaccatacagagtagtagtactttcttttgaacttctacatgcaccagcaactgtttgtgga

SEQ ID NO: 10 - DNA, 594 bp, Severe acute respiratory syndrome coronavirus 2
  source 1..594: mol_type = genomic DNA
tttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaacgattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttaaaggttttaattgttactttcctttacaatcatatggtttccaacccacttatggtgttggttaccaaccatacagagtagtagtactttcttttgaacttctacatgcaccagcaactgtttgtgga

SEQ ID NO: 11 - DNA, 594 bp, Severe acute respiratory syndrome coronavirus 2; identical to SEQ ID NO: 10
  source 1..594: mol_type = genomic DNA

SEQ ID NO: 12 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 13 - protein, 292 aa, Severe acute respiratory syndrome coronavirus 2
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKS

SEQ ID NO: 14 - protein, 273 aa, Severe acute respiratory syndrome coronavirus 2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCS

SEQ ID NO: 15 - protein, 223 aa, Severe acute respiratory syndrome coronavirus 2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 16 - protein, 223 aa, Severe acute respiratory syndrome coronavirus 2
RVQPTESIVRFPAITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKAVNF

SEQ ID NO: 17 - protein, 198 aa, Severe acute respiratory syndrome coronavirus 2
FPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCG

SEQ ID NO: 18 - protein, 198 aa, Severe acute respiratory syndrome coronavirus 2
FPAITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCG

SEQ ID NO: 19 - protein, 198 aa, Severe acute respiratory syndrome coronavirus 2
FPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCG

SEQ ID NO: 20 - protein, 198 aa, Severe acute respiratory syndrome coronavirus 2
FPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCG

SEQ ID NO: 21 - protein, 198 aa, Severe acute respiratory syndrome coronavirus 2
FPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCG

SEQ ID NO: 22 - protein, 198 aa, Severe acute respiratory syndrome coronavirus 2; identical to SEQ ID NO: 21

SEQ ID NO: 23 - DNA, 378 bp, Clostridium perfringens
  source 1..378: mol_type = genomic DNA
gatatagaaaaagaaatccttgatttagctgctgctacagaaagattaaatttaactgatgcattaaactcaaatccagctggtaatttatatgattggcgttcttctaactcatacccttggactcaaaagcttaatttacacttaacaattacagctactggacaaaaatatagaatcttagctagcaaaattgttgattttaatatttattcaaataattttaataatctagtgaaattagaacagtccttaggtgatggagtaaaagatcattatgttgatataagcttagatgctggacaatatgttcttgtaatgaaagctaattcatcatatagtggaaattacccttattcaatattatttcaaaaattt

SEQ ID NO: 24 - protein, 126 aa, Clostridium perfringens
DIEKEILDLAAATERLNLTDALNSNPAGNLYDWRSSNSYPWTQKLNLHLTITATGQKYRILASKIVDFNIYSNNFNNLVKLEQSLGDGVKDHYVDISLDAGQYVLVMKANSSYSGNYPYSILFQKF

SEQ ID NO: 25 - DNA, 375 bp, Vibrio cholerae
  source 1..375: mol_type = genomic DNA
atgattaaattaaaatttggtgttttttttacagttttactatcttcagcatatgcacatggaacacctcaaaatattactgatttgtgtgcagaataccacaacacacaaatacatacgctaaatgataagatattttcgtatacagaatctctagctggaaaaagagagatggctatcattacttttaagaatggtgcaacttttcaagtagaagtaccaggtagtcaacatatagattcacaaaaaaaagcgattgaaaggatgaaggataccctgaggattgcatatcttactgaagctaaagtcgaaaagttatgtgtatggaataataaaacgcctcatgcgattgccgcaattagtatggcaaattaa

SEQ ID NO: 26 - protein, 124 aa, Vibrio cholerae
MIKLKFGVFFTVLLSSAYAHGTPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMAN

SEQ ID NO: 27 - DNA, 372 bp, Escherichia coli
  source 1..372: mol_type = genomic DNA
atgaataaagtaaaatgttatgttttatttacggcgttactatcctctctatatgcacacggagctccccagactattacagaactatgttcggaatatcgcaacacacaaatatatacgataaatgacaagatactatcatatacggaatcgatggcaggcaaaagagaaatggttatcattacatttaagagcggcgaaacatttcaggtcgaagtcccgggcagtcaacatatagactcccagaaaaaagccattgaaaggatgaaggacacattaagaatcacatatctgaccgagaccaaaattgataaattatgtgtatggaataataaaacccccaattcaattgcggcaatcagtatgaaaaac

SEQ ID NO: 28 - protein, 124 aa, Escherichia coli
MNKVKCYVLFTALLSSLYAHGAPQTITELCSEYRNTQIYTINDKILSYTESMAGKREMVIITFKSGETFQVEVPGSQHIDSQKKAIERMKDTLRITYLTETKIDKLCVWNNKTPNSIAAISMKN

SEQ ID NO: 29 - protein, 15 aa, synthetic construct
  REGION 1..15: note = Description of Artificial Sequence: Syntheticpeptide
GGGGSGGGGSGGGGS

SEQ ID NO: 30 - protein, 5 aa, synthetic construct
  REGION 1..5: note = Description of Artificial Sequence: Syntheticpeptide
PAPAP

SEQ ID NO: 31 - protein, 12 aa, synthetic construct
  REGION 1..12: note = Description of Artificial Sequence: Syntheticpeptide
AEAAAKEAAAKA

SEQ ID NO: 32 - protein, 24 aa, synthetic construct
  REGION 1..24: note = Description of Artificial Sequence: Syntheticpeptide
AEAAAKEAAAKEAAAKEAAAKAAA

SEQ ID NO: 33 - protein, 6 aa, synthetic construct
  REGION 1..6: note = Description of Artificial Sequence: Synthetic6xHis tag
HHHHHH

SEQ ID NO: 34 - protein, 10 aa, synthetic construct
  REGION 1..10: note = Description of Artificial Sequence: Synthetic10xHis tag
HHHHHHHHHH

SEQ ID NO: 35 - DNA, 18 bp, synthetic construct
  misc_feature 1..18: note = Description of Artificial Sequence: Syntheticoligonucleotide
  source 1..18: mol_type = other DNA
catcatcaccaccaccat

SEQ ID NO: 36 - DNA, 1419 bp, Clostridium tetani
  source 1..1419: mol_type = genomic DNA
atgggcagcagccatcatcatcatcatcacagcagcggcctggtgccgcgcggcagccatatgaaaaaccttgattgttgggtcgacaacgaagaagacatcgatgttatcctgaaaaagtctaccattctgaacttggacatcaacaacgatattatctccgacatctctggtttcaactcctctgttatcacatatccagatgctcaattggtgccgggcatcaacggcaaagctatccacctggttaacaacgaatcttctgaagttatcgtgcacaaggccatggacatcgaatacaacgacatgttcaacaacttcaccgttagcttctggctgcgcgttccgaaagtttctgcttcccacctggaacagtacggcactaacgagtactccatcatcagctctatgaagaaacactccctgtccatcggctctggttggtctgtttccctgaagggtaacaacctgatctggactctgaaagactccgcgggcgaagttcgtcagatcactttccgcgacctgccggacaagttcaacgcgtacctggctaacaaatgggttttcatcactatcactaacgatcgtctgtcttctgctaacctgtacatcaacggcgttctgatgggctccgctgaaatcactggtctgggcgctatccgtgaggacaacaacatcactcttaagctggaccgttgcaacaacaacaaccagtacgtatccatcgacaagttccgtatcttctgcaaagcactgaacccgaaagagatcgaaaaactgtataccagctacctgtctatcaccttcctgcgtgacttctggggtaacccgctgcgttacgacaccgaatattacctgatcccggtagcttctagctctaaagacgttcagctgaaaaacatcactgactacatgtacctgaccaacgcgccgtcctacactaacggtaaactgaacatctactaccgacgtctgtacaacggcctgaaattcatcatcaaacgctacactccgaacaacgaaatcgattctttcgttaaatctggtgacttcatcaaactgtacgtttcttacaacaacaacgaacacatcgttggttacccgaaagacggtaacgctttcaacaacctggacagaattctgcgtgttggttacaacgctccgggtatcccgctgtacaaaaaaatggaagctgttaaactgcgtgacctgaaaacctactctgttcagctgaaactgtacgacgacaaaaacgcttctctgggtctggttggtacccacaacggtcagatcggtaacgacccgaaccgtgacatcctgatcgcttctaactggtacttcaaccacctgaaagacaaaatcctgggttgcgactggtacttcgttccgaccgatgaaggttggaccaacgactaa

SEQ ID NO: 37 - protein, 472 aa, Clostridium tetani
MGSSHHHHHHSSGLVPRGSHMKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 38 - DNA, 810 bp, synthetic construct
  misc_feature 1..810: note = Description of Artificial Sequence: Syntheticpolynucleotide
  source 1..810: mol_type = other DNA
atgcgttgcactcgggcaattcgccaaaccgcaagaacaggctggctgacgtggctggcgattcttgccgtcacggcgcccgtgacttcgccggcatgggccgacgatcctcccgccaccgtatacaaatatgactcccgcccgccggaggacgttttccagaacggattcacggcgtggggaaacaacgacaatgtgctcgaccatctgaccggacgttcctgccaggtcggcagcagcaacagcgctttcgtctccaccagcagcagccggcgctataccgaggtctatctcgaacatcgcatgcaggaagcggtcgaggccgaacgcgccggcaggggcaccggccacttcatcggctacatctacgaagtccgcgccgacaacaatttctacggcgccgccagctcgtacttcgaatacgtcgacacttatggcgacaatgccggccgtatcctcgccggcgcgctggccacctaccagagcggctatctggcacaccggcgcattccgcccgaaaacatccgcagggtaacgcgggtctatcacaacggcatcaccggcgagaccacgaccacggagtattccaacgctcgctacgtcagccagcagactcgcgccaatcccaacccctacacatcgcgaaggtccgtagcgtcgatcgtcggcacattggtgcgcatggcgccggtgataggcgcttgcatggcgcggcaggccgaaagctccgaggccatggcagcctggtccgaacgcgccggcgaggcgatggttctcgtgtactacgaaagcatcgcgtattcgttctag

SEQ ID NO: 39 - protein, 269 aa, synthetic construct
  REGION 1..269: note = Description of Artificial Sequence: Syntheticpolypeptide
MRCTRAIRQTARTGWLTWLAILAVTAPVTSPAWADDPPATVYKYDSRPPEDVFQNGFTAWGNNDNVLDHLTGRSCQVGSSNSAFVSTSSSRRYTEVYLEHRMQEAVEAERAGRGTGHFIGYIYEVRADNNFYGAASSYFEYVDTYGDNAGRILAGALATYQSGYLAHRRIPPENIRRVTRVYHNGITGETTTTEYSNARYVSQQTRANPNPYTSRRSVASIVGTLVRMAPVIGACMARQAESSEAMAAWSERAGEAMVLVYYESIAYSF

SEQ ID NO: 40 - DNA, 939 bp, Clostridium difficile
  source 1..939: mol_type = genomic DNA
attaatggtaaacatttttattttaatactgatggtattatgcagataggagtgtttaaaggacctaatggatttgaatactttgcacctgctaatacggatgctaacaacatagaaggtcaagctatactttaccaaaataaattcttaactttgaatggtaaaaaatattactttggtagtgactcaaaagcagttaccggactgcgaactattgatggtaaaaaatattactttaatactaacactgctgttgcagttactggatggcaaactattaatggtaaaaaatactactttaatactaacacttctatagcttcaactggttatacaattattagtggtaaacatttttattttaatactgatggtattatgcagataggagtgtttaaaggacctgatggatttgaatactttgcacctgctaatacagatgctaacaatatagaaggtcaagctatacgttatcaaaatagattcctatatttacatgacaatatatattattttggtaataattcaaaagcggctactggttgggtaactattgatggtaatagatattacttcgagcctaatacagctatgggtgcgaatggttataaaactattgataataaaaatttttactttagaaatggtttacctcagataggagtgtttaaagggtctaatggatttgaatactttgcacctgctaatacggatgctaacaatatagaaggtcaagctatacgttatcaaaatagattcctacatttacttggaaaaatatattactttggtaataattcaaaagcagttactggatggcaaactattaatggtaaagtatattactttatgcctgatactgctatggctgcagctggtggacttttcgagattgatggtgttatatatttctttggtgttgatggagtaaaagcccct

SEQ ID NO: 41 - protein, 313 aa, Clostridium difficile
INGKHFYFNTDGIMQIGVFKGPNGFEYFAPANTDANNIEGQAILYQNKFLTLNGKKYYFGSDSKAVTGLRTIDGKKYYFNTNTAVAVTGWQTINGKKYYFNTNTSIASTGYTIISGKHFYFNTDGIMQIGVFKGPDGFEYFAPANTDANNIEGQAIRYQNRFLYLHDNIYYFGNNSKAATGWVTIDGNRYYFEPNTAMGANGYKTIDNKNFYFRNGLPQIGVFKGSNGFEYFAPANTDANNIEGQAIRYQNRFLHLLGKIYYFGNNSKAVTGWQTINGKVYYFMPDTAMAAAGGLFEIDGVIYFFGVDGVKAP

SEQ ID NO: 42 - DNA, 1611 bp, synthetic construct
  misc_feature 1..1611: note = Description of Artificial Sequence: Syntheticpolynucleotide
  source 1..1611: mol_type = other DNA
atgggcgcagacgatgttgtggactcaagtaaatcatttgtcatggaaaacttctcctcatatcacggcacgaaaccgggctacgttgatagcattcagaaaggtatccaaaaaccgaaatctggcacgcagggtaactacgatgacgattggaaagaattctacagcaccgacaacaaatatgatgcggccggttactcagtcgacaacgaaaatccgctgtcgggcaaagccggcggtgtggttaaagtgacgtatccgggcctgaccaaagtcctggccctgaaagtggataatgcagaaaccatcaaaaaagaactgggtctgagcctgacggaaccgctgatggaacaggttggcaccgaagaatttatcaaacgcttcggcgatggtgccagtcgtgtcgtgctgtccctgccgttcgcagaaggtagctctagtgtggaatatattaacaattgggaacaagcgaaagccctgtccgttgaactggaaatcaactttgaaacccgcggcaaacgtggtcaggatgcgatgtatgaatacatggcacaagcttgcgcgggtaatcgcgttcgtcgcagcgtcggctcctcactgtcttgtatcaacctggactgggatgttatccgtgataaaaccaaaacgaaaatcgaaagtctgaaagaacatggcccgatcaaaaacaaaatgagcgaatctccgaataaaacggtgtccgaagaaaaagctaaacagtatctggaagaattccaccaaaccgcactggaacatccggaactgtcagaactgaaaaccgtgacgggtaccaacccggtttttgccggcgcaaattacgcagcttgggctgtgaacgttgcgcaagtgattgactcggaaacggccgataatctggaaaaaaccacggcggccctgagtattctgccgggcatcggttccgttatgggtattgccgacggcgcagtccatcacaacaccgaagaaattgtggcccagtctatcgcactgtcgagcctgatggttgctcaagcgattccgctggttggcgaactggttgatatcggctttgcagcttacaacttcgtggaaagtattatcaacctgtttcaggttgtccacaactcatataatcgcccggcctactcgccgggtcacaaaacccaaccgttcctgcatgacggctacgcggttagctggaatacggtcgaagattctattatccgtaccggctttcagggtgaatctggccacgacattaaaatcacggctgaaaacaccccgctgccgattgcaggtgttctgctgccgacgatcccgggtaaactggatgttaacaaatcaaaaacccatatctcggtcaacggtcgcaaaattcgtatgcgctgccgtgcgatcgacggcgatgtgaccttctgtcgtccgaaaagcccggtctatgtgggcaacggtgtccatgctaatctgcacgtggcgtttcatcgctctagttccgaaaaaatccatagtaacgaaatctcatcggattccattggtgtgctgggctaccagaaaaccgtggaccataccaaagtgaatagcaaactgagcctgttcttcgaaatcaaatcgtaa

SEQ ID NO: 43 - protein, 536 aa, synthetic construct
  REGION 1..536: note = Description of Artificial Sequence: Syntheticpolypeptide
MGADDVVDSSKSFVMENFSSYHGTKPGYVDSIQKGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS